(19) United States
(12) Patent Application Publication
(10) Pub. No.: US 2009/0306094 A1
(43) Pub. Date: Dec. 10, 2009

(54) Methods Of Identifying And Treating Individuals Exhibiting Mutant Bcr/Abl Kinase Polypeptides

(75) Inventors: Francis Y. Lee, Yardley, PA (US); Andreas Hochhaus, Ladenburg (DE); Martin Müller, Mannheim (DE)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/225,125

(22) PCT Filed: Mar. 16, 2007
(86) PCT No.: PCT/US2007/064149

§ 371 Date: Jul. 29, 2009

Related U.S. Application Data
(60) Provisional application No. 60/783,685, filed on Mar. 17, 2006.
(60) Provisional application No. 60/857,628, filed on Nov. 8, 2006.
(60) Provisional application No. 60/858,025, filed on Nov. 9, 2006.

(51) Int. Cl. A61K 31/496; C12Q 1/68; A61P 35/00

(52) U.S. Cl. 514/252.19; 435/6

(57) ABSTRACT

The present invention relates to mutant BCR-ABL kinase proteins, and to diagnostic and therapeutic methods and compositions useful in the management of disorders, for example cancers, involving cells that express such mutant BCR-ABL kinase proteins.

This application claims benefit to provisional application U.S. Ser. No. 60/783,685 filed Mar. 17, 2006; to provisional application U.S. Ser. No. 60/857,628 filed Nov. 8, 2006; and to provisional application U.S. Ser. No. 60/858,025, filed Nov. 9, 2006; under 35 U.S.C. 119(e). The entire teachings of the referenced applications are incorporated herein by reference.

### FIELD OF THE INVENTION

The present invention relates to mutant BCR-ABL kinase proteins, and to diagnostic and therapeutic methods and compositions useful in the management of disorders, for example cancers, involving cells that express such mutant BCR-ABL kinase proteins.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise and are predicted to become the leading cause of death in the developed world.

Chronic Myelogenous Leukemia (CML) is a myeloproliferative disorder that is characterized by Philadelphia chromosome translocation. (see, e.g. C. L. Sawyers, N. En. J. Med. 340, 1330 (1999); and S. Faderl et al., N. Engl. J. Med. 341,164 (1999)). A reciprocal translocation between chromosomes 9 and 22 produces the oncogenic BCR-ABL fusion protein. The BCL-ABL protein constitutes tyrosine kinase activity and is known to produce CML-like disease in mice (see, e.g. J. B. Konopka et al., Proc. Natl. Acad. Sci. U.S. 82, 1810 (1985); G. Q. Daley et al., Science 247, 824 (1990); and N. Heisterkamp et al., Nature 344, 251 (1990)). In fact, 95% of CML is Philadelphia-positive (Ph+). A single mutation on BCR-ABL is sufficient for the incidence of CML disease (D. G. Savage, K. H. Antman, NEJM 346(9) (2002).

CML progresses through distinct clinical stages. The earliest stage, termed chronic phase, is characterized by expansion of terminally differentiated neutrophils. Over several years the disease progresses to an acute phase termed blast crisis, characterized by maturation arrest with excessive numbers of undifferentiated myeloid or lymphoid progenitor cells. The BCR-ABL oncogene is expressed at all stages, but blast crisis is characterized by multiple additional genetic and molecular changes.

Imatinib mesylate (also known as STI-571) is a potent BCR-ABL tyrosine kinase inhibitor and is now standard of care in CML patients. As used herein the term “imatinib” is used to refer to imatinib mesylate or STI-571. Although imatinib is a potent inhibitor of the kinase activity of wild type BCR-ABL, many mutant BCR-ABL isoforms are resistant to clinically achievable doses of imatinib. Clinical resistance is primarily mediated by mutations within the kinase domain of BCR-ABL and, to a lesser extent, by amplification of the BCR-ABL genomic locus (M. E. Gorre et al., Science 193, 876 (2001)). Imatinib can bind to the adenosine triphosphate (ATP)-binding site of ABL only when its activation loop is “closed” and thus the protein is in inactive conformation. This conformation-specific requirement contributes to imatinib's selectivity and the resistance shown in CML patients (N. P. Shah et al., Cancer Cell 2, 117 (2002); which is hereby incorporated herein by reference in its entirety and for all purposes). The structure and use of imatinib as an anticancer agent is described in B. J. Druker et al., N. Engl. J. Med. 344, 1031 (2001) and S. G. O'Brien et al., N. Engl. J. Med. 348, 994 (2003), both of which are incorporated herein by reference in their entirety and for all purposes.

N-(2-chloro-6-methylphenyl)-2-(6-(4-(2-hydroxyethyl)piperazin-1-yl)-2-methylpyrimidin-4-ylamino)thiazole-5-carboxamide is a synthetic small-molecule inhibitor of several SRC-family kinases, including BCR-ABL. Structural studies indicate that protein tyrosine kinase inhibitors, including N-(2-chloro-6-methylphenyl)-2-[[6-[4-(2-hydroxyethyl)-1-piperazinyl]-2-methyl-4-pyrimidinyl]amino]-5-thiazolecarboxamide, bind to the ATP-binding site in ABL, but without regard for the position of the active loop, which can be in the active or inactive conformation (B. Nagar et al., Cancer Res. 62, 4236 (2002)). The less stringent conformation requirement for binding to the ABL kinase domain is one reason N-(2-chloro-6-methylphenyl)-2-[[6-[4-(2-hydroxyethyl)-1-piperazinyl]-2-methyl-4-pyrimidinyl]amino]-5-thiazolecarboxamide retains activity against many imatinib-resistant mutants. N-(2-chloro-6-methylphenyl)-2-[[6-[4-(2-hydroxyethyl)-1-piperazinyl]-2-methyl-4-pyrimidinyl]amino]-5-thiazolecarboxamide was more potent than imatinib at inhibiting nonmutated BCR-ABL (BCR-ABL/WT) kinase activity in a cell-based assay. In addition, the kinase activity of 14 out of 15 different clinically relevant, imatinib-resistant BCR-ABL isoforms was successfully inhibited in the low nanomolar range (N. P. Shah et al., Science 305, 399 (2003)).

The structure and use of N-(2-chloro-6-methylphenyl)-2-[[6-[4-(2-hydroxyethyl)-1-piperazinyl]-2-methyl-4-pyrimidinyl]amino]-5-thiazolecarboxamide as an anticancer agent is described in Lombardo, L. J., et al., J. Med. Chem., 47:6658-6661 (2004) and is described in the following US patents and pending applications: U.S. Pat. No. 6,596,746, granted Jul. 22, 2003; U.S. Pat. No. 7,125,875, granted Oct. 24, 2006, all of which are incorporated by reference herein in their entirety.

In view of the resistance of certain BCR-ABL mutants to drug therapy with imatinib, there is a need for a further understanding and identification of BCR-ABL mutants that may be resistant to other kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide. The invention provided herein satisfies this and other needs.

### SUMMARY OF THE INVENTION

The present inventors have discovered that mutations to the BCR-ABL polypeptide can render the polypeptide at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide.

The present invention provides methods of identifying subjects that have mutant BCR-ABL polypeptides, and in particular, BCR-ABL polypeptides having a mutation at position 507. In particular, the present invention provides methods of identifying subjects that have a E507G mutation. In certain aspects, the present invention provides methods of identifying subjects that have a E507G mutation. The invention further provides methods of identifying subject that have, not only the E507G mutation, but any number of additional mutations that are associated with at least partial resistant to drug therapy, including therapy with imatinib and/or N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof.

The present invention also provides methods of treating such subjects by tailoring their treatment regimen depending on whether or not they harbor mutant BCR-ABL polypeptides, and in particular, BCR-ABL polypeptides having at least a E507G mutation.

The present invention also provides mutant BCR-ABL polypeptides having at least a E507G mutation and polynucleotides encoding such polypeptides. The present invention further provides mutant BCR-ABL polypeptides having not only the E507G mutations but any number of additional mutations that are associated with at least partial resistance to drug therapy, including therapy with imatinib and/or N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof.

Methods of identifying compounds that can be used to treat BCR-ABL related disorders are also provided herein.

Methods for determining the responsiveness of an individual to therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof are provided herein. These methods can comprise the step of screening a biological sample from the individual for the presence of at least one mutation in a BCR-ABL kinase sequence wherein the presence of the mutation is indicative of the individual being at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof. In certain preferred embodiments of the present invention, the mutation is a E507G mutation.

Methods for treating an individual suffering from a BCR-ABL-associated disorder can comprise the steps of determining whether a biological sample obtained from the individual comprises a BCR-ABL kinase having at least one mutation, wherein the presence of the mutation is indicative of the individual being at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, and administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, to the individual. The therapeutically effective amount will depend upon whether or not the individual has the mutation and whether or not the therapy with N-(2-chloro-6-methylphenyl)-2-[[6-[4-(2-hydroxyethyl)-1-piperazinyl]-2-methyl-4-pyrimidinyl]amino]-5-thiazolecarboxamide will be combined with a second therapy. Currently, the recommended dosage for N-(2-chloro-6-methylphenyl)-2-[[6-[4-(2-hydroxyethyl)-1-piperazinyl]-2-methyl-4-pyrimidinyl]amino]-5-thiazolecarboxamide is twice daily as a 70 mg tablet referred to as SPRYCEL™. In certain embodiments, if an individual is determined to have a BCR-ABL mutant that renders cells partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide treatment, the dosage of the drug can be increased. Alternatively, the drug can be administered in combination with a second therapy for treating the BCR-ABL associated disorder. The second therapy can be any therapy effective in treating the disorder, including, for example, therapy with another protein kinase inhibitor such as imatinib, AMN107, PD180970, GGP76030, AP23464, SKI 606, NS-187, and/or AZD0530; therapy with a tubulin stabilizing agent for example, pacitaxol, epothilone, taxane, and the like; therapy with an ATP non-competitive inhibitor such as ONO12380; therapy with an Aurora kinase inhibitor such as VX-680; therapy with a p38 MAP kinase inhibitor such as BIRB-796; therapy with a BCR-ABL T315I inhibitor, or therapy with a farnysyl transferase inhibitor. The dosage of N-(2-chloro-6-methylphenyl)-2-[[6-[4-(2-hydroxyethyl)-1-piperazinyl]-2-methyl-4-pyrimidinyl]amino]-5-thiazolecarboxamide treatment or a pharmaceutically acceptable salt, hydrate, or solvate thereof can remain the same, be reduced, or be increased when combined with a second therapy.

The present invention provides methods for screening a biological sample, for example, a biological sample comprising cells that do not respond, or that have stopped responding, or that have a diminished response, to protein tyrosine kinase inhibitors. For example, the present invention provides a method of screening cells from an individual suffering from cancer who is being treated with imatinib and/or N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, and whose cells do not respond or have stopped responding or have a diminished response to either of the drugs, for the presence of BCR-ABL mutations described herein. The present invention provides certain BCR-ABL mutations that, if present, provide the basis upon which to alter treatment of such an individual.

An individual that is partially resistant to a protein tyrosine kinase inhibitor is an individual who has cells that have a diminished response to the protein tyrosine kinase inhibitor.

The methods of treating a BCR-ABL associated disorder in an individual suffering from cancer, will ideally inhibit proliferation of cancerous cells and/or induce apoptosis of the cancerous cells.

The individual to be screened or treated by the methods herein can be one that has received administration of a first kinase inhibitor to which the cancer cells in said individual have become resistant or at least partially resistant. The kinase inhibitor can be imatinib, N-(2-chloro-6-methylphenyl)-2-[[6-[4-(2-hydroxyethyl)-1-piperazinyl]-2-methyl-4-pyrimidinyl]amino]-5-thiazolecarboxamide, another kinase inhibitor, or any combination thereof. Alternatively, the individual will have not yet had treatment with a protein kinase inhibitor.

Combinations treatments comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are described in U.S. Ser. No. 10/886,955, filed Jul. 8, 2004, U.S. Ser. No. 11/265,843, filed Nov. 3, 2005, and U.S. Ser. No. 11/418,338, filed May 4, 2006, each of which are incorporated herein by reference in their entirety and for all purposes.

The invention comprises methods of establishing a treatment regimen for an individual having a BCR-ABL related disorder. The treatment regimen can comprise the administration of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, at a higher dose or dosing frequency than recommended for an individual having non-mutated BCR-ABL or a BCR-ABL polypeptide lacking the E507G mutation. Alternatively, the treatment regiment can comprise combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide and any other agent that works to inhibit proliferation of cancerous cells or induce apoptosis of cancerous cells, including, for example, a tubulin stabilizing agent, a farnysyl transferase inhibitor, a BCR-ABL T315I inhibitor and/or another protein tyrosine kinase inhibitor. Preferred other agents include imatinib, AMN107, PD180970, CGP76030, AP23464, SKI 606, NS-187, or AZD0530. The treatment regimen can include administration of a higher dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, a reduced dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, or an unchanged dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent.

The present invention provides a kit for use in determining treatment strategy for an individual with a protein tyrosine kinase-associated disorder, comprising a means for detecting a mutant BCR-ABL kinase in a biological sample from said patient; and optionally instructions for use and interpretation of the kit results. The kit can also comprise, for example, a means for obtaining a biological sample from an individual. The treatment strategy can comprise, for example, the administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof. In certain embodiments, the mutant kinase will comprise a mutation at position 507. In certain embodiments, the mutation at position 507 will be a E507G mutation.

The E507G mutation of a BCR-ABL protein can be indicative of a greater likelihood of having partial resistance to therapy with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof. Additional mutations may be present as well, including for example any combination of the mutations described herein, i.e., E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, T315A, E316G, F317L, F317I, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, and F486S, including for example, M244V, G250E, Q252H, Q252R, Y253F, Y253H, E255K, E255V, T315I, T315A, F317L, F317I, M351T, E355G, F359V, H396R, F486S; M244V, E279K, F359C, F359I, L364I, L387M, F486S and any combination thereof, and L248R, Q252H, E255K, V299L, T315I, T315A, F317I, F317V, F317L, F317S and any combination thereof.

The present invention not only provides screening and diagnostic methods but also polynucleotides encoding a BCR-ABL mutant polypeptide having substantial identity or exact identity to SEQ ID NO:2 except for the presence of at least one of the E507G mutation, and fragments thereof. The polynucleotide can encode a BCR-ABL mutant polypeptide having the E507G mutation and any combination of the additional mutations described herein. Also provided are BCR-ABL mutant polypeptides having substantial identity or exact identity to SEQ ID NO:2 except for the presence of at least the E507G mutation, and fragments thereof. The polypeptides of the present invention can have the E507G mutation and any combination of the additional mutations described herein. Antibodies directed to the mutant BCR-ABL polypeptides and methods of using the antibodies to detect the polypeptides are also included herein.

Methods of determining whether a test compound modulates, i.e., inhibits, the tyrosine kinase activity of a mutant BCR-ABL polypeptide can comprise the steps of obtaining mammalian cells transfected with a construct encoding the mutant BCR-ABL polypeptide, contacting the cells with the test compound, and monitoring the cells for tyrosine kinase activity of the mutant BCR-ABL polypeptide wherein a modulation, i.e., inhibition, in tyrosine kinase activity in the presence of the test compound identifies the test compound as a modulator, i.e., inhibitor, of the mutant BCR-ABL polypeptides.

Imatinib is a small-molecule inhibitor of the BCR/ABL tyrosine kinase that produces clinical remissions in CML patients with minimal toxicity relative to older treatment modalities. imatinib is now frontline therapy for CML but resistance is increasingly encountered. According to one study, the estimated 2-year incidence of resistance to imatinib mesylate was 80% in blastic phase, 40% to 50% in accelerated phase, and 10% in chronic phase post-interferon-α failure (Kantarjian et al, Blood, 101(2):473-475 (2003). N-(2-chloro-6-methylphenyl)-2-[[6-[4-(2-hydroxyethyl)-1-piperazinyl]-2-methyl-4-pyrimidinyl]amino]-5-thiazolecarboxamide is an ATP-competitive, dual SRC/ABL inhibitor (Lombardo, L. J., et al., J. Med. Chem., 47:6658-6661 (2004)). Notably, N-(2-chloro-6-methylphenyl)-2-[[6-[4-(2-hydroxyethyl)-1-piperazinyl]-2-methyl-4-pyrimidinyl]amino]-5-thiazolecarboxamide has been shown to inhibit BCR-ABL imatinib-resistant mutations that are found in some CML patients with acquired clinical resistance to imatinib. On account of the demonstration that patients harboring different Src mutations, particularly BCR/ABL mutations, have varying degrees of resistance and/or sensitivity to imatinib and N-(2-chloro-6-methylphenyl)-2-[[6-[4-(2-hydroxyethyl)-1-piperazinyl]-2-methyl-4-pyrimidinyl]amino]-5-thiazolecarboxamide, respectively, the inventors of the present invention describe for the first time methods to identify patients who may most benefit from the combination of N-(2-chloro-6-methylphenyl)-2-[[6-[4-(2-hydroxyethyl)-1-piperazinyl]-2-methyl-4-pyrimidinyl]amino]-5-thiazolecarboxamide with other protein tyrosine kinase inhibitors, or other agents. The present invention also provides mutant BCR-ABL polynucleotide and polypeptide compositions.

Therefore, the present invention provides compositions, kits and methods for diagnosing and treating a host, preferably human, having or predisposed to a disease associated with abnormal activity of one or more protein tyrosine kinases. Specifically, the invention provides methods of identifying a mutant BCR/ABL kinase in a host having a disease associated with abnormal activity of said BCR/ABL mutant kinase, and tailoring treatment of said host based upon identification of said mutant BCR/ABL kinase.

The present invention provides kits for screening and diagnosing disorders associated with aberrant or uncontrolled cellular development and with the expression of a SRC kinase mutant as described herein.

The present invention provides a method of screening a biological sample, for example cells that do not respond, or that have stopped responding, or that have a diminished response, to protein tyrosine kinase inhibitors used to inhibit proliferation of said cells. For example, the present invention provides a method of screening cells from an individual suffering from cancer who is being treated with imatinib, and whose cells do not respond or have stopped responding or that have a diminished response to imatinib, for the presence of BCR-ABL mutations described herein. The present invention provides certain BCR-ABL mutations that, if present, provide the basis upon which to alter treatment of such individual to inhibit proliferation of said cells.

The present invention provides a method of screening cells that do not respond, or that have stopped responding or that have a diminished response, to kinase inhibitors used to induce apoptosis of said cells. For example, the present invention provides a method of screening cells from an individual suffering from cancer who is being treated with imatinib or who are naive to imatinib, and whose cells do not respond or have stopped responding or that have a diminished response to imatinib, for the presence of BCR-ABL kinase mutations described herein. The present invention provides certain BCR-ABL kinase mutations that, if present, provide the basis upon which to alter treatment of such individual to induce apoptosis of said cells.

Also provided is a method of treating a BCR-ABL associated disorder, particularly a mutant BCR-ABL-associated disorder, comprising obtaining a sample of cells from a patient suffering from said disorder, assaying the cells for the presence of a BCR-ABL mutation, such as one or more of those described herein, and treating said patient with a combination or treatment regimen to inhibit proliferation and/or induce apoptosis of said cells.

The invention encompasses a method of treating an individual suffering from cancer, wherein the method comprises assaying cells from said individual to determine the presence of at least one mutation in a BCR-ABL kinase protein in said cells, wherein said at least one mutation in a BCR-ABL kinase results in said BCR-ABL kinase being constitutively activated, and thereby administering to said individual a therapeutically effective amount of a member of the group consisting of: a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor, especially a BCR-ABL inhibitor such as imatinib as indicated herein, or AMN107; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

The invention further encompasses a method of treating an individual suffering from cancer, wherein the method comprises assaying cells from said individual to determine the presence of at least one mutation in a BCR-ABL kinase protein in said cells, wherein said at least one mutation is at one or more acid positions of SEQ ID NO:2, and, if said mutation in said BCR-ABL protein kinase is identified, administering to said individual a therapeutically effective amount of a member of the group consisting of: a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

The invention further comprises a method of treating an individual suffering from cancer (especially a BCR-ABL kinase associated cancer), wherein said individual is or has received administration of a first kinase inhibitor to which the cancer cells in said individual have become resistant or at least partially resistant, comprising assaying cells from said individual to determine the presence of at least one mutation in BCR-ABL kinase protein in said cells, wherein said at least one mutation in a BCR-ABL kinase results in said cancer cells being resistant to said first kinase inhibitor, and, if at least one mutation is present in said BCR-ABL kinase protein, administering a therapeutically effective amount of a member of the group consisting of: a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib alone; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and said first kinase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

The invention further comprises a method of treating an individual suffering from cancer (especially a BCR-ABL associated cancer), wherein said individual is or has received administration of BCR-ABL inhibitor such as imatinib to which the cancer cells in said individual have become resistant or at least partially resistant, comprising assaying cells from said individual to determine the presence of at least one mutation in a SRC kinase protein in said cells, wherein said at least one mutation in a SRC kinase results in said cancer cells being resistant or at least partially resistant to imatinib, and, if at least one mutation is present in said SRC kinase protein, administering a therapeutically effective amount of a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another BCR-ABL inhibitor such as imatinib alone or in combination with other agents including, but not limited to Taxol or other protein tyrosine kinase inhibitors. Combinations comprising N-(2-chloro-6-methylphenyl)-2-[[6-[4-(2-hydroxyethyl)-1-piperazinyl]-2-methyl-4-pyrimidinyl]amino]-5-thiazolecarboxamide that may be useful to practice the methods of the present invention are described in U.S. Ser. No. 10/886,955, filed Jul. 8, 2004, U.S. Ser. No. 60/632,122, filed Dec. 1, 2004, and U.S. Ser. No. 60/678,030, filed May 5, 2005, each of which are incorporated herein by reference.

The invention further comprises a method of treating an individual suffering from cancer, wherein said individual is or has received administration of imatinib to which the cancer cells in said individual have become resistant or at least partially resistant, comprising assaying cells from said individual to determine the presence of at least one mutation in a SRC kinase protein in said cells, wherein said at least one mutation in a SRC kinase results in said cancer cells being resistant to imatinib or at least partially resistant, and, if at least one mutation is present in said SRC kinase protein, administering a therapeutically effective amount of a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, including an increased or decreased dose, alone or in combination with other agents including, but not limited to Taxol or other protein tyrosine kinase inhibitors. Combinations comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib that may be useful to practice the methods of the present invention are described in U.S. Ser. No. 10/886,955, filed Jul. 8, 2004, U.S. Ser. No. 60/632,122, filed Dec. 1, 2004, and U.S. Ser. No. 60/678,030, filed May 5, 2005, each of which are incorporated herein by reference.

The invention further comprises a method of treating an individual suffering from cancer (especially a BCR-ABL associated cancer), wherein said individual is or has received administration of a first kinase inhibitor to which the cancer cells in said individual have become resistant or at least partially resistant, comprising assaying cells from said individual to determine the presence of at least one mutation in a BCR-ABL kinase protein in said cells, wherein said at least one mutation is at one or more amino acid positions of SEQ ID NO:2, and, if said mutation is present, administering a therapeutically effective amount of a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib.

The invention further comprises a method of treating an individual suffering from cancer (especially a SRC associated cancer), wherein said individual is or has received administration of imatinib to which the cancer cells in said individual have become resistant or at least partially resistant, comprising assaying cells from said individual to determine the presence of at least one mutation in a SRC kinase protein in said cells, wherein said at least one mutation is at one or more amino acid positions of SEQ ID NO:2, and, if said mutation is present, administering a therapeutically effective amount of a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib.

The invention further relates to a nucleic acid comprising, or alternatively consisting of, a polynucleotide encoding a polypeptide of SEQ ID NO:2 comprising, one or more amino acid substitutions.

The invention further relates to a polypeptide comprising, or alternatively consisting of, a polypeptide of SEQ ID NO:2 comprising, one or more amino acid substitutions.

The invention further relates to a nucleic acid comprising, or alternatively consisting of, a polynucleotide encoding a polypeptide of SEQ ID NO:2 comprising, one or more amino acid substitutions, wherein said one or more amino acid substitutions results in the encoded polypeptide being at least partially resistant to a protein tyrosine kinase inhibitor.

The invention further relates to a polypeptide comprising, or alternatively consisting of, a polypeptide of SEQ ID NO:2 comprising, one or more amino acid substitutions, wherein said one or more amino acid substitutions results in said polypeptide being at least partially resistant to a protein tyrosine kinase inhibitor.

The invention further relates to an isolated nucleic acid molecule of SEQ ID NO:1, wherein the nucleotide sequence encodes a polypeptide comprising one or more deletions from either the C-terminus or the N-terminus.

The invention further relates to an isolated polypeptide of SEQ ID NO:2, wherein said polypeptide comprises one or more deletions from either the C-terminus or the N-terminus.

The present inventors have discovered that mutations to the BCR-ABL polypeptide can render the polypeptide at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide.

### BRIEF DESCRIPTION OF THE DRAWINGS

FIGS. 1A-D show the polynucleotide sequence (SEQ ID NO:1) and deduced amino acid sequence (SEQ ID NO:2) of the wild-type BCR-ABL polypeptide. The standard one-letter abbreviation for amino acids is used to illustrate the deduced amino acid sequence. The polynucleotide sequence contains a sequence of 3393 nucleotides (SEQ ID NO:1; gi|177942; and gi|M14752.1), encoding a polypeptide of 1130 amino acids (SEQ ID NO:2; g|177943; and gi|M14752.1).

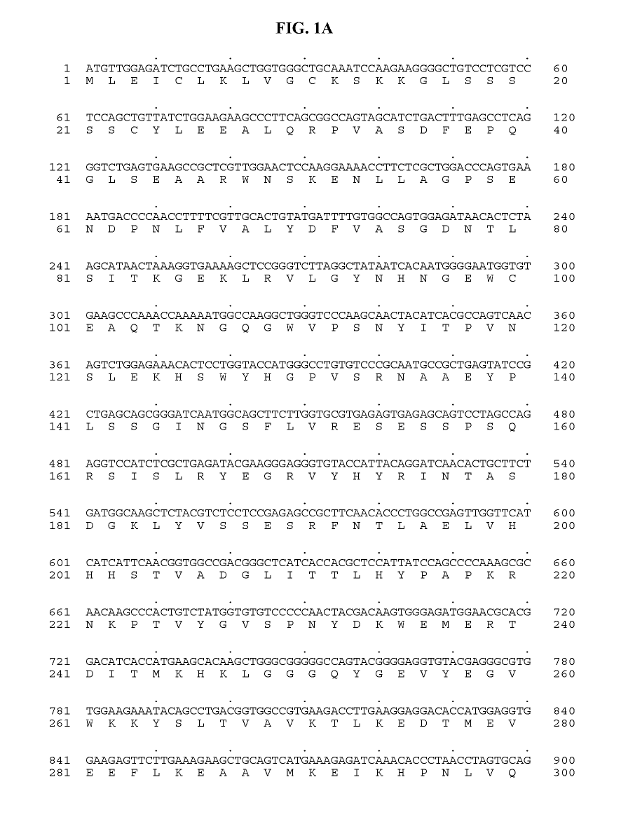

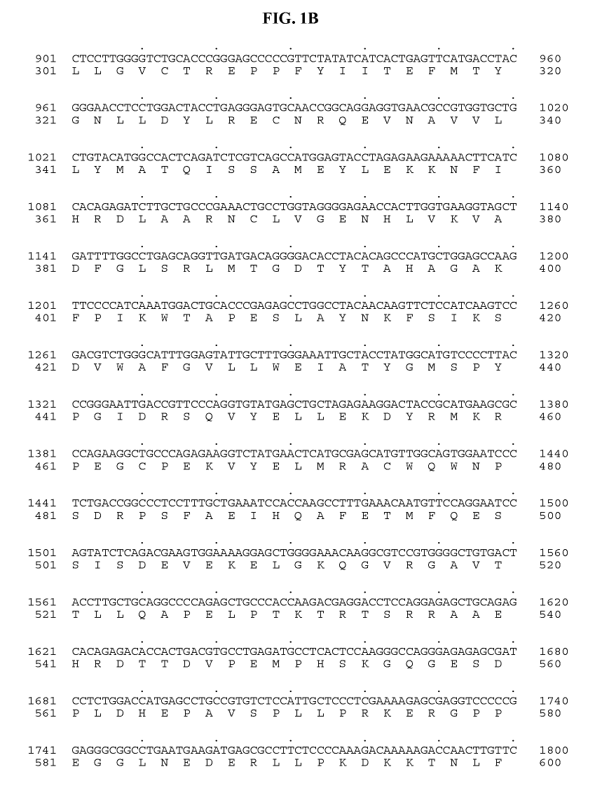

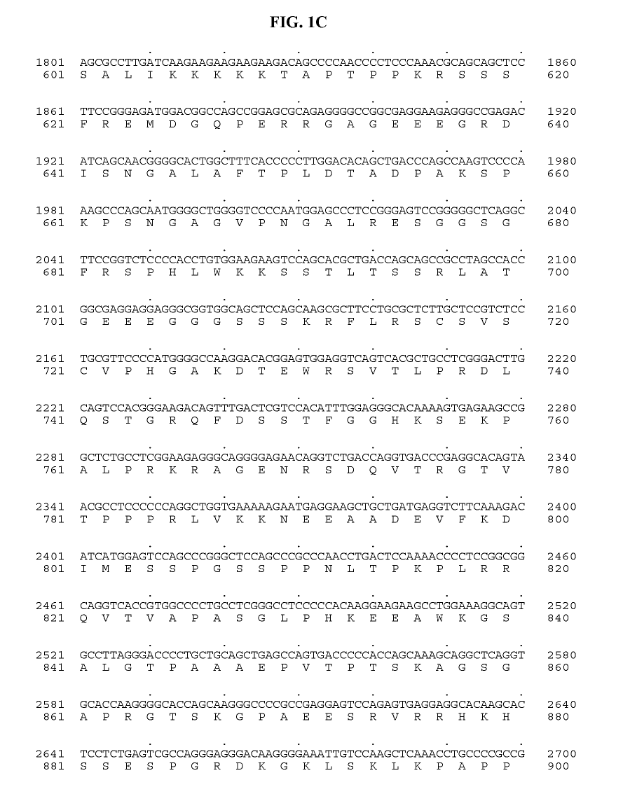

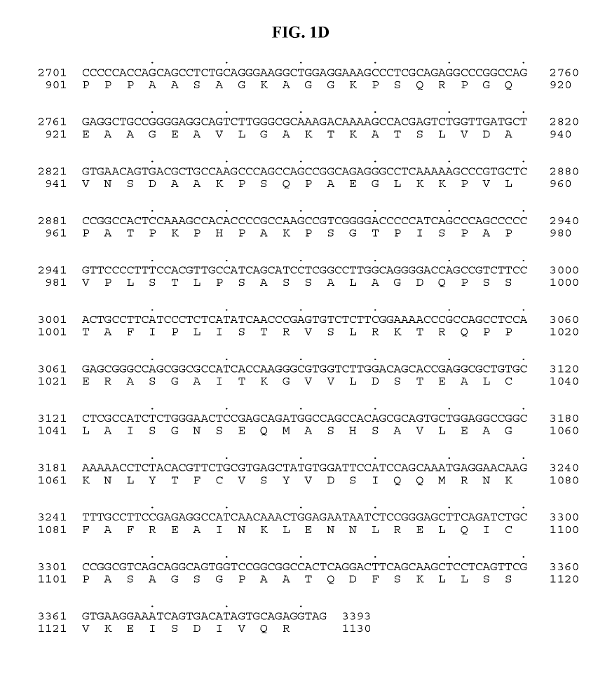

### DETAILED DESCRIPTION OF ILLUSTRATIVE EMBODIMENTS

### Introduction

The present invention is based, in part, on the discovery that certain individuals treated with N-(2-chloro-6-methylphenyl)-2-(6-(4-(2-hydroxyethyl)piperazin-1-yl)-2-methylpyrimidin-4-ylamino)thiazole-5-carboxamide and/or Imatinib develop mutations at select amino acid positions within the BCR-ABL kinase domain and that these mutations are associated with at least partial resistance to therapy with N-(2-chloro-6-methylphenyl)-2-(6-(4-(2-hydroxyethyl)piperazin-1-yl)-2-methylpyrimidin-4-ylamino)thiazole-5-carboxamide and/or Imatinib.

Recognition that these mutations exist in an individual having a BCR-ABL-associated disorder can, among other things, help in determining the responsiveness of individuals to treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, and/or Imatinib, and it can help tailor treatment regimens appropriately.

As is known in the art, N-(2-chloro-6-methylphenyl)-2-[[6-[4-(2-hydroxyethyl)-1-piperazinyl]-2-methyl-4-pyrimidinyl]amino]-5-thiazolecarboxamide refers to a compound having the following structure (I):

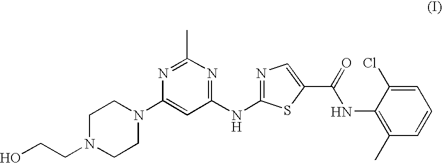

Compound (I) can also be referred to as N-(2-chloro-6-methylphenyl)-2-((6-(4-(2-hydroxyethyl)-1-piperazinyl)-2-methyl-4-pyrimidinyl)amino)-1,3-thiazole-5-carboxamide in accordance with FUPAC nomenclature. Use of the term “N-(2-chloro-6-methylphenyl)-2-[[6-[4-(2-hydroxyethyl)-1-piperazinyl]-2-methyl-4-pyrimidinyl]amino]-5-thiazolecarboxamide” encompasses (unless otherwise indicated) solvates (including hydrates) and polymorphic forms of the compound (I) or its salts (such as the monohydrate form of (I) described in U.S. Ser. No. 11/051,208, filed Feb. 4, 2005, incorporated herein by reference in its entirety and for all purposes). Pharmaceutical compositions of N-(2-chloro-6-methylphenyl)-2-[[6-[4-(2-hydroxyethyl)-1-piperazinyl]-2-methyl-4-pyrimidinyl]amino]-5-thiazolecarboxamide include all pharmaceutically acceptable compositions comprising N-(2-chloro-6-methylphenyl)-2-[[6-[4-(2-hydroxyethyl)-1-piperazinyl]-2-methyl-4-pyrimidinyl]amino]-5-thiazolecarboxamide and one or more diluents, vehicles and/or excipients, such as those compositions described in U.S. Ser. No. 11/402,502, filed Apr. 12, 2006, incorporated herein by reference in its entirety and for all purposes. One example of a pharmaceutical composition comprising N-(2-chloro-6-methylphenyl)-2-[[6-[4-(2-hydroxyethyl)-1-piperazinyl]-2-methyl-4-pyrimidinyl]amino]-5-thiazolecarboxamide is SPRYCEL™ (Bristol-Myers Squibb Company). SPRYCEL™ comprises N-(2-chloro-6-methylphenyl)-2-[[6-[4-(2-hydroxyethyl)-1-piperazinyl]-2-methyl-4-pyrimidinyl]amino]-5-thiazolecarboxamide as the active ingredient, also referred to as dasatinib, and as inactive ingredients or excipients, lactose monohydrate, microcrystalline cellulose, croscarmellose sodium, hydroxypropyl cellulose, and magnesium stearate in a tablet comprising hypromellose, titanium dioxide, and polyethylene glycol.

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

### Polypeptides, Polynucleotides, and Antibodies

The present invention provides isolated novel BCR-ABL nucleotides and their encoded proteins having mutations at certain amino acids that can render an individual at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt of hydrate thereof. At least one of the mutations is preferably a E507G mutation.

The single letter amino acid sequence of wild-type human BCR-ABL protein shown is known in the art and provided as SEQ ID NO:2. The nucleic acid sequence of BCR-ABL is encoded by nucleotides 1 to 3681 of SEQ ID NO:1.

For purposes of shorthand designation of the mutant variants described herein, it is noted that numbers refer to the amino acid residue position along the amino acid sequence of the BCR-ABL polypeptide as provided as SEQ ID NO:2. For example, E507 refers to the amino acid glutamic acid at position 507. Amino acid substitutions at a particular position are written as the wild type amino acid, position number, and amino acid substituted therein, in that order. For example, E507G refers to a substitution of glycine for glutamic acid at position 507. Amino acid identification uses the single-letter alphabet of amino acids, as shown in Table 1 below.

TABLE 1

| | | | | | |
|---|---|---|---|---|---|
| Asp | D | Aspartic acid | Ile | I | Isoleucine |
| Thr | T | Threonine | Leu | L | Leucine |
| Ser | S | Serine | Tyr | Y | Tyrosine |
| Glu | E | Glutamic acid | Phe | F | Phenylalanine |
| Pro | P | Proline | His | H | Histidine |
| Gly | G | Glycine | Lys | K | Lysine |
| Ala | A | Alanine | Arg | R | Arginine |
| Cys | C | Cysteine | Trp | W | Tryptophan |
| Val | V | Valine | Gln | Q | Glutamine |
| Met | M | Methionine | Asn | N | Asparagine |

Accordingly the present invention provides isolated novel BCR-ABL polypeptides comprising the amino acid sequence set forth in SEQ ID NO:2 or having substantial identity to the amino acid sequence set forth in SEQ ID NO:2 and having at least a E507G mutation, and fragments thereof. The present invention also provides polypeptides having at least a E507G mutation and one or more of the following mutations or any combination thereof: E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, T315A, E316G, F317L, F317I, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, or F486S, including for example, M244V, G250E, Q252H, Q252R, Y253F, Y253H, E255K, E255V, T315I, T315A, F317L, F317I, M351T, E355G, F359V, H396R, F486S; M244V, E279K, F359C, F359I, L364I, L387M, F486S and any combination thereof, and L248R, Q252H, E255K, V299L, T315I, T315A, F317V, F317I, F317L, F317S and any combination thereof.

The present invention also provides conservatively modified variants of SEQ ID NO:2 having at least a E507G mutation, and fragments thereof.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g. Creighton, Proteins (1984)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “polynucleotide” means a polymeric form of nucleotides of at least about 10 bases or base pairs in length, either ribonucleotides or deoxynucleotides or a modified form of either type of nucleotide, and is meant to include single and double stranded forms of DNA.

As used herein, a polynucleotide is said to be “isolated” when it is substantially separated from contaminant polynucleotides that correspond or are complementary to genes other than, the BCR-ABL gene or mutants thereof. As used herein, a polypeptide is said to be “isolated” when it is substantially separated from contaminant polypeptide that correspond to polypeptides other than the BCR-ABL peptide or mutant polypeptides or fragments thereof. A skilled artisan can readily employ polynucleotide or polypeptide isolation procedures well known in the art to obtain said isolated polynucleotides and/or polypeptides.

As used herein “substantial identity” to a specified sequence refers to 80% identity or greater, i.e., 81%, 82%, 83%, 84%, 85%, 86%, 87%, 88%, 89%, 90%, 91%, 91%, 93%, 94%, 95%, 96%, 97%, 98%, o99%, 99.5% or 99.9% identity to the specified sequence.

In the context of amino acid sequence comparisons, the term “identity” is used to identify and express the percentage of amino acid residues at the same relative positions that are the same. Also in this context, the term “homology” is used to identify and express the percentage of amino acid residues at the same relative positions that are either identical or are similar, using the conserved amino acid criteria of BLAST analysis, as is generally understood in the art. For example, identity and homology values can be generated by WU-BLAST-2 (Altschul et al., Methods in Enzymology, 266: 460-480 (1996): http://blast.wustl/edu/blast/README.html).

“Percent (%) amino acid sequence identity” with respect to the sequences identified herein is defined as the percentage of amino acid residues in a candidate sequence that are identical with the amino acid residues in the BCR-ABL sequence, after aligning the sequences and introducing gaps, if necessary, to achieve the maximum percent sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent ammo acid identity values can also be obtained using the sequence comparison computer program, ALIGN-2, the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

The polynucleotides of the invention are useful for a variety of purposes, including, for example, their use in the detection of the gene(s), mRNA(s), or fragments thereof, as reagents for the diagnosis and/or prognosis of BCR-ABL associated disorders, including cancers; as coding sequences capable of directing the expression of their encoded polypeptides; and as tools for modulating or inhibiting the function of the encoded protein.

Further specific embodiments of this aspect of the invention include primers and primer pairs, which allow the specific amplification of the polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers can be used to detect the presence of a polynucleotide of the present invention in a sample and as a means for detecting a cell expressing a protein of the present invention.

As used herein, the terms “hybridize”, “hybridizing”, “hybridizes” and the like, used in the context of polynucleotides, are meant to refer to conventional hybridization conditions, preferably such as hybridization in 50% formamide/6×SSC/0.1% SDS/100 μg/ml ssDNA, in which temperatures for hybridization are above 37° C. and temperatures for washing in 0.1×SSC/0.1% SDS are above 55° C., and most preferably to stringent hybridization conditions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, are known to those of skill in the art and as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

“Moderately stringent conditions” can be identified as described by Sambrook et al., 1989, Molecular Cloning: A Laboratory Manual, New York: Cold Spring Harbor Press, and include the use of washing solution and hybridization conditions (e.g., temperature, ionic strength and % SDS) less stringent than those described above. A non-limiting example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The invention also provides recombinant DNA or RNA molecules comprising a polynucleotide of the present invention, including, for example, phages, plasmids, phagemids, cosmids, YACs, BACs, as well as various viral and non-viral vectors well known in the art, and cells transformed or transfected with such recombinant DNA or RNA molecules. As used herein, a recombinant DNA or RNA molecule is a DNA or RNA molecule that has been subjected to molecular manipulation in vitro. Methods for generating such molecules are well known (see, for example, Sambrook et al, 1989, supra).

The invention further provides a host-vector system comprising a recombinant DNA molecule containing a polynucleotide of the present invention within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 cell). Examples of suitable mammalian cells include various cancer cell lines, other transfectable or transducible cell lines, including those mammalian cells routinely used for the expression of recombinant proteins (e.g., COS, CHO, 293, 293T cells and the like). More particularly, a polynucleotide encoding a mutant BCR-ABL of the present invention can be used to generate proteins or fragments thereof using any number of host vector systems routinely used and widely known in the art. Cell lines comprising the BCR-ABL polypeptides and BCR-ABL polynucleotides of the present invention are provided herein.

Proteins encoded by the genes of the present invention, or by fragments thereof, have a variety of uses, including, for example, generating antibodies and in methods for identifying ligands and other agents (e.g. small molecules such as 2-phenylpyrimidines) and cellular constituents that bind to a gene product. Antibodies raised against a BCR-ABL mutant protein or fragment thereof are useful in diagnostic and prognostic assays, imaging methodologies (including, particularly, cancer imaging), and therapeutic methods in the management of human cancers characterized by expression of a protein of the present invention, including, for example, cancer of the lymphoid lineages. Various immunological assays useful for the detection of proteins of the present invention are contemplated, including, for example, various types of radioimmunoassays, enzyme-linked immunosorbent assays (ELISA), enzyme-linked immunofluorescent assays (ELIFA), immunocytochemical methods, and the like. Such antibodies can be labeled and used as immunological imaging reagents capable of detecting leukemia cells (e.g., in radioscintigraphic imaging methods).

A wide range of host vector systems suitable for the expression of mutant proteins or fragments thereof are available, see for example, Sambrook et al., 1989, supra; Current Protocols in Molecular Biology, 1995, supra). Vectors for mammalian expression include, for example, pcDNA 3.1 myc-His-tag (Invitrogen) and the retroviral vector pSR.alpha.tkneo (Muller et al., 1991, MCB 11:1785). Using these expression vectors, the polypeptides of the present invention can be preferably expressed in cell lines, including for example CHO COS, 293, 293T, rat-1, 3T3 etc. The host vector systems of the invention are useful for the production of a mutant protein or fragment thereof. Such host-vector systems can be employed to study the functional properties of the proteins.

The present invention provides antibodies that can specifically bind with the polypeptides of the present invention. The term “antibody” is used in the broadest sense and specifically covers monoclonal antibodies, polyclonal antibodies, antibody compositions with polyepitopic specificity, bispecific antibodies, diabodies, chimeric, single-chain, and humanized antibodies, as well as antibody fragments (e.g., Fab, F(ab′)₂, and Fv), so long as they exhibit the desired biological activity. Antibodies can be labeled for use in biological assays (e.g. radioisotope labels, fluorescent labels) to aid in detection of the antibody.

Antibodies that bind to mutant polypeptides can be prepared using, for example, intact polypeptides or fragments containing small peptides of interest, which can be prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, for example, bovine serum albumin (BSA), keyhole limpet hemocyanin (KLH), and thyroglobulin. The coupled peptide is then used to immunize the animal (e.g, a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein can induce the production of antibodies which bind specifically to a given region or three-dimensional structure on the protein; each of these regions or structures is referred to as an antigenic determinant. An antigenic determinant can compete with the intact antigen (i.e., the immunogen used to elicit the immune response) for binding to an antibody.

The phrase “specifically binds to” refers to a binding reaction which is determinative of the presence of a target in the presence of a heterogeneous population of other biologics. Thus, under designated assay conditions, the specified binding region bind preferentially to a particular target and do not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background. For purposes of the present invention, compounds, for example small molecules, can be considered for their ability to specifically bind to mutants described herein.

### Additional BCR/ABL Mutants of the Present Invention

In alternative embodiments, a BCR/ABL mutant comprising one or more of the following amino acid substitutions is encompassed by the present invention: wherein M1 is substituted with either an A, C, D, E, F, G, H, I, K, L, N, P, Q, R, S, T, V, W, or Y; wherein L2 is substituted with either an A, C, D, E, F, G, H, I, K, M, N, P, Q, R, S, T, V, W, or Y; wherein E3 is substituted with either an A, C, D, F, G, H, I, K, L, M, N, P, Q, R, S, T, V, W, or Y; wherein 14 is substituted with either an A, C, D, E, F, G, H, K, L, M, N, P, Q, R, S, T, V, W, or Y; wherein C5 is substituted with either an A, D, E, F, G, H, I, K, L, M, N, P, Q, R, S, T, V, W, or Y; wherein L6 is substituted with either an A, C, D, E, F, G, H, I, K, M, N, P, Q, R, S, T, V, W, or Y; wherein K7 is substituted with either an A, C, D, E, F, G, H, I, L, M, N, P, Q, R, S, T, V, W, or Y; wherein L8 is substituted with either an A, C, D, E, F, G, H, I, K, M, N, P, Q, R, S, T, V, W, or Y; wherein V9 is substituted with either an A, C, D, E, F, G, H, I, K, L, M, N, P, Q, R, S, T, W, or Y; wherein G10 is substituted with either an A, C, D, E, F, H, I, K, L, M, N, P, Q, R, S, T, V, W, or Y; wherein C11 is substituted with either an A, D, E, F, G, H, I, K, L, M, N, P, Q, R, S, T, V, W, or Y; wherein K12 is substituted with either an A, C, D, E, F, G, H, I, L, M, N, P, Q, R, S, T, V, W, or Y; wherein S13 is substituted with either an A, C, D, E, F, G, H, I, K, L, M, N, P, Q, R, T, V, W, or Y; wherein K14 is substituted with either an A, C, D, E, F, G, H, I, L, M, N, P, Q, R, S, T, V, W, or Y; wherein K15 is substituted with either an A, C, D, E, F, G, H, I, L, M, N, P, Q, R, S, T, V, W, or Y; wherein G16 is substituted with either an A, C, D, E, F, H, I, K, L, M, N, P, Q, R, S, T, V, W, or Y; wherein L17 is substituted with either an A, C, D, E, F, G, H, I, K, M, N, P, Q, R, S, T, V, W, or Y; wherein S18 is substituted with either an A, C, D, E, F, G, H, I, K, L, M, N, P, Q, R, T, V, W, or Y; wherein S19 is substituted with either an A, C, D, E, F, G, H, I, K, L, M, N, P, Q, R, T, V, W, or Y; wherein S20 is substituted with either an A, C, D, E, F, G, H, I, K, L, M, N, P, Q, R, T, V, W, or Y; wherein S21 is substituted with either an A, C, D, E, F, G, H, I, K, L, M, N, P, Q, R, T, V, W, or Y; wherein S22 is substituted with either an A, C, D, E, F, G, H, I, K, L, M, N, P, Q, R, T, V, W, or Y; wherein C23 is substituted with either an A, D, E, F, G, H, I, K, L, M, N, P, Q, R, S, T, V, W, or Y; wherein Y24 is substituted with either an A, C, D, E, F, G, H, I, K, L, M, N, P, Q, R, S, T, V, or W; wherein L25 is substituted with either an A, C, D, E, F, G, H, I, K, M, N, P, Q, R, S, T, V, W, or Y; wherein E26 is substituted with either an A, C, D, F, G, H, I, K, L, M, N, P, Q, R, S, T, V, W, or Y; wherein E27 is substituted with either an A, C, D, F, G, H, I, K, L, M, N, P, Q, R, S, T, V, W, or Y; wherein A28 is substituted with either a C, D, E, F, G, H, I, K, L, M, N, P, Q, R, S, T, V, W, or Y; wherein L29 is substituted with either an A, C, D, E, F, G, H, I, K, M, N, P, Q, R, S, T, V, W, or Y; wherein Q30 is substituted with either an A, C, D, E, F, G, H, I, K, L, M, N, P, R, S, T, V, W, or Y; wherein R31 is substituted with either an A, C, D, E, F, G, H, I, K, L, M, N, P, Q, S, T, V, W, or Y; wherein P32 is substituted with either an A, C, D, E, F, G, H, I, K, L, M, N, Q, R, S, T, V, W, or Y; wherein V33 is substituted with either an A, C, D, E, F, G, H, I, K, L, M, N, P, Q, R, S, T, W, or Y; wherein A34 is substituted with either a C, D, E, F, G, H, I, K, L, M, N, P, Q, R, S, T, V, W, or Y; wherein S35 is substituted with either an A, C, D, E, F, G, H, I, K, L, M, N, P, Q, R, T, V, W, or Y; wherein D36 is substituted with either an A, C, E, F, G, H, I, K, L, M, N, P, Q, R, S, T, V, W, or Y; wherein F37 is substituted with either an A, C, D, E, G, H, I, K, L, M, N, P, Q, R, S, T, V, W, or Y; wherein E38 is substituted with either an A, C, D, F, G, H, I, K, L, M, N, P, Q, R, S, T, V, W, or Y; wherein P39 is substituted with either an A, C, D, E, F, G, H, I, K, L, M, N, Q, R, S, T, V, W, or Y; wherein Q40 is substituted with either an A, C, D, E, F, G, H, I, K, L, M, N, P, R, S, T, V, W, or Y; wherein G41 is substituted with either an A, C, D, E, F, H, I, K, L, M, N, P, Q, R, S, T, V, W, or Y; wherein L42 is substituted with either an A, C, D, E, F, G, H, I, K, M, N, P, Q, R, S, T, V, W, or Y; wherein S43 is substituted with either an A, C, D, E, F, G, H, I, K, L, M, N, P, Q, R, T, V, W, or Y; wherein E44 is substituted with either an A, C, D, F, G, H, I, K, L, M, N, P, Q, R, S, T, V, W, or Y; wherein A45 is substituted with either a C, D, E, F, G, H, I, K, L, M, N, P, Q, R, S, T, V, W, or Y; wherein A46 is substituted with either a C, D, E, F, G, H, I, K, L, M, N, P, Q, R, S, T, V, W, or Y; wherein R47 is substituted with either an A, C, D, E, F, G, H, I, K, L, M, N, P, Q, S, T, V, W, or Y; wherein W48 is substituted with either an A, C, D, E, F, G, H, I, K, L, M, N, P, Q, R, S, T, V, or Y; wherein N49 is substituted with either an A, C, D, E, F, G, H, I, K, L, M, P, Q, R, S, T, V, W, or Y; wherein S50 is substituted with either an A, C, D, E, F, G, H, I, K, L, M, N, P, Q, R, T, V, W, or Y; wherein K51 is substituted with either an A, C, D, E, F, G, H, I, L, M, N, P, Q, R, S, T, V, W, or Y; wherein E52 is substituted with either an A, C, D, F, G, H, I, K, L, M, N, P, Q, R, S, T, V, W, or Y; wherein N53 is substituted with either an A, C, D, E, F, G, H, I, K, L, M, P, Q, R, S, T, V, W, or Y; wherein L54 is substituted with either an A, C, D, E, F, G, H, I, K, M, N, P, Q, R, S, T, V, W, or Y; wherein L55 is substituted with either an A, C, D, E, F, G, H, I, K, M, N, P, Q, R, S, T, V, W, or Y; wherein A56 is substituted with either a C, D, E, F, G, H, I, K, L, M, N, P, Q, R, S, T, V, W, or Y; wherein G57 is substituted with either an A, C, D, E, F, H, I, K, L, M, N, P, Q, R, S, T, V, W, or Y; wherein P58 is substituted with either an A, C, D, E, F, G, H, I, K, L, M, N, Q, R, S, T, V, W, or Y; wherein S59 is substituted with either an A, C, D, E, F, G, H, I, K, L, M, N, P, Q, R, T, V, W, or Y; wherein E60 is substituted with either an A, C, D, F, G, H, I, K, L, M, N, P, Q, R, S, T, V, W, or Y; wherein N61 is substituted with either an A, C, D, E, F, G, H, I, K, L, M, P, Q, R, S, T, V, W, or Y; wherein D62 is substituted with either an A, C, E, F, G, H, I, K, L, M, N, P, Q, R, S, T, V, W, or Y; wherein P63 is substituted with either an A, C, D, E, F, G, H, I, K, L, M, N, Q, R, S, T, V, W, or Y; wherein N64 is substituted with either an A, C, D, E, F, G, H, I, K, L, M, P, Q, R, S, T, V, W, or Y; wherein L65 is substituted with either an A, C, D, E, F, G, H, I, K, M, N, P, Q, R, S, T, V, W, or Y; wherein F66 is substituted with either an A, C, D, E, G, H, I, K, L, M, N, P, Q, R, S, T, V, W, or Y; wherein V67 is substituted with either an A, C, D, E, F, G, H, I, K, L, M, N, P, Q, R, S, T, W, or Y; wherein A68 is substituted with either a C, D, E, F, G, H, I, K, L, M, N, P, Q, R, S, T, V, W, or Y; wherein L69 is substituted with either an A, C, D, E, F, G, H, I, K, M, N, P, Q, R, S, T, V, W, or Y; wherein Y70 is substituted with either an A, C, D, E, F, G, H, I, K, L, M, N, P, Q, R, S, T, V, or W; wherein D71 is substituted with either an A, C, E, F, G, H, I, K, L, M, N, P, Q, R, S, T, V, W, or Y; wherein F72 is substituted with either an A, C, D, E, G, H, I, K, L, M, N, P, Q, R, S, T, V, W, or Y; wherein V73 is substituted with either an A, C, D, E, F, G, H, I, K, L, M, N, P, Q, R, S, T, W, or Y; wherein A74 is substituted with either a C, D, E, F, G, H, I, K, L, M, N, P, Q, R, S, T, V, W, or Y; wherein S75 is substituted with either an A, C, D, E, F, G, H, I, K, L, M, N, P, Q, R, T, V, W, or Y; wherein G76 is substituted with either an A, C, D, E, F, H, I, K, L, M, N, P, Q, R, S, T, V, W, or Y; wherein D77 is substituted with either an A, C, E, F, G, H, I, K, L, M, N, P, Q, R, S, T, V, W, or Y; wherein N78 is substituted with either an A, C, D, E, F, G, H, I, K, L, M, P, Q, R, S, T, V, W, or Y; wherein T79 is substituted with either an A, C, D, E, F, G, H, I, K, L, M, N, P, Q, R, S, V, W, or Y; wherein L80 is substituted with either an A, C, D, E, F, G, H, I, K, M, N, P, Q, R, S, T, V, W, or Y; wherein S81 is substituted with either an A, C, D, E, F, G, H, I, K, L, M, N, P, Q, R, T, V, W, or Y; wherein 182 is substituted with either an A, C, D, E, F, G, H, K, L, M, N, P, Q, R, S, T, V, W, or Y; wherein T83 is substituted with either an A, C, D, E, F, G, H, I, K, L, M, N, P, Q, R, S, V, W, or Y; wherein K84 is substituted with either an A, C, D, E, F, G, H, I, L, M, N, P, Q, R, S, T, V, W, or Y; wherein G85 is substituted with either an A, C, D, E, F, H, I, K, L, M, N, P, Q, R, S, T, V, W, or Y; wherein E86 is substituted with either an A, C, D, F, G, H, I, K, L, M, N, P, Q, R, S, T, V, W, or Y; wherein K87 is substituted with either an A, C, D, E, F, G, H, I, L, M, N, P, Q, R, S, T, V, W, or Y; wherein L88 is substituted with either an A, C, D, E, F, G, H, I, K, M, N, P, Q, R, S, T, V, W, or Y; wherein R89 is substituted with either an A, C, D, E, F, G, H, I, K, L, M, N, P, Q, S, T, V, W, or Y; wherein V90 is substituted with either an A, C, D, E, F, G, H, I, K, L, M, N, P, Q, R, S, T, W, or Y; wherein L91 is substituted with either an A, C, D, E, F, G, H, I, K, M, N, P, Q, R, S, T, V, W, or Y; wherein G92 is substituted with either an A, C, D, E, F, H, I, K, L, M, N, P, Q, R, S, T, V, W, or Y; wherein Y93 is substituted with either an A, C, D, E, F, G, H, I, K, L, M, N, P, Q, R, S, T, V, or W; wherein N94 is substituted with either an A, C, D, E, F, G, H, I, K, L, M, P, Q, R, S, T, V, W, or Y; wherein H95 is substituted with either an A, C, D, E, F, G, I, K, L, M, N, P, Q, R, S, T, V, W, or Y; wherein N96 is substituted with either an A, C, D, E, F, G, H, I, K, L, M, P, Q, R, S, T, V, W, or Y; wherein G97 is substituted with either an A, C, D, E, F, H, I, K, L, M, N, P, Q, R, S, T, V, W, or Y; wherein E98 is substituted with either an A, C, D, F, G, H, I, K, L, M, N, P, Q, R, S, T, V, W, or Y; wherein W99 is substituted with either an A, C, D, E, F, G, H, I, K, L, M, N, P, Q, R, S, T, V, or Y; wherein C100 is substituted with either an A, D, E, F, G, H, I, K, L, M, N, P, Q, R, S, T, V, W, or Y; wherein E101 is substituted with either an A, C, D, F, G, H, I, K, L, M, N, P, Q, R, S, T, V, W, or Y; wherein A102 is substituted with either a C, D, E, F, G, H, I, K, L, M, N, P, Q, R, S, T, V, W, or Y; wherein Q103 is substituted with either an A, C, D, E, F, G, H, I, K, L, M, N, P, R, S, T, V, W, or Y; wherein T104 is substituted with either an A, C, D, E, F, G, H, I, K, L, M, N, P, Q, R, S, V, W, or Y; wherein K105 is substituted with either an A, C, D, E, F, G, H, I, L, M, N, P, Q, R, S, T, V, W, or Y; wherein N106 is substituted with either an A, C, D, E, F, G, H, I, K, L, M, P, Q, R, S, T, V, W, or Y; wherein G107 is substituted with either an A, C, D, E, F, H, I, K, L, M, N, P, Q, R, S, T, V, W, or Y; wherein Q108 is substituted with either an A, C, D, E, F, G, H, I, K, L, M, N, P, R, S, T, V, W, or Y; wherein G109 is substituted with either an A, C, D, E, F, H, I, K, L, M, N, P, Q, R, S, T, V, W, or Y; wherein W110 is substituted with either an A, C, D, E, F, G, H, I, K, L, M, N, P, Q, R, S, T, V, or Y; wherein V111 is substituted with either an A, C, D, E, F, G, H, I, K, L, M, N, P, Q, R, S, T, W, or Y; wherein P112 is substituted with either an A, C, D, E, F, G, H, I, K, L, M, N, Q, R, S, T, V, W, or Y; wherein S113 is substituted with either an A, C, D, E, F, G, H, I, K, L, M, N, P, Q, R, T, V, W, or Y; wherein N114 is substituted with either an A, C, D, E, F, G, H, I, K, L, M, P, Q, R, S, T, V, W, or Y; wherein Y115 is substituted with either an A, C, D, E, F, G, H, I, K, L, M, N, P, Q, R, S, T, V, or W; wherein I116 is substituted with either an A, C, D, E, F, G, H, K, L, M, N, P, Q, R, S, T, V, W, or Y; wherein T117 is substituted with either an A, C, D, E, F, G, H, I, K, L, M, N, P, Q, R, S, V, W, or Y; wherein P118 is substituted with either an A, C, D, E, F, G, H, I, K, L, M, N, Q, R, S, T, V, W, or Y; wherein V119 is substituted with either an A, C, D, E, F, G, H, I, K, L, M, N, P, Q, R, S, T, W, or Y; wherein N120 is substituted with either an A, C, D, E, F, G, H, I, K, L, M, P, Q, R, S, T, V, W, or Y; wherein S121 is substituted with either an A, C, D, E, F, G, H, I, K, L, M, N, P, Q, R, T, V, W, or Y; wherein L122 is substituted with either an A, C, D, E, F, G, H, I, K, M, N, P, Q, R, S, T, V, W, or Y; wherein E123 is substituted with either an A, C, D, F, G, H, I, K, L, M, N, P, Q, R, S, T, V, W, or Y; wherein K124 is substituted with either an A, C, D, E, F, G, H, I, L, M, N, P, Q, R, S, T, V, W, or Y; wherein H125 is substituted with either an A, C, D, E, F, G, I, K, L, M, N, P, Q, R, S, T, V, W, or Y; wherein S126 is substituted with either an A, C, D, E, F, G, H, I, K, L, M, N, P, Q, R, T, V, W, or Y; wherein W127 is substituted with either an A, C, D, E, F, G, H, I, K, L, M, N, P, Q, R, S, T, V, or Y; wherein Y128 is substituted with either an A, C, D, E, F, G, H, I, K, L, M, N, P, Q, R, S, T, V, or W; wherein H129 is substituted with either an A, C, D, E, F, G, I, K, L, M, N, P, Q, R, S, T, V, W, or Y; wherein G130 is substituted with either an A, C, D, E, F, H, I, K, L, M, N, P, Q, R, S, T, V, W, or Y; wherein P131 is substituted with either an A, C, D, E, F, G, H, I, K, L, M, N, Q, R, S, T, V, W, or Y; wherein V132 is substituted with either an A, C, D, E, F, G, H, I, K, L, M, N, P, Q, R, S, T, W, or Y; wherein S133 is substituted with either an A, C, D, E, F, G, H, I, K, L, M, N, P, Q, R, T, V, W, or Y; wherein R134 is substituted with either an A, C, D, E, F, G, H, I, K, L, M, N, P, Q, S, T, V, W, or Y; wherein N135 is substituted with either an A, C, D, E, F, G, H, I, K, L, M, P, Q, R, S, T, V, W, or Y; wherein A136 is substituted with either a C, D, E, F, G, H, I, K, L, M, N, P, Q, R, S, T, V, W, or Y; wherein A137 is substituted with either a C, D, E, F, G, H, I, K, L, M, N, P, Q, R, S, T, V, W, or Y; wherein E138 is substituted with either an A, C, D, F, G, H, I, K, L, M, N, P, Q, R, S, T, V, W, or Y; wherein Y139 is substituted with either an A, C, D, E, F, G, H, I, K, L, M, N, P, Q, R, S, T, V, or W; wherein P140 is substituted with either an A, C, D, E, F, G, H, I, K, L, M, N, Q, R, S, T, V, W, or Y; wherein L141 is substituted with either an A, C, D, E, F, G, H, I, K, M, N, P, Q, R, S, T, V, W, or Y; wherein S142 is substituted with either an A, C, D, E, F, G, H, I, K, L, M, N, P, Q, R, T, V, W, or Y; wherein S143 is substituted with either an A, C, D, E, F, G, H, I, K, L, M, N, P, Q, R, T, V, W, or Y; wherein G144 is substituted with either an A, C, D, E, F, H, I, K, L, M, N, P, Q, R, S, T, V, W, or Y; wherein I145 is substituted with either an A, C, D, E, F, G, H, K, L, M, N, P, Q, R, S, T, V, W, or Y; wherein N146 is substituted with either an A, C, D, E, F, G, H, I, K, L, M, P, Q, R, S, T, V, W, or Y; wherein G147 is substituted with either an A, C, D, E, F, H, I, K, L, M, N, P, Q, R, S, T, V, W, or Y; wherein S148 is substituted with either an A, C, D, E, F, G, H, I, K, L, M, N, P, Q, R, T, V, W, or Y; wherein F149 is substituted with either an A, C, D, E, G, H, I, K, L, M, N, P, Q, R, S, T, V, W, or Y; wherein L150 is substituted with either an A, C, D, E, F, G, H, I, K, M, N, P, Q, R, S, T, V, W, or Y; wherein V151 is substituted with either an A, C, D, E, F, G, H, I, K, L, M, N, P, Q, R, S, T, W, or Y; wherein R152 is substituted with either an A, C, D, E, F, G, H, I, K, L, M, N, P, Q, S, T, V, W, or Y; wherein E153 is substituted with either an A, C, D, F, G, H, I, K, L, M, N, P, Q, R, S, T, V, W, or Y; wherein S154 is substituted with either an A, C, D, E, F, G, H, I, K, L, M, N, P, Q, R, T, V, W, or Y; wherein E155 is substituted with either an A, C, D, F, G, H, I, K, L, M, N, P, Q, R, S, T, V, W, or Y; wherein S156 is substituted with either an A, C, D, E, F, G, H, I, K, L, M, N, P, Q, R, T, V, W, or Y; wherein S157 is substituted with either an A, C, D, E, F, G, H, I, K, L, M, N, P, Q, R, T, V, W, or Y; wherein P158 is substituted with either an A, C, D, E, F, G, H, I, K, L, M, N, Q, R, S, T, V, W, or Y; wherein S159 is substituted with either an A, C, D, E, F, G, H, I, K, L, M, N, P, Q, R, T, V, W, or Y; wherein Q160 is substituted with either an A, C, D, E, F, G, H, I, K, L, M, N, P, R, S, T, V, W, or Y; wherein R161 is substituted with either an A, C, D, E, F, G, H, I, K, L, M, N, P, Q, S, T, V, W, or Y; wherein S162 is substituted with either an A, C, D, E, F, G, H, I, K, L, M, N, P, Q, R, T, V, W, or Y; wherein I163 is substituted with either an A, C, D, E, F, G, H, K, L, M, N, P, Q, R, S, T, V, W, or Y; wherein S164 is substituted with either an A, C, D, E, F, G, H, I, K, L, M, N, P, Q, R, T, V, W, or Y; wherein L165 is substituted with either an A, C, D, E, F, G, H, I, K, M, N, P, Q, R, S, T, V, W, or Y; wherein R166 is substituted with either an A, C, D, E, F, G, H, I, K, L, M, N, P, Q, S, T, V, W, or Y; wherein Y167 is substituted with either an A, C, D, E, F, G, H, I, K, L, M, N, P, Q, R, S, T, V, or W; wherein E168 is substituted with either an A, C, D, F, G, H, I, K, L, M, N, P, Q, R, S, T, V, W, or Y; wherein G169 is substituted with either an A, C, D, E, F, H, I, K, L, M, N, P, Q, R, S, T, V, W, or Y; wherein R170 is substituted with either an A, C, D, E, F, G, H, I, K, L, M, N, P, Q, S, T, V, W, or Y; wherein V171 is substituted with either an A, C, D, E, F, G, H, I, K, L, M, N, P, Q, R, S, T, W, or Y; wherein Y172 is substituted with either an A, C, D, E, F, G, H, I, K, L, M, N, P, Q, R, S, T, V, or W; wherein H173 is substituted with either an A, C, D, E, F, G, I, K, L, M, N, P, Q, R, S, T, V, W, or Y; wherein Y174 is substituted with either an A, C, D, E, F, G, H, I, K, L, M, N, P, Q, R, S, T, V, or W; wherein R175 is substituted with either an A, C, D, E, F, G, H, I, K, L, M, N, P, Q, S, T, V, W, or Y; wherein 176 is substituted with either an A, C, D, E, F, G, H, K, L, M, N, P, Q, R, S, T, V, W, or Y; wherein N177 is substituted with either an A, C, D, E, F, G, H, I, K, L, M, P, Q, R, S, T, V, W, or Y; wherein T178 is substituted with either an A, C, D, E, F, G, H, I, K, L, M, N, P, Q, R, S, V, W, or Y; wherein A179 is substituted with either a C, D, E, F, G, H, I, K, L, M, N, P, Q, R, S, T, V, W, or Y; wherein S180 is substituted with either an A, C, D, E, F, G, H, I, K, L, M, N, P, Q, R, T, V, W, or Y; wherein D181 is substituted with either an A, C, E, F, G, H, I, K, L, M, N, P, Q, R, S, T, V, W, or Y; wherein G182 is substituted with either an A, C, D, E, F, H, I, K, L, M, N, P, Q, R, S, T, V, W, or Y; wherein K183 is substituted with either an A, C, D, E, F, G, H, I, L, M, N, P, Q, R, S, T, V, W, or Y; wherein L184 is substituted with either an A, C, D, E, F, G, H, I, K, M, N, P, Q, R, S, T, V, W, or Y; wherein Y185 is substituted with either an A, C, D, E, F, G, H, I, K, L, M, N, P, Q, R, S, T, V, or W; wherein V186 is substituted with either an A, C, D, E, F, G, H, I, K, L, M, N, P, Q, R, S, T, W, or Y; wherein S187 is substituted with either an A, C, D, E, F, G, H, I, K, L, M, N, P, Q, R, T, V, W, or Y; wherein S188 is substituted with either an A, C, D, E, F, G, H, I, K, L, M, N, P, Q, R, T, V, W, or Y; wherein E189 is substituted with either an A, C, D, F, G, H, I, K, L, M, N, P, Q, R, S, T, V, W, or Y; wherein S190 is substituted with either an A, C, D, E, F, G, H, I, K, L, M, N, P, Q, R, T, V, W, or Y; wherein R191 is substituted with either an A, C, D, E, F, G, H, I, K, L, M, N, P, Q, S, T, V, W, or Y; wherein F192 is substituted with either an A, C, D, E, G, H, I, K, L, M, N, P, Q, R, S, T, V, W, or Y; wherein N193 is substituted with either an A, C, D, E, F, G, H, I, K, L, M, P, Q, R, S, T, V, W, or Y; wherein T194 is substituted with either an A, C, D, E, F, G, H, I, K, L, M, N, P, Q, R, S, V, W, or Y; wherein L195 is substituted with either an A, C, D, E, F, G, H, I, K, M, N, P, Q, R, S, T, V, W, or Y; wherein A196 is substituted with either a C, D, E, F, G, H, I, K, L, M, N, P, Q, R, S, T, V, W, or Y; wherein E197 is substituted with either an A, C, D, F, G, H, I, K, L, M, N, P, Q, R, S, T, V, W, or Y; wherein L198 is substituted with either an A, C, D, E, F, G, H, I, K, M, N, P, Q, R, S, T, V, W, or Y; wherein V199 is substituted with either an A, C, D, E, F, G, H, I, K, L, M, N, P, Q, R, S, T, W, or Y; wherein H200 is substituted with either an A, C, D, E, F, G, I, K, L, M, N, P, Q, R, S, T, V, W, or Y; wherein H201 is substituted with either an A, C, D, E, F, G, I, K, L, M, N, P, Q, R, S, T, V, W, or Y; wherein H202 is substituted with either an A, C, D, E, F, G, I, K, L, M, N, P, Q, R, S, T, V, W, or Y; wherein S203 is substituted with either an A, C, D, E, F, G, H, I, K, L, M, N, P, Q, R, T, V, W, or Y; wherein T204 is substituted with either an A, C, D, E, F, G, H, I, K, L, M, N, P, Q, R, S, V, W, or Y; wherein V205 is substituted with either an A, C, D, E, F, G, H, I, K, L, M, N, P, Q, R, S, T, W, or Y; wherein A206 is substituted with either a C, D, E, F, G, H, I, K, L, M, N, P, Q, R, S, T, V, W, or Y; wherein D207 is substituted with either an A, C, E, F, G, H, I, K, L, M, N, P, Q, R, S, T, V, W, or Y; wherein G208 is substituted with either an A, C, D, E, F, H, I, K, L, M, N, P, Q, R, S, T, V, W, or Y; wherein L209 is substituted with either an A, C, D, E, F, G, H, I, K, M, N, P, Q, R, S, T, V, W, or Y; wherein I210 is substituted with either an A, C, D, E, F, G, H, K, L, M, N, P, Q, R, S, T, V, W, or Y; wherein T211 is substituted with either an A, C, D, E, F, G, H, I, K, L, M, N, P, Q, R, S, V, W, or Y; wherein T212 is substituted with either an A, C, D, E, F, G, H, I, K, L, M, N, P, Q, R, S, V, W, or Y; wherein L213 is substituted with either an A, C, D, E, F, G, H, I, K, M, N, P, Q, R, S, T, V, W, or Y; wherein H214 is substituted with either an A, C, D, E, F, G, I, K, L, M, N, P, Q, R, S, T, V, W, or Y; wherein Y215 is substituted with either an A, C, D, E, F, G, H, I, K, L, M, N, P, Q, R, S, T, V, or W; wherein P216 is substituted with either an A, C, D, E, F, G, H, I, K, L, M, N, Q, R, S, T, V, W, or Y; wherein A217 is substituted with either a C, D, E, F, G, H, I, K, L, M, N, P, Q, R, S, T, V, W, or Y; wherein P218 is substituted with either an A, C, D, E, F, G, H, I, K, L, M, N, Q, R, S, T, V, W, or Y; wherein K219 is substituted with either an A, C, D, E, F, G, H, I, L, M, N, P, Q, R, S, T, V, W, or Y; wherein R220 is substituted with either an A, C, D, E, F, G, H, I, K, L, M, N, P, Q, S, T, V, W, or Y; wherein N221 is substituted with either an A, C, D, E, F, G, H, I, K, L, M, P, Q, R, S, T, V, W, or Y; wherein K222 is substituted with either an A, C, D, E, F, G, H, I, L, M, N, P, Q, R, S, T, V, W, or Y; wherein P223 is substituted with either an A, C, D, E, F, G, H, I, K, L, M, N, Q, R, S, T, V, W, or Y; wherein T224 is substituted with either an A, C, D, E, F, G, H, I, K, L, M, N, P, Q, R, S, V, W, or Y; wherein V225 is substituted with either an A, C, D, E, F, G, H, I, K, L, M, N, P, Q, R, S, T, W, or Y; wherein Y226 is substituted with either an A, C, D, E, F, G, H, I, K, L, M, N, P, Q, R, S, T, V, or W; wherein G227 is substituted with either an A, C, D, E, F, H, I, K, L, M, N, P, Q, R, S, T, V, W, or Y; wherein V228 is substituted with either an A, C, D, E, F, G, H, I, K, L, M, N, P, Q, R, S, T, W, or Y; wherein S229 is substituted with either an A, C, D, E, F, G, H, I, K, L, M, N, P, Q, R, T, V, W, or Y; wherein P230 is substituted with either an A, C, D, E, F, G, H, I, K, L, M, N, Q, R, S, T, V, W, or Y; wherein N231 is substituted with either an A, C, D, E, F, G, H, I, K, L, M, P, Q, R, S, T, V, W, or Y; wherein Y232 is substituted with either an A, C, D, E, F, G, H, I, K, L, M, N, P, Q, R, S, T, V, or W; wherein D233 is substituted with either an A, C, E, F, G, H, I, K, L, M, N, P, Q, R, S, T, V, W, or Y; wherein K234 is substituted with either an A, C, D, E, F, G, H, I, L, M, N, P, Q, R, S, T, V, W, or Y; wherein W235 is substituted with either an A, C, D, E, F, G, H, I, K, L, M, N, P, Q, R, S, T, V, or Y; wherein E236 is substituted with either an A, C, D, F, G, H, I, K, L, M, N, P, Q, R, S, T, V, W, or Y; wherein M237 is substituted with either an A, C, D, E, F, G, H, I, K, L, N, P, Q, R, S, T, V, W, or Y; wherein E238 is substituted with either an A, C, D, F, G, H, I, K, L, M, N, P, Q, R, S, T, V, W, or Y; wherein R239 is substituted with either an A, C, D, E, F, G, H, I, K, L, M, N, P, Q, S, T, V, W, or Y; wherein T240 is substituted with either an A, C, D, E, F, G, H, I, K, L, M, N, P, Q, R, S, V, W, or Y; wherein D241 is substituted with either an A, C, E, F, G, H, I, K, L, M, N, P, Q, R, S, T, V, W, or Y; wherein I242 is substituted with either an A, C, D, E, F, G, H, K, L, M, N, P, Q, R, S, T, V, W, or Y; wherein T243 is substituted with either an A, C, D, E, F, G, H, I, K, L, M, N, P, Q, R, S, V, W, or Y; wherein M244 is substituted with either an A, C, D, E, F, G, H, I, K, L, N, P, Q, R, S, T, V, W, or Y; wherein K245 is substituted with either an A, C, D, E, F, G, H, I, L, M, N, P, Q, R, S, T, V, W, or Y; wherein H246 is substituted with either an A, C, D, E, F, G, I, K, L, M, N, P, Q, R, S, T, V, W, or Y; wherein K247 is substituted with either an A, C, D, E, F, G, H, I, L, M, N, P, Q, R, S, T, V, W, or Y; wherein L248 is substituted with either an A, C, D, E, F, G, H, I, K, M, N, P, Q, R, S, T, V, W, or Y; wherein G249 is substituted with either an A, C, D, E, F, H, I, K, L, M, N, P, Q, R, S, T, V, W, or Y; wherein G250 is substituted with either an A, C, D, E, F, H, I, K, L, M, N, P, Q, R, S, T, V, W, or Y; wherein G251 is substituted with either an A, C, D, E, F, H, I, K, L, M, N, P, Q, R, S, T, V, W, or Y; wherein Q252 is substituted with either an A, C, D, E, F, G, H, I, K, L, M, N, P, R, S, T, V, W, or Y; wherein Y253 is substituted with either an A, C, D, E, F, G, H, I, K, L, M, N, P, Q, R, S, T, V, or W; wherein G254 is substituted with either an A, C, D, E, F, H, I, K, L, M, N, P, Q, R, S, T, V, W, or Y; wherein E255 is substituted with either an A, C, D, F, G, H, I, K, L, M, N, P, Q, R, S, T, V, W, or Y; wherein V256 is substituted with either an A, C, D, E, F, G, H, I, K, L, M, N, P, Q, R, S, T, W, or Y; wherein Y257 is substituted with either an A, C, D, E, F, G, H, I, K, L, M, N, P, Q, R, S, T, V, or W; wherein E258 is substituted with either an A, C, D, F, G, H, I, K, L, M, N, P, Q, R, S, T, V, W, or Y; wherein G259 is substituted with either an A, C, D, E, F, H, I, K, L, M, N, P, Q, R, S, T, V, W, or Y; wherein V260 is substituted with either an A, C, D, E, F, G, H, I, K, L, M, N, P, Q, R, S, T, W, or Y; wherein W261 is substituted with either an A, C, D, E, F, G, H, I, K, L, M, N, P, Q, R, S, T, V, or Y; wherein K262 is substituted with either an A, C, D, E, F, G, H, I, L, M, N, P, Q, R, S, T, V, W, or Y; wherein K263 is substituted with either an A, C, D, E, F, G, H, I, L, M, N, P, Q, R, S, T, V, W, or Y; wherein Y264 is substituted with either an A, C, D, E, F, G, H, I, K, L, M, N, P, Q, R, S, T, V, or W; wherein S265 is substituted with either an A, C, D, E, F, G, H, I, K, L, M, N, P, Q, R, T, V, W, or Y; wherein L266 is substituted with either an A, C, D, E, F, G, H, I, K, M, N, P, Q, R, S, T, V, W, or Y; wherein T267 is substituted with either an A, C, D, E, F, G, H, I, K, L, M, N, P, Q, R, S, V, W, or Y; wherein V268 is substituted with either an A, C, D, E, F, G, H, I, K, L, M, N, P, Q, R, S, T, W, or Y; wherein A269 is substituted with either a C, D, E, F, G, H, I, K, L, M, N, P, Q, R, S, T, V, W, or Y; wherein V270 is substituted with either an A, C, D, E, F, G, H, I, K, L, M, N, P, Q, R, S, T, W, or Y; wherein K271 is substituted with either an A, C, D, E, F, G, H, I, L, M, N, P, Q, R, S, T, V, W, or Y; wherein T272 is substituted with either an A, C, D, E, F, G, H, I, K, L, M, N, P, Q, R, S, V, W, or Y; wherein L273 is substituted with either an A, C, D, E, F, G, H, I, K, M, N, P, Q, R, S, T, V, W, or Y; wherein K274 is substituted with either an A, C, D, E, F, G, H, I, L, M, N, P, Q, R, S, T, V, W, or Y; wherein E275 is substituted with either an A, C, D, F, G, H, I, K, L, M, N, P, Q, R, S, T, V, W, or Y; wherein D276 is substituted with either an A, C, E, F, G, H, I, K, L, M, N, P, Q, R, S, T, V, W, or Y; wherein T277 is substituted with either an A, C, D, E, F, G, H, I, K, L, M, N, P, Q, R, S, V, W, or Y; wherein M278 is substituted with either an A, C, D, E, F, G, H, I, K, L, N, P, Q, R, S, T, V, W, or Y; wherein E279 is substituted with either an A, C, D, F, G, H, I, K, L, M, N, P, Q, R, S, T, V, W, or Y; wherein V280 is substituted with either an A, C, D, E, F, G, H, I, K, L, M, N, P, Q, R, S, T, W, or Y; wherein E281 is substituted with either an A, C, D, F, G, H, I, K, L, M, N, P, Q, R, S, T, V, W, or Y; wherein E282 is substituted with either an A, C, D, F, G, H, I, K, L, M, N, P, Q, R, S, T, V, W, or Y; wherein F283 is substituted with either an A, C, D, E, G, H, I, K, L, M, N, P, Q, R, S, T, V, W, or Y; wherein L284 is substituted with either an A, C, D, E, F, G, H, I, K, M, N, P, Q, R, S, T, V, W, or Y; wherein K285 is substituted with either an A, C, D, E, F, G, H, I, L, M, N, P, Q, R, S, T, V, W, or Y; wherein E286 is substituted with either an A, C, D, F, G, H, I, K, L, M, N, P, Q, R, S, T, V, W, or Y; wherein A287 is substituted with either a C, D, E, F, G, H, I, K, L, M, N, P, Q, R, S, T, V, W, or Y; wherein A288 is substituted with either a C, D, E, F, G, H, I, K, L, M, N, P, Q, R, S, T, V, W, or Y; wherein V289 is substituted with either an A, C, D, E, F, G, H, I, K, L, M, N, P, Q, R, S, T, W, or Y; wherein M290 is substituted with either an A, C, D, E, F, G, H, I, K, L, N, P, Q, R, S, T, V, W, or Y; wherein K291 is substituted with either an A, C, D, E, F, G, H, I, L, M, N, P, Q, R, S, T, V, W, or Y; wherein E292 is substituted with either an A, C, D, F, G, H, I, K, L, M, N, P, Q, R, S, T, V, W, or Y; wherein I293 is substituted with either an A, C, D, E, F, G, H, K, L, M, N, P, Q, R, S, T, V, W, or Y; wherein K294 is substituted with either an A, C, D, E, F, G, H, I, L, M, N, P, Q, R, S, T, V, W, or Y; wherein H295 is substituted with either an A, C, D, E, F, G, I, K, L, M, N, P, Q, R, S, T, V, W, or Y; wherein P296 is substituted with either an A, C, D, E, F, G, H, I, K, L, M, N, Q, R, S, T, V, W, or Y; wherein N297 is substituted with either an A, C, D, E, F, G, H, I, K, L, M, P, Q, R, S, T, V, W, or Y; wherein L298 is substituted with either an A, C, D, E, F, G, H, I, K, M, N, P, Q, R, S, T, V, W, or Y; wherein V299 is substituted with either an A, C, D, E, F, G, H, I, K, L, M, N, P, Q, R, S, T, W, or Y; wherein Q300 is substituted with either an A, C, D, E, F, G, H, I, K, L, M, N, P, R, S, T, V, W, or Y; wherein L301 is substituted with either an A, C, D, E, F, G, H, I, K, M, N, P, Q, R, S, T, V, W, or Y; wherein L302 is substituted with either an A, C, D, E, F, G, H, I, K, M, N, P, Q, R, S, T, V, W, or Y; wherein G303 is substituted with either an A, C, D, E, F, H, I, K, L, M, N, P, Q, R, S, T, V, W, or Y; wherein V304 is substituted with either an A, C, D, E, F, G, H, I, K, L, M, N, P, Q, R, S, T, W, or Y; wherein C305 is substituted with either an A, D, E, F, G, H, I, K, L, M, N, P, Q, R, S, T, V, W, or Y; wherein T306 is substituted with either an A, C, D, E, F, G, H, I, K, L, M, N, P, Q, R, S, V, W, or Y; wherein R307 is substituted with either an A, C, D, E, F, G, H, I, K, L, M, N, P, Q, S, T, V, W, or Y; wherein E308 is substituted with either an A, C, D, F, G, H, I, K, L, M, N, P, Q, R, S, T, V, W, or Y; wherein P309 is substituted with either an A, C, D, E, F, G, H, I, K, L, M, N, Q, R, S, T, V, W, or Y; wherein P310 is substituted with either an A, C, D, E, F, G, H, I, K, L, M, N, Q, R, S, T, V, W, or Y; wherein F311 is substituted with either an A, C, D, E, G, H, I, K, L, M, N, P, Q, R, S, T, V, W, or Y; wherein Y312 is substituted with either an A, C, D, E, F, G, H, I, K, L, M, N, P, Q, R, S, T, V, or W; wherein I313 is substituted with either an A, C, D, E, F, G, H, K, L, M, N, P, Q, R, S, T, V, W, or Y; wherein I314 is substituted with either an A, C, D, E, F, G, H, K, L, M, N, P, Q, R, S, T, V, W, or Y; wherein T315 is substituted with either an A, C, D, E, F, G, H, I, K, L, M, N, P, Q, R, S, V, W, or Y; wherein E316 is substituted with either an A, C, D, F, G, H, I, K, L, M, N, P, Q, R, S, T, V, W, or Y; wherein F317 is substituted with either an A, C, D, E, G, H, I, K, L, M, N, P, Q, R, S, T, V, W, or Y; wherein M318 is substituted with either an A, C, D, E, F, G, H, I, K, L, N, P, Q, R, S, T, V, W, or Y; wherein T319 is substituted with either an A, C, D, E, F, G, H, I, K, L, M, N, P, Q, R, S, V, W, or Y; wherein Y320 is substituted with either an A, C, D, E, F, G, H, I, K, L, M, N, P, Q, R, S, T, V, or W; wherein G321 is substituted with either an A, C, D, E, F, H, I, K, L, M, N, P, Q, R, S, T, V, W, or Y; wherein N322 is substituted with either an A, C, D, E, F, G, H, I, K, L, M, P, Q, R, S, T, V, W, or Y; wherein L323 is substituted with either an A, C, D, E, F, G, H, I, K, M, N, P, Q, R, S, T, V, W, or Y; wherein L324 is substituted with either an A, C, D, E, F, G, H, I, K, M, N, P, Q, R, S, T, V, W, or Y; wherein D325 is substituted with either an A, C, E, F, G, H, I, K, L, M, N, P, Q, R, S, T, V, W, or Y; wherein Y326 is substituted with either an A, C, D, E, F, G, H, I, K, L, M, N, P, Q, R, S, T, V, or W; wherein L327 is substituted with either an A, C, D, E, F, G, H, I, K, M, N, P, Q, R, S, T, V, W, or Y; wherein R328 is substituted with either an A, C, D, E, F, G, H, I, K, L, M, N, P, Q, S, T, V, W, or Y; wherein E329 is substituted with either an A, C, D, F, G, H, I, K, L, M, N, P, Q, R, S, T, V, W, or Y; wherein C330 is substituted with either an A, D, E, F, G, H, I, K, L, M, N, P, Q, R, S, T, V, W, or Y; wherein N331 is substituted with either an A, C, D, E, F, G, H, I, K, L, M, P, Q, R, S, T, V, W, or Y; wherein R332 is substituted with either an A, C, D, E, F, G, H, I, K, L, M, N, P, Q, S, T, V, W, or Y; wherein Q333 is substituted with either an A, C, D, E, F, G, H, I, K, L, M, N, P, R, S, T, V, W, or Y; wherein E334 is substituted with either an A, C, D, F, G, H, I, K, L, M, N, P, Q, R, S, T, V, W, or Y; wherein V335 is substituted with either an A, C, D, E, F, G, H, I, K, L, M, N, P, Q, R, S, T, W, or Y; wherein N336 is substituted with either an A, C, D, E, F, G, H, I, K, L, M, P, Q, R, S, T, V, W, or Y; wherein A337 is substituted with either a C, D, E, F, G, H, I, K, L, M, N, P, Q, R, S, T, V, W, or Y; wherein V338 is substituted with either an A, C, D, E, F, G, H, I, K, L, M, N, P, Q, R, S, T, W, or Y; wherein V339 is substituted with either an A, C, D, E, F, G, H, I, K, L, M, N, P, Q, R, S, T, W, or Y; wherein L340 is substituted with either an A, C, D, E, F, G, H, I, K, M, N, P, Q, R, S, T, V, W, or Y; wherein L341 is substituted with either an A, C, D, E, F, G, H, I, K, M, N, P, Q, R, S, T, V, W, or Y; wherein Y342 is substituted with either an A, C, D, E, F, G, H, I, K, L, M, N, P, Q, R, S, T, V, or W; wherein M343 is substituted with either an A, C, D, E, F, G, H, I, K, L, N, P, Q, R, S, T, V, W, or Y; wherein A344 is substituted with either a C, D, E, F, G, H, I, K, L, M, N, P, Q, R, S, T, V, W, or Y; wherein T345 is substituted with either an A, C, D, E, F, G, H, I, K, L, M, N, P, Q, R, S, V, W, or Y; wherein Q346 is substituted with either an A, C, D, E, F, G, H, I, K, L, M, N, P, R, S, T, V, W, or Y; wherein I347 is substituted with either an A, C, D, E, F, G, H, K, L, M, N, P, Q, R, S, T, V, W, or Y; wherein S348 is substituted with either an A, C, D, E, F, G, H, I, K, L, M, N, P, Q, R, T, V, W, or Y; wherein S349 is substituted with either an A, C, D, E, F, G, H, I, K, L, M, N, P, Q, R, T, V, W, or Y; wherein A350 is substituted with either a C, D, E, F, G, H, I, K, L, M, N, P, Q, R, S, T, V, W, or Y; wherein M351 is substituted with either an A, C, D, E, F, G, H, I, K, L, N, P, Q, R, S, T, V, W, or Y; wherein E352 is substituted with either an A, C, D, F, G, H, I, K, L, M, N, P, Q, R, S, T, V, W, or Y; wherein Y353 is substituted with either an A, C, D, E, F, G, H, I, K, L, M, N, P, Q, R, S, T, V, or W; wherein L354 is substituted with either an A, C, D, E, F, G, H, I, K, M, N, P, Q, R, S, T, V, W, or Y; wherein E355 is substituted with either an A, C, D, F, G, H, I, K, L, M, N, P, Q, R, S, T, V, W, or Y; wherein K356 is substituted with either an A, C, D, E, F, G, H, I, L, M, N, P, Q, R, S, T, V, W, or Y; wherein K357 is substituted with either an A, C, D, E, F, G, H, I, L, M, N, P, Q, R, S, T, V, W, or Y; wherein N358 is substituted with either an A, C, D, E, F, G, H, I, K, L, M, P, Q, R, S, T, V, W, or Y; wherein F359 is substituted with either an A, C, D, E, G, H, I, K, L, M, N, P, Q, R, S, T, V, W, or Y; wherein I360 is substituted with either an A, C, D, E, F, G, H, K, L, M, N, P, Q, R, S, T, V, W, or Y; wherein H361 is substituted with either an A, C, D, E, F, G, I, K, L, M, N, P, Q, R, S, T, V, W, or Y; wherein R362 is substituted with either an A, C, D, E, F, G, H, I, K, L, M, N, P, Q, S, T, V, W, or Y; wherein D363 is substituted with either an A, C, E, F, G, H, I, K, L, M, N, P, Q, R, S, T, V, W, or Y; wherein L364 is substituted with either an A, C, D, E, F, G, H, I, K, M, N, P, Q, R, S, T, V, W, or Y; wherein A365 is substituted with either a C, D, E, F, G, H, I, K, L, M, N, P, Q, R, S, T, V, W, or Y; wherein A366 is substituted with either a C, D, E, F, G, H, I, K, L, M, N, P, Q, R, S, T, V, W, or Y; wherein R367 is substituted with either an A, C, D, E, F, G, H, I, K, L, M, N, P, Q, S, T, V, W, or Y; wherein N368 is substituted with either an A, C, D, E, F, G, H, I, K, L, M, P, Q, R, S, T, V, W, or Y; wherein C369 is substituted with either an A, D, E, F, G, H, I, K, L, M, N, P, Q, R, S, T, V, W, or Y; wherein L370 is substituted with either an A, C, D, E, F, G, H, I, K, M, N, P, Q, R, S, T, V, W, or Y; wherein V371 is substituted with either an A, C, D, E, F, G, H, I, K, L, M, N, P, Q, R, S, T, W, or Y; wherein G372 is substituted with either an A, C, D, E, F, H, I, K, L, M, N, P, Q, R, S, T, V, W, or Y; wherein E373 is substituted with either an A, C, D, F, G, H, I, K, L, M, N, P, Q, R, S, T, V, W, or Y; wherein N374 is substituted with either an A, C, D, E, F, G, H, I, K, L, M, P, Q, R, S, T, V, W, or Y; wherein H375 is substituted with either an A, C, D, E, F, G, I, K, L, M, N, P, Q, R, S, T, V, W, or Y; wherein L376 is substituted with either an A, C, D, E, F, G, H, I, K, M, N, P, Q, R, S, T, V, W, or Y; wherein V377 is substituted with either an A, C, D, E, F, G, H, I, K, L, M, N, P, Q, R, S, T, W, or Y; wherein K378 is substituted with either an A, C, D, E, F, G, H, I, L, M, N, P, Q, R, S, T, V, W, or Y; wherein V379 is substituted with either an A, C, D, E, F, G, H, I, K, L, M, N, P, Q, R, S, T, W, or Y; wherein A380 is substituted with either a C, D, E, F, G, H, I, K, L, M, N, P, Q, R, S, T, V, W, or Y; wherein D381 is substituted with either an A, C, E, F, G, H, I, K, L, M, N, P, Q, R, S, T, V, W, or Y; wherein F382 is substituted with either an A, C, D, E, G, H, I, K, L, M, N, P, Q, R, S, T, V, W, or Y; wherein G383 is substituted with either an A, C, D, E, F, H, I, K, L, M, N, P, Q, R, S, T, V, W, or Y; wherein L384 is substituted with either an A, C, D, E, F, G, H, I, K, M, N, P, Q, R, S, T, V, W, or Y; wherein S385 is substituted with either an A, C, D, E, F, G, H, I, K, L, M, N, P, Q, R, T, V, W, or Y; wherein R386 is substituted with either an A, C, D, E, F, G, H, I, K, L, M, N, P, Q, S, T, V, W, or Y; wherein L387 is substituted with either an A, C, D, E, F, G, H, I, K, M, N, P, Q, R, S, T, V, W, or Y; wherein M388 is substituted with either an A, C, D, E, F, G, H, I, K, L, N, P, Q, R, S, T, V, W, or Y; wherein T389 is substituted with either an A, C, D, E, F, G, H, I, K, L, M, N, P, Q, R, S, V, W, or Y; wherein G390 is substituted with either an A, C, D, E, F, H, I, K, L, M, N, P, Q, R, S, T, V, W, or Y; wherein D391 is substituted with either an A, C, E, F, G, H, I, K, L, M, N, P, Q, R, S, T, V, W, or Y; wherein T392 is substituted with either an A, C, D, E, F, G, H, I, K, L, M, N, P, Q, R, S, V, W, or Y; wherein Y393 is substituted with either an A, C, D, E, F, G, H, I, K, L, M, N, P, Q, R, S, T, V, or W; wherein T394 is substituted with either an A, C, D, E, F, G, H, I, K, L, M, N, P, Q, R, S, V, W, or Y; wherein A395 is substituted with either a C, D, E, F, G, H, I, K, L, M, N, P, Q, R, S, T, V, W, or Y; wherein H396 is substituted with either an A, C, D, E, F, G, I, K, L, M, N, P, Q, R, S, T, V, W, or Y; wherein A397 is substituted with either a C, D, E, F, G, H, I, K, L, M, N, P, Q, R, S, T, V, W, or Y; wherein G398 is substituted with either an A, C, D, E, F, H, I, K, L, M, N, P, Q, R, S, T, V, W, or Y; wherein A399 is substituted with either a C, D, E, F, G, H, I, K, L, M, N, P, Q, R, S, T, V, W, or Y; wherein K400 is substituted with either an A, C, D, E, F, G, H, I, L, M, N, P, Q, R, S, T, V, W, or Y; wherein F401 is substituted with either an A, C, D, E, G, H, I, K, L, M, N, P, Q, R, S, T, V, W, or Y; wherein P402 is substituted with either an A, C, D, E, F, G, H, I, K, L, M, N, Q, R, S, T, V, W, or Y; wherein I403 is substituted with either an A, C, D, E, F, G, H, K, L, M, N, P, Q, R, S, T, V, W, or Y; wherein K404 is substituted with either an A, C, D, E, F, G, H, I, L, M, N, P, Q, R, S, T, V, W, or Y; wherein W405 is substituted with either an A, C, D, E, F, G, H, I, K, L, M, N, P, Q, R, S, T, V, or Y; wherein T406 is substituted with either an A, C, D, E, F, G, H, I, K, L, M, N, P, Q, R, S, V, W, or Y; wherein A407 is substituted with either a C, D, E, F, G, H, I, K, L, M, N, P, Q, R, S, T, V, W, or Y; wherein P408 is substituted with either an A, C, D, E, F, G, H, I, K, L, M, N, Q, R, S, T, V, W, or Y; wherein E409 is substituted with either an A, C, D, F, G, H, I, K, L, M, N, P, Q, R, S, T, V, W, or Y; wherein S410 is substituted with either an A, C, D, E, F, G, H, I, K, L, M, N, P, Q, R, T, V, W, or Y; wherein L411 is substituted with either an A, C, D, E, F, G, H, I, K, M, N, P, Q, R, S, T, V, W, or Y; wherein A412 is substituted with either a C, D, E, F, G, H, I, K, L, M, N, P, Q, R, S, T, V, W, or Y; wherein Y413 is substituted with either an A, C, D, E, F, G, H, I, K, L, M, N, P, Q, R, S, T, V, or W; wherein N414 is substituted with either an A, C, D, E, F, G, H, I, K, L, M, P, Q, R, S, T, V, W, or Y; wherein K415 is substituted with either an A, C, D, E, F, G, H, I, L, M, N, P, Q, R, S, T, V, W, or Y; wherein F416 is substituted with either an A, C, D, E, G, H, I, K, L, M, N, P, Q, R, S, T, V, W, or Y; wherein S417 is substituted with either an A, C, D, E, F, G, H, I, K, L, M, N, P, Q, R, T, V, W, or Y; wherein I418 is substituted with either an A, C, D, E, F, G, H, K, L, M, N, P, Q, R, S, T, V, W, or Y; wherein K419 is substituted with either an A, C, D, E, F, G, H, I, L, M, N, P, Q, R, S, T, V, W, or Y; wherein S420 is substituted with either an A, C, D, E, F, G, H, I, K, L, M, N, P, Q, R, T, V, W, or Y; wherein D421 is substituted with either an A, C, E, F, G, H, I, K, L, M, N, P, Q, R, S, T, V, W, or Y; wherein V422 is substituted with either an A, C, D, E, F, G, H, I, K, L, M, N, P, Q, R, S, T, W, or Y; wherein W423 is substituted with either an A, C, D, E, F, G, H, I, K, L, M, N, P, Q, R, S, T, V, or Y; wherein A424 is substituted with either a C, D, E, F, G, H, I, K, L, M, N, P, Q, R, S, T, V, W, or Y; wherein F425 is substituted with either an A, C, D, E, G, H, I, K, L, M, N, P, Q, R, S, T, V, W, or Y; wherein G426 is substituted with either an A, C, D, E, F, H, I, K, L, M, N, P, Q, R, S, T, V, W, or Y; wherein V427 is substituted with either an A, C, D, E, F, G, H, I, K, L, M, N, P, Q, R, S, T, W, or Y; wherein L428 is substituted with either an A, C, D, E, F, G, H, I, K, M, N, P, Q, R, S, T, V, W, or Y; wherein L429 is substituted with either an A, C, D, E, F, G, H, I, K, M, N, P, Q, R, S, T, V, W, or Y; wherein W430 is substituted with either an A, C, D, E, F, G, H, I, K, L, M, N, P, Q, R, S, T, V, or Y; wherein E431 is substituted with either an A, C, D, F, G, H, I, K, L, M, N, P, Q, R, S, T, V, W, or Y; wherein I432 is substituted with either an A, C, D, E, F, G, H, K, L, M, N, P, Q, R, S, T, V, W, or Y; wherein A433 is substituted with either a C, D, E, F, G, H, I, K, L, M, N, P, Q, R, S, T, V, W, or Y; wherein T434 is substituted with either an A, C, D, E, F, G, H, I, K, L, M, N, P, Q, R, S, V, W, or Y; wherein Y435 is substituted with either an A, C, D, E, F, G, H, I, K, L, M, N, P, Q, R, S, T, V, or W; wherein G436 is substituted with either an A, C, D, E, F, H, I, K, L, M, N, P, Q, R, S, T, V, W, or Y; wherein M437 is substituted with either an A, C, D, E, F, G, H, I, K, L, N, P, Q, R, S, T, V, W, or Y; wherein S438 is substituted with either an A, C, D, E, F, G, H, I, K, L, M, N, P, Q, R, T, V, W, or Y; wherein P439 is substituted with either an A, C, D, E, F, G, H, I, K, L, M, N, Q, R, S, T, V, W, or Y; wherein Y440 is substituted with either an A, C, D, E, F, G, H, I, K, L, M, N, P, Q, R, S, T, V, or W; wherein P441 is substituted with either an A, C, D, E, F, G, H, I, K, L, M, N, Q, R, S, T, V, W, or Y; wherein G442 is substituted with either an A, C, D, E, F, H, I, K, L, M, N, P, Q, R, S, T, V, W, or Y; wherein I443 is substituted with either an A, C, D, E, F, G, H, K, L, M, N, P, Q, R, S, T, V, W, or Y; wherein D444 is substituted with either an A, C, E, F, G, H, I, K, L, M, N, P, Q, R, S, T, V, W, or Y; wherein R445 is substituted with either an A, C, D, E, F, G, H, I, K, L, M, N, P, Q, S, T, V, W, or Y; wherein S446 is substituted with either an A, C, D, E, F, G, H, I, K, L, M, N, P, Q, R, T, V, W, or Y; wherein Q447 is substituted with either an A, C, D, E, F, G, H, I, K, L, M, N, P, R, S, T, V, W, or Y; wherein V448 is substituted with either an A, C, D, E, F, G, H, I, K, L, M, N, P, Q, R, S, T, W, or Y; wherein Y449 is substituted with either an A, C, D, E, F, G, H, I, K, L, M, N, P, Q, R, S, T, V, or W; wherein E450 is substituted with either an A, C, D, F, G, H, I, K, L, M, N, P, Q, R, S, T, V, W, or Y; wherein L451 is substituted with either an A, C, D, E, F, G, H, I, K, M, N, P, Q, R, S, T, V, W, or Y; wherein L452 is substituted with either an A, C, D, E, F, G, H, I, K, M, N, P, Q, R, S, T, V, W, or Y; wherein E453 is substituted with either an A, C, D, F, G, H, I, K, L, M, N, P, Q, R, S, T, V, W, or Y; wherein K454 is substituted with either an A, C, D, E, F, G, H, I, L, M, N, P, Q, R, S, T, V, W, or Y; wherein D455 is substituted with either an A, C, E, F, G, H, I, K, L, M, N, P, Q, R, S, T, V, W, or Y; wherein Y456 is substituted with either an A, C, D, E, F, G, H, I, K, L, M, N, P, Q, R, S, T, V, or W; wherein R457 is substituted with either an A, C, D, E, F, G, H, I, K, L, M, N, P, Q, S, T, V, W, or Y; wherein M458 is substituted with either an A, C, D, E, F, G, H, I, K, L, N, P, Q, R, S, T, V, W, or Y; wherein K459 is substituted with either an A, C, D, E, F, G, H, I, L, M, N, P, Q, R, S, T, V, W, or Y; wherein R460 is substituted with either an A, C, D, E, F, G, H, I, K, L, M, N, P, Q, S, T, V, W, or Y; wherein P461 is substituted with either an A, C, D, E, F, G, H, I, K, L, M, N, Q, R, S, T, V, W, or Y; wherein E462 is substituted with either an A, C, D, F, G, H, I, K, L, M, N, P, Q, R, S, T, V, W, or Y; wherein G463 is substituted with either an A, C, D, E, F, H, I, K, L, M, N, P, Q, R, S, T, V, W, or Y; wherein C464 is substituted with either an A, D, E, F, G, H, I, K, L, M, N, P, Q, R, S, T, V, W, or Y; wherein P465 is substituted with either an A, C, D, E, F, G, H, I, K, L, M, N, Q, R, S, T, V, W, or Y; wherein E466 is substituted with either an A, C, D, F, G, H, I, K, L, M, N, P, Q, R, S, T, V, W, or Y; wherein K467 is substituted with either an A, C, D, E, F, G, H, I, L, M, N, P, Q, R, S, T, V, W, or Y; wherein V468 is substituted with either an A, C, D, E, F, G, H, I, K, L, M, N, P, Q, R, S, T, W, or Y; wherein Y469 is substituted with either an A, C, D, E, F, G, H, I, K, L, M, N, P, Q, R, S, T, V, or W; wherein E470 is substituted with either an A, C, D, F, G, H, I, K, L, M, N, P, Q, R, S, T, V, W, or Y; wherein L471 is substituted with either an A, C, D, E, F, G, H, I, K, M, N, P, Q, R, S, T, V, W, or Y; wherein M472 is substituted with either an A, C, D, E, F, G, H, I, K, L, N, P, Q, R, S, T, V, W, or Y; wherein R473 is substituted with either an A, C, D, E, F, G, H, I, K, L, M, N, P, Q, S, T, V, W, or Y; wherein A474 is substituted with either a C, D, E, F, G, H, I, K, L, M, N, P, Q, R, S, T, V, W, or Y; wherein C475 is substituted with either an A, D, E, F, G, H, I, K, L, M, N, P, Q, R, S, T, V, W, or Y; wherein W476 is substituted with either an A, C, D, E, F, G, H, I, K, L, M, N, P, Q, R, S, T, V, or Y; wherein Q477 is substituted with either an A, C, D, E, F, G, H, I, K, L, M, N, P, R, S, T, V, W, or Y; wherein W478 is substituted with either an A, C, D, E, F, G, H, I, K, L, M, N, P, Q, R, S, T, V, or Y; wherein N479 is substituted with either an A, C, D, E, F, G, H, I, K, L, M, P, Q, R, S, T, V, W, or Y; wherein P480 is substituted with either an A, C, D, E, F, G, H, I, K, L, M, N, Q, R, S, T, V, W, or Y; wherein S481 is substituted with either an A, C, D, E, F, G, H, I, K, L, M, N, P, Q, R, T, V, W, or Y; wherein D482 is substituted with either an A, C, E, F, G, H, I, K, L, M, N, P, Q, R, S, T, V, W, or Y; wherein R483 is substituted with either an A, C, D, E, F, G, H, I, K, L, M, N, P, Q, S, T, V, W, or Y; wherein P484 is substituted with either an A, C, D, E, F, G, H, I, K, L, M, N, Q, R, S, T, V, W, or Y; wherein S485 is substituted with either an A, C, D, E, F, G, H, I, K, L, M, N, P, Q, R, T, V, W, or Y; wherein F486 is substituted with either an A, C, D, E, G, H, I, K, L, M, N, P, Q, R, S, T, V, W, or Y; wherein A487 is substituted with either a C, D, E, F, G, H, I, K, L, M, N, P, Q, R, S, T, V, W, or Y; wherein E488 is substituted with either an A, C, D, F, G, H, I, K, L, M, N, P, Q, R, S, T, V, W, or Y; wherein I489 is substituted with either an A, C, D, E, F, G, H, K, L, M, N, P, Q, R, S, T, V, W, or Y; wherein H490 is substituted with either an A, C, D, E, F, G, I, K, L, M, N, P, Q, R, S, T, V, W, or Y; wherein Q491 is substituted with either an A, C, D, E, F, G, H, I, K, L, M, N, P, R, S, T, V, W, or Y; wherein A492 is substituted with either a C, D, E, F, G, H, I, K, L, M, N, P, Q, R, S, T, V, W, or Y; wherein F493 is substituted with either an A, C, D, E, G, H, I, K, L, M, N, P, Q, R, S, T, V, W, or Y; wherein E494 is substituted with either an A, C, D, F, G, H, I, K, L, M, N, P, Q, R, S, T, V, W, or Y; wherein T495 is substituted with either an A, C, D, E, F, G, H, I, K, L, M, N, P, Q, R, S, V, W, or Y; wherein M496 is substituted with either an A, C, D, E, F, G, H, I, K, L, N, P, Q, R, S, T, V, W, or Y; wherein F497 is substituted with either an A, C, D, E, G, H, I, K, L, M, N, P, Q, R, S, T, V, W, or Y; wherein Q498 is substituted with either an A, C, D, E, F, G, H, I, K, L, M, N, P, R, S, T, V, W, or Y; wherein E499 is substituted with either an A, C, D, F, G, H, I, K, L, M, N, P, Q, R, S, T, V, W, or Y; wherein S500 is substituted with either an A, C, D, E, F, G, H, I, K, L, M, N, P, Q, R, T, V, W, or Y; wherein S501 is substituted with either an A, C, D, E, F, G, H, I, K, L, M, N, P, Q, R, T, V, W, or Y; wherein I502 is substituted with either an A, C, D, E, F, G, H, K, L, M, N, P, Q, R, S, T, V, W, or Y; wherein S503 is substituted with either an A, C, D, E, F, G, H, I, K, L, M, N, P, Q, R, T, V, W, or Y; wherein D504 is substituted with either an A, C, E, F, G, H, I, K, L, M, N, P, Q, R, S, T, V, W, or Y; wherein E505 is substituted with either an A, C, D, F, G, H, I, K, L, M, N, P, Q, R, S, T, V, W, or Y; wherein V506 is substituted with either an A, C, D, E, F, G, H, I, K, L, M, N, P, Q, R, S, T, W, or Y; wherein E507 is substituted with either an A, C, D, F, G, H, I, K, L, M, N, P, Q, R, S, T, V, W, or Y; wherein K508 is substituted with either an A, C, D, E, F, G, H, I, L, M, N, P, Q, R, S, T, V, W, or Y; wherein E509 is substituted with either an A, C, D, F, G, H, I, K, L, M, N, P, Q, R, S, T, V, W, or Y; wherein L510 is substituted with either an A, C, D, E, F, G, H, I, K, M, N, P, Q, R, S, T, V, W, or Y; wherein G511 is substituted with either an A, C, D, E, F, H, I, K, L, M, N, P, Q, R, S, T, V, W, or Y; wherein K512 is substituted with either an A, C, D, E, F, G, H, I, L, M, N, P, Q, R, S, T, V, W, or Y; wherein Q513 is substituted with either an A, C, D, E, F, G, H, I, K, L, M, N, P, R, S, T, V, W, or Y; wherein G514 is substituted with either an A, C, D, E, F, H, I, K, L, M, N, P, Q, R, S, T, V, W, or Y; wherein V515 is substituted with either an A, C, D, E, F, G, H, I, K, L, M, N, P, Q, R, S, T, W, or Y; wherein R516 is substituted with either an A, C, D, E, F, G, H, I, K, L, M, N, P, Q, S, T, V, W, or Y; wherein G517 is substituted with either an A, C, D, E, F, H, I, K, L, M, N, P, Q, R, S, T, V, W, or Y; wherein A518 is substituted with either a C, D, E, F, G, H, I, K, L, M, N, P, Q, R, S, T, V, W, or Y; wherein V519 is substituted with either an A, C, D, E, F, G, H, I, K, L, M, N, P, Q, R, S, T, W, or Y; wherein T520 is substituted with either an A, C, D, E, F, G, H, I, K, L, M, N, P, Q, R, S, V, W, or Y; wherein T521 is substituted with either an A, C, D, E, F, G, H, I, K, L, M, N, P, Q, R, S, V, W, or Y; wherein L522 is substituted with either an A, C, D, E, F, G, H, I, K, M, N, P, Q, R, S, T, V, W, or Y; wherein L523 is substituted with either an A, C, D, E, F, G, H, I, K, M, N, P, Q, R, S, T, V, W, or Y; wherein Q524 is substituted with either an A, C, D, E, F, G, H, I, K, L, M, N, P, R, S, T, V, W, or Y; wherein A525 is substituted with either a C, D, E, F, G, H, I, K, L, M, N, P, Q, R, S, T, V, W, or Y; wherein P526 is substituted with either an A, C, D, E, F, G, H, I, K, L, M, N, Q, R, S, T, V, W, or Y; wherein E527 is substituted with either an A, C, D, F, G, H, I, K, L, M, N, P, Q, R, S, T, V, W, or Y; wherein L528 is substituted with either an A, C, D, E, F, G, H, I, K, M, N, P, Q, R, S, T, V, W, or Y; wherein P529 is substituted with either an A, C, D, E, F, G, H, I, K, L, M, N, Q, R, S, T, V, W, or Y; wherein T530 is substituted with either an A, C, D, E, F, G, H, I, K, L, M, N, P, Q, R, S, V, W, or Y; wherein K531 is substituted with either an A, C, D, E, F, G, H, I, L, M, N, P, Q, R, S, T, V, W, or Y; wherein T532 is substituted with either an A, C, D, E, F, G, H, I, K, L, M, N, P, Q, R, S, V, W, or Y; wherein R533 is substituted with either an A, C, D, E, F, G, H, I, K, L, M, N, P, Q, S, T, V, W, or Y; wherein T534 is substituted with either an A, C, D, E, F, G, H, I, K, L, M, N, P, Q, R, S, V, W, or Y; wherein S535 is substituted with either an A, C, D, E, F, G, H, I, K, L, M, N, P, Q, R, T, V, W, or Y; wherein R536 is substituted with either an A, C, D, E, F, G, H, I, K, L, M, N, P, Q, S, T, V, W, or Y; wherein R537 is substituted with either an A, C, D, E, F, G, H, I, K, L, M, N, P, Q, S, T, V, W, or Y; wherein A538 is substituted with either a C, D, E, F, G, H, I, K, L, M, N, P, Q, R, S, T, V, W, or Y; wherein A539 is substituted with either a C, D, E, F, G, H, I, K, L, M, N, P, Q, R, S, T, V, W, or Y; wherein E540 is substituted with either an A, C, D, F, G, H, I, K, L, M, N, P, Q, R, S, T, V, W, or Y; wherein H541 is substituted with either an A, C, D, E, F, G, I, K, L, M, N, P, Q, R, S, T, V, W, or Y; wherein R542 is substituted with either an A, C, D, E, F, G, H, I, K, L, M, N, P, Q, S, T, V, W, or Y; wherein D543 is substituted with either an A, C, E, F, G, H, I, K, L, M, N, P, Q, R, S, T, V, W, or Y; wherein T544 is substituted with either an A, C, D, E, F, G, H, I, K, L, M, N, P, Q, R, S, V, W, or Y; wherein T545 is substituted with either an A, C, D, E, F, G, H, I, K, L, M, N, P, Q, R, S, V, W, or Y; wherein D546 is substituted with either an A, C, E, F, G, H, I, K, L, M, N, P, Q, R, S, T, V, W, or Y; wherein V547 is substituted with either an A, C, D, E, F, G, H, I, K, L, M, N, P, Q, R, S, T, W, or Y; wherein P548 is substituted with either an A, C, D, E, F, G, H, I, K, L, M, N, Q, R, S, T, V, W, or Y; wherein E549 is substituted with either an A, C, D, F, G, H, I, K, L, M, N, P, Q, R, S, T, V, W, or Y; wherein M550 is substituted with either an A, C, D, E, F, G, H, I, K, L, N, P, Q, R, S, T, V, W, or Y; wherein P551 is substituted with either an A, C, D, E, F, G, H, I, K, L, M, N, Q, R, S, T, V, W, or Y; wherein H552 is substituted with either an A, C, D, E, F, G, I, K, L, M, N, P, Q, R, S, T, V, W, or Y; wherein S553 is substituted with either an A, C, D, E, F, G, H, I, K, L, M, N, P, Q, R, T, V, W, or Y; wherein K554 is substituted with either an A, C, D, E, F, G, H, I, L, M, N, P, Q, R, S, T, V, W, or Y; wherein G555 is substituted with either an A, C, D, E, F, H, I, K, L, M, N, P, Q, R, S, T, V, W, or Y; wherein Q556 is substituted with either an A, C, D, E, F, G, H, I, K, L, M, N, P, R, S, T, V, W, or Y; wherein G557 is substituted with either an A, C, D, E, F, H, I, K, L, M, N, P, Q, R, S, T, V, W, or Y; wherein E558 is substituted with either an A, C, D, F, G, H, I, K, L, M, N, P, Q, R, S, T, V, W, or Y; wherein S559 is substituted with either an A, C, D, E, F, G, H, I, K, L, M, N, P, Q, R, T, V, W, or Y; wherein D560 is substituted with either an A, C, E, F, G, H, I, K, L, M, N, P, Q, R, S, T, V, W, or Y; wherein P561 is substituted with either an A, C, D, E, F, G, H, I, K, L, M, N, Q, R, S, T, V, W, or Y; wherein L562 is substituted with either an A, C, D, E, F, G, H, I, K, M, N, P, Q, R, S, T, V, W, or Y; wherein D563 is substituted with either an A, C, E, F, G, H, I, K, L, M, N, P, Q, R, S, T, V, W, or Y; wherein H564 is substituted with either an A, C, D, E, F, G, I, K, L, M, N, P, Q, R, S, T, V, W, or Y; wherein E565 is substituted with either an A, C, D, F, G, H, I, K, L, M, N, P, Q, R, S, T, V, W, or Y; wherein P566 is substituted with either an A, C, D, E, F, G, H, I, K, L, M, N, Q, R, S, T, V, W, or Y; wherein A567 is substituted with either a C, D, E, F, G, H, I, K, L, M, N, P, Q, R, S, T, V, W, or Y; wherein V568 is substituted with either an A, C, D, E, F, G, H, I, K, L, M, N, P, Q, R, S, T, W, or Y; wherein S569 is substituted with either an A, C, D, E, F, G, H, I, K, L, M, N, P, Q, R, T, V, W, or Y; wherein P570 is substituted with either an A, C, D, E, F, G, H, I, K, L, M, N, Q, R, S, T, V, W, or Y; wherein L571 is substituted with either an A, C, D, E, F, G, H, I, K, M, N, P, Q, R, S, T, V, W, or Y; wherein L572 is substituted with either an A, C, D, E, F, G, H, I, K, M, N, P, Q, R, S, T, V, W, or Y; wherein P573 is substituted with either an A, C, D, E, F, G, H, I, K, L, M, N, Q, R, S, T, V, W, or Y; wherein R574 is substituted with either an A, C, D, E, F, G, H, I, K, L, M, N, P, Q, S, T, V, W, or Y; wherein K575 is substituted with either an A, C, D, E, F, G, H, I, L, M, N, P, Q, R, S, T, V, W, or Y; wherein E576 is substituted with either an A, C, D, F, G, H, I, K, L, M, N, P, Q, R, S, T, V, W, or Y; wherein R577 is substituted with either an A, C, D, E, F, G, H, I, K, L, M, N, P, Q, S, T, V, W, or Y; wherein G578 is substituted with either an A, C, D, E, F, H, I, K, L, M, N, P, Q, R, S, T, V, W, or Y; wherein P579 is substituted with either an A, C, D, E, F, G, H, I, K, L, M, N, Q, R, S, T, V, W, or Y; wherein P580 is substituted with either an A, C, D, E, F, G, H, I, K, L, M, N, Q, R, S, T, V, W, or Y; wherein E581 is substituted with either an A, C, D, F, G, H, I, K, L, M, N, P, Q, R, S, T, V, W, or Y; wherein G582 is substituted with either an A, C, D, E, F, H, I, K, L, M, N, P, Q, R, S, T, V, W, or Y; wherein G583 is substituted with either an A, C, D, E, F, H, I, K, L, M, N, P, Q, R, S, T, V, W, or Y; wherein L584 is substituted with either an A, C, D, E, F, G, H, I, K, M, N, P, Q, R, S, T, V, W, or Y; wherein N585 is substituted with either an A, C, D, E, F, G, H, I, K, L, M, P, Q, R, S, T, V, W, or Y; wherein E586 is substituted with either an A, C, D, F, G, H, I, K, L, M, N, P, Q, R, S, T, V, W, or Y; wherein D587 is substituted with either an A, C, E, F, G, H, I, K, L, M, N, P, Q, R, S, T, V, W, or Y; wherein E588 is substituted with either an A, C, D, F, G, H, I, K, L, M, N, P, Q, R, S, T, V, W, or Y; wherein R589 is substituted with either an A, C, D, E, F, G, H, I, K, L, M, N, P, Q, S, T, V, W, or Y; wherein L590 is substituted with either an A, C, D, E, F, G, H, I, K, M, N, P, Q, R, S, T, V, W, or Y; wherein L591 is substituted with either an A, C, D, E, F, G, H, I, K, M, N, P, Q, R, S, T, V, W, or Y; wherein P592 is substituted with either an A, C, D, E, F, G, H, I, K, L, M, N, Q, R, S, T, V, W, or Y; wherein K593 is substituted with either an A, C, D, E, F, G, H, I, L, M, N, P, Q, R, S, T, V, W, or Y; wherein D594 is substituted with either an A, C, E, F, G, H, I, K, L, M, N, P, Q, R, S, T, V, W, or Y; wherein K595 is substituted with either an A, C, D, E, F, G, H, I, L, M, N, P, Q, R, S, T, V, W, or Y; wherein K596 is substituted with either an A, C, D, E, F, G, H, I, L, M, N, P, Q, R, S, T, V, W, or Y; wherein T597 is substituted with either an A, C, D, E, F, G, H, I, K, L, M, N, P, Q, R, S, V, W, or Y; wherein N598 is substituted with either an A, C, D, E, F, G, H, I, K, L, M, P, Q, R, S, T, V, W, or Y; wherein L599 is substituted with either an A, C, D, E, F, G, H, I, K, M, N, P, Q, R, S, T, V, W, or Y; wherein F600 is substituted with either an A, C, D, E, G, H, I, K, L, M, N, P, Q, R, S, T, V, W, or Y; wherein S601 is substituted with either an A, C, D, E, F, G, H, I, K, L, M, N, P, Q, R, T, V, W, or Y; wherein A602 is substituted with either a C, D, E, F, G, H, I, K, L, M, N, P, Q, R, S, T, V, W, or Y; wherein L603 is substituted with either an A, C, D, E, F, G, H, I, K, M, N, P, Q, R, S, T, V, W, or Y; wherein I604 is substituted with either an A, C, D, E, F, G, H, K, L, M, N, P, Q, R, S, T, V, W, or Y; wherein K605 is substituted with either an A, C, D, E, F, G, H, I, L, M, N, P, Q, R, S, T, V, W, or Y; wherein K606 is substituted with either an A, C, D, E, F, G, H, I, L, M, N, P, Q, R, S, T, V, W, or Y; wherein K607 is substituted with either an A, C, D, E, F, G, H, I, L, M, N, P, Q, R, S, T, V, W, or Y; wherein K608 is substituted with either an A, C, D, E, F, G, H, I, L, M, N, P, Q, R, S, T, V, W, or Y; wherein K609 is substituted with either an A, C, D, E, F, G, H, I, L, M, N, P, Q, R, S, T, V, W, or Y; wherein T610 is substituted with either an A, C, D, E, F, G, H, I, K, L, M, N, P, Q, R, S, V, W, or Y; wherein A611 is substituted with either a C, D, E, F, G, H, I, K, L, M, N, P, Q, R, S, T, V, W, or Y; wherein P612 is substituted with either an A, C, D, E, F, G, H, I, K, L, M, N, Q, R, S, T, V, W, or Y; wherein T613 is substituted with either an A, C, D, E, F, G, H, I, K, L, M, N, P, Q, R, S, V, W, or Y; wherein P614 is substituted with either an A, C, D, E, F, G, H, I, K, L, M, N, Q, R, S, T, V, W, or Y; wherein P615 is substituted with either an A, C, D, E, F, G, H, I, K, L, M, N, Q, R, S, T, V, W, or Y; wherein K616 is substituted with either an A, C, D, E, F, G, H, I, L, M, N, P, Q, R, S, T, V, W, or Y; wherein R617 is substituted with either an A, C, D, E, F, G, H, I, K, L, M, N, P, Q, S, T, V, W, or Y; wherein S618 is substituted with either an A, C, D, E, F, G, H, I, K, L, M, N, P, Q, R, T, V, W, or Y; wherein S619 is substituted with either an A, C, D, E, F, G, H, I, K, L, M, N, P, Q, R, T, V, W, or Y; wherein S620 is substituted with either an A, C, D, E, F, G, H, I, K, L, M, N, P, Q, R, T, V, W, or Y; wherein F621 is substituted with either an A, C, D, E, G, H, I, K, L, M, N, P, Q, R, S, T, V, W, or Y; wherein R622 is substituted with either an A, C, D, E, F, G, H, I, K, L, M, N, P, Q, S, T, V, W, or Y; wherein E623 is substituted with either an A, C, D, F, G, H, I, K, L, M, N, P, Q, R, S, T, V, W, or Y; wherein M624 is substituted with either an A, C, D, E, F, G, H, I, K, L, N, P, Q, R, S, T, V, W, or Y; wherein D625 is substituted with either an A, C, E, F, G, H, I, K, L, M, N, P, Q, R, S, T, V, W, or Y; wherein G626 is substituted with either an A, C, D, E, F, H, I, K, L, M, N, P, Q, R, S, T, V, W, or Y; wherein Q627 is substituted with either an A, C, D, E, F, G, H, I, K, L, M, N, P, R, S, T, V, W, or Y; wherein P628 is substituted with either an A, C, D, E, F, G, H, I, K, L, M, N, Q, R, S, T, V, W, or Y; wherein E629 is substituted with either an A, C, D, F, G, H, I, K, L, M, N, P, Q, R, S, T, V, W, or Y; wherein R630 is substituted with either an A, C, D, E, F, G, H, I, K, L, M, N, P, Q, S, T, V, W, or Y; wherein R631 is substituted with either an A, C, D, E, F, G, H, I, K, L, M, N, P, Q, S, T, V, W, or Y; wherein G632 is substituted with either an A, C, D, E, F, H, I, K, L, M, N, P, Q, R, S, T, V, W, or Y; wherein A633 is substituted with either a C, D, E, F, G, H, I, K, L, M, N, P, Q, R, S, T, V, W, or Y; wherein G634 is substituted with either an A, C, D, E, F, H, I, K, L, M, N, P, Q, R, S, T, V, W, or Y; wherein E635 is substituted with either an A, C, D, F, G, H, I, K, L, M, N, P, Q, R, S, T, V, W, or Y; wherein E636 is substituted with either an A, C, D, F, G, H, I, K, L, M, N, P, Q, R, S, T, V, W, or Y; wherein E637 is substituted with either an A, C, D, F, G, H, I, K, L, M, N, P, Q, R, S, T, V, W, or Y; wherein G638 is substituted with either an A, C, D, E, F, H, I, K, L, M, N, P, Q, R, S, T, V, W, or Y; wherein R639 is substituted with either an A, C, D, E, F, G, H, I, K, L, M, N, P, Q, S, T, V, W, or Y; wherein D640 is substituted with either an A, C, E, F, G, H, I, K, L, M, N, P, Q, R, S, T, V, W, or Y; wherein I641 is substituted with either an A, C, D, E, F, G, H, K, L, M, N, P, Q, R, S, T, V, W, or Y; wherein S642 is substituted with either an A, C, D, E, F, G, H, I, K, L, M, N, P, Q, R, T, V, W, or Y; wherein N643 is substituted with either an A, C, D, E, F, G, H, I, K, L, M, P, Q, R, S, T, V, W, or Y; wherein G644 is substituted with either an A, C, D, E, F, H, I, K, L, M, N, P, Q, R, S, T, V, W, or Y; wherein A645 is substituted with either a C, D, E, F, G, H, I, K, L, M, N, P, Q, R, S, T, V, W, or Y; wherein L646 is substituted with either an A, C, D, E, F, G, H, I, K, M, N, P, Q, R, S, T, V, W, or Y; wherein A647 is substituted with either a C, D, E, F, G, H, I, K, L, M, N, P, Q, R, S, T, V, W, or Y; wherein F648 is substituted with either an A, C, D, E, G, H, I, K, L, M, N, P, Q, R, S, T, V, W, or Y; wherein T649 is substituted with either an A, C, D, E, F, G, H, I, K, L, M, N, P, Q, R, S, V, W, or Y; wherein P650 is substituted with either an A, C, D, E, F, G, H, I, K, L, M, N, Q, R, S, T, V, W, or Y; wherein L651 is substituted with either an A, C, D, E, F, G, H, I, K, M, N, P, Q, R, S, T, V, W, or Y; wherein D652 is substituted with either an A, C, E, F, G, H, I, K, L, M, N, P, Q, R, S, T, V, W, or Y; wherein T653 is substituted with either an A, C, D, E, F, G, H, I, K, L, M, N, P, Q, R, S, V, W, or Y; wherein A654 is substituted with either a C, D, E, F, G, H, I, K, L, M, N, P, Q, R, S, T, V, W, or Y; wherein D655 is substituted with either an A, C, E, F, G, H, I, K, L, M, N, P, Q, R, S, T, V, W, or Y; wherein P656 is substituted with either an A, C, D, E, F, G, H, I, K, L, M, N, Q, R, S, T, V, W, or Y; wherein A657 is substituted with either a C, D, E, F, G, H, I, K, L, M, N, P, Q, R, S, T, V, W, or Y; wherein K658 is substituted with either an A, C, D, E, F, G, H, I, L, M, N, P, Q, R, S, T, V, W, or Y; wherein S659 is substituted with either an A, C, D, E, F, G, H, I, K, L, M, N, P, Q, R, T, V, W, or Y; wherein P660 is substituted with either an A, C, D, E, F, G, H, I, K, L, M, N, Q, R, S, T, V, W, or Y; wherein K661 is substituted with either an A, C, D, E, F, G, H, I, L, M, N, P, Q, R, S, T, V, W, or Y; wherein P662 is substituted with either an A, C, D, E, F, G, H, I, K, L, M, N, Q, R, S, T, V, W, or Y; wherein S663 is substituted with either an A, C, D, E, F, G, H, I, K, L, M, N, P, Q, R, T, V, W, or Y; wherein N664 is substituted with either an A, C, D, E, F, G, H, I, K, L, M, P, Q, R, S, T, V, W, or Y; wherein G665 is substituted with either an A, C, D, E, F, H, I, K, L, M, N, P, Q, R, S, T, V, W, or Y; wherein A666 is substituted with either a C, D, E, F, G, H, I, K, L, M, N, P, Q, R, S, T, V, W, or Y; wherein G667 is substituted with either an A, C, D, E, F, H, I, K, L, M, N, P, Q, R, S, T, V, W, or Y; wherein V668 is substituted with either an A, C, D, E, F, G, H, I, K, L, M, N, P, Q, R, S, T, W, or Y; wherein P669 is substituted with either an A, C, D, E, F, G, H, I, K, L, M, N, Q, R, S, T, V, W, or Y; wherein N670 is substituted with either an A, C, D, E, F, G, H, I, K, L, M, P, Q, R, S, T, V, W, or Y; wherein G671 is substituted with either an A, C, D, E, F, H, I, K, L, M, N, P, Q, R, S, T, V, W, or Y; wherein A672 is substituted with either a C, D, E, F, G, H, I, K, L, M, N, P, Q, R, S, T, V, W, or Y; wherein L673 is substituted with either an A, C, D, E, F, G, H, I, K, M, N, P, Q, R, S, T, V, W, or Y; wherein R674 is substituted with either an A, C, D, E, F, G, H, I, K, L, M, N, P, Q, S, T, V, W, or Y; wherein E675 is substituted with either an A, C, D, F, G, H, I, K, L, M, N, P, Q, R, S, T, V, W, or Y; wherein S676 is substituted with either an A, C, D, E, F, G, H, I, K, L, M, N, P, Q, R, T, V, W, or Y; wherein G677 is substituted with either an A, C, D, E, F, H, I, K, L, M, N, P, Q, R, S, T, V, W, or Y; wherein G678 is substituted with either an A, C, D, E, F, H, I, K, L, M, N, P, Q, R, S, T, V, W, or Y; wherein S679 is substituted with either an A, C, D, E, F, G, H, I, K, L, M, N, P, Q, R, T, V, W, or Y; wherein G680 is substituted with either an A, C, D, E, F, H, I, K, L, M, N, P, Q, R, S, T, V, W, or Y; wherein F681 is substituted with either an A, C, D, E, G, H, I, K, L, M, N, P, Q, R, S, T, V, W, or Y; wherein R682 is substituted with either an A, C, D, E, F, G, H, I, K, L, M, N, P, Q, S, T, V, W, or Y; wherein S683 is substituted with either an A, C, D, E, F, G, H, I, K, L, M, N, P, Q, R, T, V, W, or Y; wherein P684 is substituted with either an A, C, D, E, F, G, H, I, K, L, M, N, Q, R, S, T, V, W, or Y; wherein H685 is substituted with either an A, C, D, E, F, G, I, K, L, M, N, P, Q, R, S, T, V, W, or Y; wherein L686 is substituted with either an A, C, D, E, F, G, H, I, K, M, N, P, Q, R, S, T, V, W, or Y; wherein W687 is substituted with either an A, C, D, E, F, G, H, I, K, L, M, N, P, Q, R, S, T, V, or Y; wherein K688 is substituted with either an A, C, D, E, F, G, H, I, L, M, N, P, Q, R, S, T, V, W, or Y; wherein K689 is substituted with either an A, C, D, E, F, G, H, I, L, M, N, P, Q, R, S, T, V, W, or Y; wherein S690 is substituted with either an A, C, D, E, F, G, H, I, K, L, M, N, P, Q, R, T, V, W, or Y; wherein S691 is substituted with either an A, C, D, E, F, G, H, I, K, L, M, N, P, Q, R, T, V, W, or Y; wherein T692 is substituted with either an A, C, D, E, F, G, H, I, K, L, M, N, P, Q, R, S, V, W, or Y; wherein L693 is substituted with either an A, C, D, E, F, G, H, I, K, M, N, P, Q, R, S, T, V, W, or Y; wherein T694 is substituted with either an A, C, D, E, F, G, H, I, K, L, M, N, P, Q, R, S, V, W, or Y; wherein S695 is substituted with either an A, C, D, E, F, G, H, I, K, L, M, N, P, Q, R, T, V, W, or Y; wherein S696 is substituted with either an A, C, D, E, F, G, H, I, K, L, M, N, P, Q, R, T, V, W, or Y; wherein R697 is substituted with either an A, C, D, E, F, G, H, I, K, L, M, N, P, Q, S, T, V, W, or Y; wherein L698 is substituted with either an A, C, D, E, F, G, H, I, K, M, N, P, Q, R, S, T, V, W, or Y; wherein A699 is substituted with either a C, D, E, F, G, H, I, K, L, M, N, P, Q, R, S, T, V, W, or Y; wherein T700 is substituted with either an A, C, D, E, F, G, H, I, K, L, M, N, P, Q, R, S, V, W, or Y; wherein G701 is substituted with either an A, C, D, E, F, H, I, K, L, M, N, P, Q, R, S, T, V, W, or Y; wherein E702 is substituted with either an A, C, D, F, G, H, I, K, L, M, N, P, Q, R, S, T, V, W, or Y; wherein E703 is substituted with either an A, C, D, F, G, H, I, K, L, M, N, P, Q, R, S, T, V, W, or Y; wherein E704 is substituted with either an A, C, D, F, G, H, I, K, L, M, N, P, Q, R, S, T, V, W, or Y; wherein G705 is substituted with either an A, C, D, E, F, H, I, K, L, M, N, P, Q, R, S, T, V, W, or Y; wherein G706 is substituted with either an A, C, D, E, F, H, I, K, L, M, N, P, Q, R, S, T, V, W, or Y; wherein G707 is substituted with either an A, C, D, E, F, H, I, K, L, M, N, P, Q, R, S, T, V, W, or Y; wherein S708 is substituted with either an A, C, D, E, F, G, H, I, K, L, M, N, P, Q, R, T, V, W, or Y; wherein S709 is substituted with either an A, C, D, E, F, G, H, I, K, L, M, N, P, Q, R, T, V, W, or Y; wherein S710 is substituted with either an A, C, D, E, F, G, H, I, K, L, M, N, P, Q, R, T, V, W, or Y; wherein K711 is substituted with either an A, C, D, E, F, G, H, I, L, M, N, P, Q, R, S, T, V, W, or Y; wherein R712 is substituted with either an A, C, D, E, F, G, H, I, K, L, M, N, P, Q, S, T, V, W, or Y; wherein F713 is substituted with either an A, C, D, E, G, H, I, K, L, M, N, P, Q, R, S, T, V, W, or Y; wherein L714 is substituted with either an A, C, D, E, F, G, H, I, K, M, N, P, Q, R, S, T, V, W, or Y; wherein R715 is substituted with either an A, C, D, E, F, G, H, I, K, L, M, N, P, Q, S, T, V, W, or Y; wherein S716 is substituted with either an A, C, D, E, F, G, H, I, K, L, M, N, P, Q, R, T, V, W, or Y; wherein C717 is substituted with either an A, D, E, F, G, H, I, K, L, M, N, P, Q, R, S, T, V, W, or Y; wherein S718 is substituted with either an A, C, D, E, F, G, H, I, K, L, M, N, P, Q, R, T, V, W, or Y; wherein V719 is substituted with either an A, C, D, E, F, G, H, I, K, L, M, N, P, Q, R, S, T, W, or Y; wherein S720 is substituted with either an A, C, D, E, F, G, H, I, K, L, M, N, P, Q, R, T, V, W, or Y; wherein C721 is substituted with either an A, D, E, F, G, H, I, K, L, M, N, P, Q, R, S, T, V, W, or Y; wherein V722 is substituted with either an A, C, D, E, F, G, H, I, K, L, M, N, P, Q, R, S, T, W, or Y; wherein P723 is substituted with either an A, C, D, E, F, G, H, I, K, L, M, N, Q, R, S, T, V, W, or Y; wherein H724 is substituted with either an A, C, D, E, F, G, I, K, L, M, N, P, Q, R, S, T, V, W, or Y; wherein G725 is substituted with either an A, C, D, E, F, H, I, K, L, M, N, P, Q, R, S, T, V, W, or Y; wherein A726 is substituted with either a C, D, E, F, G, H, I, K, L, M, N, P, Q, R, S, T, V, W, or Y; wherein K727 is substituted with either an A, C, D, E, F, G, H, I, L, M, N, P, Q, R, S, T, V, W, or Y; wherein D728 is substituted with either an A, C, E, F, G, H, I, K, L, M, N, P, Q, R, S, T, V, W, or Y; wherein T729 is substituted with either an A, C, D, E, F, G, H, I, K, L, M, N, P, Q, R, S, V, W, or Y; wherein E730 is substituted with either an A, C, D, F, G, H, I, K, L, M, N, P, Q, R, S, T, V, W, or Y; wherein W731 is substituted with either an A, C, D, E, F, G, H, I, K, L, M, N, P, Q, R, S, T, V, or Y; wherein R732 is substituted with either an A, C, D, E, F, G, H, I, K, L, M, N, P, Q, S, T, V, W, or Y; wherein S733 is substituted with either an A, C, D, E, F, G, H, I, K, L, M, N, P, Q, R, T, V, W, or Y; wherein V734 is substituted with either an A, C, D, E, F, G, H, I, K, L, M, N, P, Q, R, S, T, W, or Y; wherein T735 is substituted with either an A, C, D, E, F, G, H, I, K, L, M, N, P, Q, R, S, V, W, or Y; wherein L736 is substituted with either an A, C, D, E, F, G, H, I, K, M, N, P, Q, R, S, T, V, W, or Y; wherein P737 is substituted with either an A, C, D, E, F, G, H, I, K, L, M, N, Q, R, S, T, V, W, or Y; wherein R738 is substituted with either an A, C, D, E, F, G, H, I, K, L, M, N, P, Q, S, T, V, W, or Y; wherein D739 is substituted with either an A, C, E, F, G, H, I, K, L, M, N, P, Q, R, S, T, V, W, or Y; wherein L740 is substituted with either an A, C, D, E, F, G, H, I, K, M, N, P, Q, R, S, T, V, W, or Y; wherein Q741 is substituted with either an A, C, D, E, F, G, H, I, K, L, M, N, P, R, S, T, V, W, or Y; wherein S742 is substituted with either an A, C, D, E, F, G, H, I, K, L, M, N, P, Q, R, T, V, W, or Y; wherein T743 is substituted with either an A, C, D, E, F, G, H, I, K, L, M, N, P, Q, R, S, V, W, or Y; wherein G744 is substituted with either an A, C, D, E, F, H, I, K, L, M, N, P, Q, R, S, T, V, W, or Y; wherein R745 is substituted with either an A, C, D, E, F, G, H, I, K, L, M, N, P, Q, S, T, V, W, or Y; wherein Q746 is substituted with either an A, C, D, E, F, G, H, I, K, L, M, N, P, R, S, T, V, W, or Y; wherein F747 is substituted with either an A, C, D, E, G, H, I, K, L, M, N, P, Q, R, S, T, V, W, or Y; wherein D748 is substituted with either an A, C, E, F, G, H, I, K, L, M, N, P, Q, R, S, T, V, W, or Y; wherein S749 is substituted with either an A, C, D, E, F, G, H, I, K, L, M, N, P, Q, R, T, V, W, or Y; wherein S750 is substituted with either an A, C, D, E, F, G, H, I, K, L, M, N, P, Q, R, T, V, W, or Y; wherein T751 is substituted with either an A, C, D, E, F, G, H, I, K, L, M, N, P, Q, R, S, V, W, or Y; wherein F752 is substituted with either an A, C, D, E, G, H, I, K, L, M, N, P, Q, R, S, T, V, W, or Y; wherein G753 is substituted with either an A, C, D, E, F, H, I, K, L, M, N, P, Q, R, S, T, V, W, or Y; wherein G754 is substituted with either an A, C, D, E, F, H, I, K, L, M, N, P, Q, R, S, T, V, W, or Y; wherein H755 is substituted with either an A, C, D, E, F, G, I, K, L, M, N, P, Q, R, S, T, V, W, or Y; wherein K756 is substituted with either an A, C, D, E, F, G, H, I, L, M, N, P, Q, R, S, T, V, W, or Y; wherein S757 is substituted with either an A, C, D, E, F, G, H, I, K, L, M, N, P, Q, R, T, V, W, or Y; wherein E758 is substituted with either an A, C, D, F, G, H, I, K, L, M, N, P, Q, R, S, T, V, W, or Y; wherein K759 is substituted with either an A, C, D, E, F, G, H, I, L, M, N, P, Q, R, S, T, V, W, or Y; wherein P760 is substituted with either an A, C, D, E, F, G, H, I, K, L, M, N, Q, R, S, T, V, W, or Y; wherein A761 is substituted with either a C, D, E, F, G, H, I, K, L, M, N, P, Q, R, S, T, V, W, or Y; wherein L762 is substituted with either an A, C, D, E, F, G, H, I, K, M, N, P, Q, R, S, T, V, W, or Y; wherein P763 is substituted with either an A, C, D, E, F, G, H, I, K, L, M, N, Q, R, S, T, V, W, or Y; wherein R764 is substituted with either an A, C, D, E, F, G, H, I, K, L, M, N, P, Q, S, T, V, W, or Y; wherein K765 is substituted with either an A, C, D, E, F, G, H, I, L, M, N, P, Q, R, S, T, V, W, or Y; wherein R766 is substituted with either an A, C, D, E, F, G, H, I, K, L, M, N, P, Q, S, T, V, W, or Y; wherein A767 is substituted with either a C, D, E, F, G, H, I, K, L, M, N, P, Q, R, S, T, V, W, or Y; wherein G768 is substituted with either an A, C, D, E, F, H, I, K, L, M, N, P, Q, R, S, T, V, W, or Y; wherein E769 is substituted with either an A, C, D, F, G, H, I, K, L, M, N, P, Q, R, S, T, V, W, or Y; wherein N770 is substituted with either an A, C, D, E, F, G, H, I, K, L, M, P, Q, R, S, T, V, W, or Y; wherein R771 is substituted with either an A, C, D, E, F, G, H, I, K, L, M, N, P, Q, S, T, V, W, or Y; wherein S772 is substituted with either an A, C, D, E, F, G, H, I, K, L, M, N, P, Q, R, T, V, W, or Y; wherein D773 is substituted with either an A, C, E, F, G, H, I, K, L, M, N, P, Q, R, S, T, V, W, or Y; wherein Q774 is substituted with either an A, C, D, E, F, G, H, I, K, L, M, N, P, R, S, T, V, W, or Y; wherein V775 is substituted with either an A, C, D, E, F, G, H, I, K, L, M, N, P, Q, R, S, T, W, or Y; wherein T776 is substituted with either an A, C, D, E, F, G, H, I, K, L, M, N, P, Q, R, S, V, W, or Y; wherein R777 is substituted with either an A, C, D, E, F, G, H, I, K, L, M, N, P, Q, S, T, V, W, or Y; wherein G778 is substituted with either an A, C, D, E, F, H, I, K, L, M, N, P, Q, R, S, T, V, W, or Y; wherein T779 is substituted with either an A, C, D, E, F, G, H, I, K, L, M, N, P, Q, R, S, V, W, or Y; wherein V780 is substituted with either an A, C, D, E, F, G, H, I, K, L, M, N, P, Q, R, S, T, W, or Y; wherein T781 is substituted with either an A, C, D, E, F, G, H, I, K, L, M, N, P, Q, R, S, V, W, or Y; wherein P782 is substituted with either an A, C, D, E, F, G, H, I, K, L, M, N, Q, R, S, T, V, W, or Y; wherein P783 is substituted with either an A, C, D, E, F, G, H, I, K, L, M, N, Q, R, S, T, V, W, or Y; wherein P784 is substituted with either an A, C, D, E, F, G, H, I, K, L, M, N, Q, R, S, T, V, W, or Y; wherein R785 is substituted with either an A, C, D, E, F, G, H, I, K, L, M, N, P, Q, S, T, V, W, or Y; wherein L786 is substituted with either an A, C, D, E, F, G, H, I, K, M, N, P, Q, R, S, T, V, W, or Y; wherein V787 is substituted with either an A, C, D, E, F, G, H, I, K, L, M, N, P, Q, R, S, T, W, or Y; wherein K788 is substituted with either an A, C, D, E, F, G, H, I, L, M, N, P, Q, R, S, T, V, W, or Y; wherein K789 is substituted with either an A, C, D, E, F, G, H, I, L, M, N, P, Q, R, S, T, V, W, or Y; wherein N790 is substituted with either an A, C, D, E, F, G, H, I, K, L, M, P, Q, R, S, T, V, W, or Y; wherein E791 is substituted with either an A, C, D, F, G, H, I, K, L, M, N, P, Q, R, S, T, V, W, or Y; wherein E792 is substituted with either an A, C, D, F, G, H, I, K, L, M, N, P, Q, R, S, T, V, W, or Y; wherein A793 is substituted with either a C, D, E, F, G, H, I, K, L, M, N, P, Q, R, S, T, V, W, or Y; wherein A794 is substituted with either a C, D, E, F, G, H, I, K, L, M, N, P, Q, R, S, T, V, W, or Y; wherein D795 is substituted with either an A, C, E, F, G, H, I, K, L, M, N, P, Q, R, S, T, V, W, or Y; wherein E796 is substituted with either an A, C, D, F, G, H, I, K, L, M, N, P, Q, R, S, T, V, W, or Y; wherein V797 is substituted with either an A, C, D, E, F, G, H, I, K, L, M, N, P, Q, R, S, T, W, or Y; wherein F798 is substituted with either an A, C, D, E, G, H, I, K, L, M, N, P, Q, R, S, T, V, W, or Y; wherein K799 is substituted with either an A, C, D, E, F, G, H, I, L, M, N, P, Q, R, S, T, V, W, or Y; wherein D800 is substituted with either an A, C, E, F, G, H, I, K, L, M, N, P, Q, R, S, T, V, W, or Y; wherein I801 is substituted with either an A, C, D, E, F, G, H, K, L, M, N, P, Q, R, S, T, V, W, or Y; wherein M802 is substituted with either an A, C, D, E, F, G, H, I, K, L, N, P, Q, R, S, T, V, W, or Y; wherein E803 is substituted with either an A, C, D, F, G, H, I, K, L, M, N, P, Q, R, S, T, V, W, or Y; wherein S804 is substituted with either an A, C, D, E, F, G, H, I, K, L, M, N, P, Q, R, T, V, W, or Y; wherein S805 is substituted with either an A, C, D, E, F, G, H, I, K, L, M, N, P, Q, R, T, V, W, or Y; wherein P806 is substituted with either an A, C, D, E, F, G, H, I, K, L, M, N, Q, R, S, T, V, W, or Y; wherein G807 is substituted with either an A, C, D, E, F, H, I, K, L, M, N, P, Q, R, S, T, V, W, or Y; wherein S808 is substituted with either an A, C, D, E, F, G, H, I, K, L, M, N, P, Q, R, T, V, W, or Y; wherein S809 is substituted with either an A, C, D, E, F, G, H, I, K, L, M, N, P, Q, R, T, V, W, or Y; wherein P810 is substituted with either an A, C, D, E, F, G, H, I, K, L, M, N, Q, R, S, T, V, W, or Y; wherein P811 is substituted with either an A, C, D, E, F, G, H, I, K, L, M, N, Q, R, S, T, V, W, or Y; wherein N812 is substituted with either an A, C, D, E, F, G, H, I, K, L, M, P, Q, R, S, T, V, W, or Y; wherein L813 is substituted with either an A, C, D, E, F, G, H, I, K, M, N, P, Q, R, S, T, V, W, or Y; wherein T814 is substituted with either an A, C, D, E, F, G, H, I, K, L, M, N, P, Q, R, S, V, W, or Y; wherein P815 is substituted with either an A, C, D, E, F, G, H, I, K, L, M, N, Q, R, S, T, V, W, or Y; wherein K816 is substituted with either an A, C, D, E, F, G, H, I, L, M, N, P, Q, R, S, T, V, W, or Y; wherein P817 is substituted with either an A, C, D, E, F, G, H, I, K, L, M, N, Q, R, S, T, V, W, or Y; wherein L818 is substituted with either an A, C, D, E, F, G, H, I, K, M, N, P, Q, R, S, T, V, W, or Y; wherein R819 is substituted with either an A, C, D, E, F, G, H, I, K, L, M, N, P, Q, S, T, V, W, or Y; wherein R820 is substituted with either an A, C, D, E, F, G, H, I, K, L, M, N, P, Q, S, T, V, W, or Y; wherein Q821 is substituted with either an A, C, D, E, F, G, H, I, K, L, M, N, P, R, S, T, V, W, or Y; wherein V822 is substituted with either an A, C, D, E, F, G, H, I, K, L, M, N, P, Q, R, S, T, W, or Y; wherein T823 is substituted with either an A, C, D, E, F, G, H, I, K, L, M, N, P, Q, R, S, V, W, or Y; wherein V824 is substituted with either an A, C, D, E, F, G, H, I, K, L, M, N, P, Q, R, S, T, W, or Y; wherein A825 is substituted with either a C, D, E, F, G, H, I, K, L, M, N, P, Q, R, S, T, V, W, or Y; wherein P826 is substituted with either an A, C, D, E, F, G, H, I, K, L, M, N, Q, R, S, T, V, W, or Y; wherein A827 is substituted with either a C, D, E, F, G, H, I, K, L, M, N, P, Q, R, S, T, V, W, or Y; wherein S828 is substituted with either an A, C, D, E, F, G, H, I, K, L, M, N, P, Q, R, T, V, W, or Y; wherein G829 is substituted with either an A, C, D, E, F, H, I, K, L, M, N, P, Q, R, S, T, V, W, or Y; wherein L830 is substituted with either an A, C, D, E, F, G, H, I, K, M, N, P, Q, R, S, T, V, W, or Y; wherein P831 is substituted with either an A, C, D, E, F, G, H, I, K, L, M, N, Q, R, S, T, V, W, or Y; wherein H832 is substituted with either an A, C, D, E, F, G, I, K, L, M, N, P, Q, R, S, T, V, W, or Y; wherein K833 is substituted with either an A, C, D, E, F, G, H, I, L, M, N, P, Q, R, S, T, V, W, or Y; wherein E834 is substituted with either an A, C, D, F, G, H, I, K, L, M, N, P, Q, R, S, T, V, W, or Y; wherein E835 is substituted with either an A, C, D, F, G, H, I, K, L, M, N, P, Q, R, S, T, V, W, or Y; wherein A836 is substituted with either a C, D, E, F, G, H, I, K, L, M, N, P, Q, R, S, T, V, W, or Y; wherein W837 is substituted with either an A, C, D, E, F, G, H, I, K, L, M, N, P, Q, R, S, T, V, or Y; wherein K838 is substituted with either an A, C, D, E, F, G, H, I, L, M, N, P, Q, R, S, T, V, W, or Y; wherein G839 is substituted with either an A, C, D, E, F, H, I, K, L, M, N, P, Q, R, S, T, V, W, or Y; wherein S840 is substituted with either an A, C, D, E, F, G, H, I, K, L, M, N, P, Q, R, T, V, W, or Y; wherein A841 is substituted with either a C, D, E, F, G, H, I, K, L, M, N, P, Q, R, S, T, V, W, or Y; wherein L842 is substituted with either an A, C, D, E, F, G, H, I, K, M, N, P, Q, R, S, T, V, W, or Y; wherein G843 is substituted with either an A, C, D, E, F, H, I, K, L, M, N, P, Q, R, S, T, V, W, or Y; wherein T844 is substituted with either an A, C, D, E, F, G, H, I, K, L, M, N, P, Q, R, S, V, W, or Y; wherein P845 is substituted with either an A, C, D, E, F, G, H, I, K, L, M, N, Q, R, S, T, V, W, or Y; wherein A846 is substituted with either a C, D, E, F, G, H, I, K, L, M, N, P, Q, R, S, T, V, W, or Y; wherein A847 is substituted with either a C, D, E, F, G, H, I, K, L, M, N, P, Q, R, S, T, V, W, or Y; wherein A848 is substituted with either a C, D, E, F, G, H, I, K, L, M, N, P, Q, R, S, T, V, W, or Y; wherein E849 is substituted with either an A, C, D, F, G, H, I, K, L, M, N, P, Q, R, S, T, V, W, or Y; wherein P850 is substituted with either an A, C, D, E, F, G, H, I, K, L, M, N, Q, R, S, T, V, W, or Y; wherein V851 is substituted with either an A, C, D, E, F, G, H, I, K, L, M, N, P, Q, R, S, T, W, or Y; wherein T852 is substituted with either an A, C, D, E, F, G, H, I, K, L, M, N, P, Q, R, S, V, W, or Y; wherein P853 is substituted with either an A, C, D, E, F, G, H, I, K, L, M, N, Q, R, S, T, V, W, or Y; wherein T854 is substituted with either an A, C, D, E, F, G, H, I, K, L, M, N, P, Q, R, S, V, W, or Y; wherein S855 is substituted with either an A, C, D, E, F, G, H, I, K, L, M, N, P, Q, R, T, V, W, or Y; wherein K856 is substituted with either an A, C, D, E, F, G, H, I, L, M, N, P, Q, R, S, T, V, W, or Y; wherein A857 is substituted with either a C, D, E, F, G, H, I, K, L, M, N, P, Q, R, S, T, V, W, or Y; wherein G858 is substituted with either an A, C, D, E, F, H, I, K, L, M, N, P, Q, R, S, T, V, W, or Y; wherein S859 is substituted with either an A, C, D, E, F, G, H, I, K, L, M, N, P, Q, R, T, V, W, or Y; wherein G860 is substituted with either an A, C, D, E, F, H, I, K, L, M, N, P, Q, R, S, T, V, W, or Y; wherein A861 is substituted with either a C, D, E, F, G, H, I, K, L, M, N, P, Q, R, S, T, V, W, or Y; wherein P862 is substituted with either an A, C, D, E, F, G, H, I, K, L, M, N, Q, R, S, T, V, W, or Y; wherein R863 is substituted with either an A, C, D, E, F, G, H, I, K, L, M, N, P, Q, S, T, V, W, or Y; wherein G864 is substituted with either an A, C, D, E, F, H, I, K, L, M, N, P, Q, R, S, T, V, W, or Y; wherein T865 is substituted with either an A, C, D, E, F, G, H, I, K, L, M, N, P, Q, R, S, V, W, or Y; wherein S866 is substituted with either an A, C, D, E, F, G, H, I, K, L, M, N, P, Q, R, T, V, W, or Y; wherein K867 is substituted with either an A, C, D, E, F, G, H, I, L, M, N, P, Q, R, S, T, V, W, or Y; wherein G868 is substituted with either an A, C, D, E, F, H, I, K, L, M, N, P, Q, R, S, T, V, W, or Y; wherein P869 is substituted with either an A, C, D, E, F, G, H, I, K, L, M, N, Q, R, S, T, V, W, or Y; wherein A870 is substituted with either a C, D, E, F, G, H, I, K, L, M, N, P, Q, R, S, T, V, W, or Y; wherein E871 is substituted with either an A, C, D, F, G, H, I, K, L, M, N, P, Q, R, S, T, V, W, or Y; wherein E872 is substituted with either an A, C, D, F, G, H, I, K, L, M, N, P, Q, R, S, T, V, W, or Y; wherein S873 is substituted with either an A, C, D, E, F, G, H, I, K, L, M, N, P, Q, R, T, V, W, or Y; wherein R874 is substituted with either an A, C, D, E, F, G, H, I, K, L, M, N, P, Q, S, T, V, W, or Y; wherein V875 is substituted with either an A, C, D, E, F, G, H, I, K, L, M, N, P, Q, R, S, T, W, or Y; wherein R876 is substituted with either an A, C, D, E, F, G, H, I, K, L, M, N, P, Q, S, T, V, W, or Y; wherein R877 is substituted with either an A, C, D, E, F, G, H, I, K, L, M, N, P, Q, S, T, V, W, or Y; wherein H878 is substituted with either an A, C, D, E, F, G, I, K, L, M, N, P, Q, R, S, T, V, W, or Y; wherein K879 is substituted with either an A, C, D, E, F, G, H, I, L, M, N, P, Q, R, S, T, V, W, or Y; wherein H880 is substituted with either an A, C, D, E, F, G, I, K, L, M, N, P, Q, R, S, T, V, W, or Y; wherein S881 is substituted with either an A, C, D, E, F, G, H, I, K, L, M, N, P, Q, R, T, V, W, or Y; wherein S882 is substituted with either an A, C, D, E, F, G, H, I, K, L, M, N, P, Q, R, T, V, W, or Y; wherein E883 is substituted with either an A, C, D, F, G, H, I, K, L, M, N, P, Q, R, S, T, V, W, or Y; wherein S884 is substituted with either an A, C, D, E, F, G, H, I, K, L, M, N, P, Q, R, T, V, W, or Y; wherein P885 is substituted with either an A, C, D, E, F, G, H, I, K, L, M, N, Q, R, S, T, V, W, or Y; wherein G886 is substituted with either an A, C, D, E, F, H, I, K, L, M, N, P, Q, R, S, T, V, W, or Y; wherein R887 is substituted with either an A, C, D, E, F, G, H, I, K, L, M, N, P, Q, S, T, V, W, or Y; wherein D888 is substituted with either an A, C, E, F, G, H, I, K, L, M, N, P, Q, R, S, T, V, W, or Y; wherein K889 is substituted with either an A, C, D, E, F, G, H, I, L, M, N, P, Q, R, S, T, V, W, or Y; wherein G890 is substituted with either an A, C, D, E, F, H, I, K, L, M, N, P, Q, R, S, T, V, W, or Y; wherein K891 is substituted with either an A, C, D, E, F, G, H, I, L, M, N, P, Q, R, S, T, V, W, or Y; wherein L892 is substituted with either an A, C, D, E, F, G, H, I, K, M, N, P, Q, R, S, T, V, W, or Y; wherein S893 is substituted with either an A, C, D, E, F, G, H, I, K, L, M, N, P, Q, R, T, V, W, or Y; wherein K894 is substituted with either an A, C, D, E, F, G, H, I, L, M, N, P, Q, R, S, T, V, W, or Y; wherein L895 is substituted with either an A, C, D, E, F, G, H, I, K, M, N, P, Q, R, S, T, V, W, or Y; wherein K896 is substituted with either an A, C, D, E, F, G, H, I, L, M, N, P, Q, R, S, T, V, W, or Y; wherein P897 is substituted with either an A, C, D, E, F, G, H, I, K, L, M, N, Q, R, S, T, V, W, or Y; wherein A898 is substituted with either a C, D, E, F, G, H, I, K, L, M, N, P, Q, R, S, T, V, W, or Y; wherein P899 is substituted with either an A, C, D, E, F, G, H, I, K, L, M, N, Q, R, S, T, V, W, or Y; wherein P900 is substituted with either an A, C, D, E, F, G, H, I, K, L, M, N, Q, R, S, T, V, W, or Y; wherein P901 is substituted with either an A, C, D, E, F, G, H, I, K, L, M, N, Q, R, S, T, V, W, or Y; wherein P902 is substituted with either an A, C, D, E, F, G, H, I, K, L, M, N, Q, R, S, T, V, W, or Y; wherein P903 is substituted with either an A, C, D, E, F, G, H, I, K, L, M, N, Q, R, S, T, V, W, or Y; wherein A904 is substituted with either a C, D, E, F, G, H, I, K, L, M, N, P, Q, R, S, T, V, W, or Y; wherein A905 is substituted with either a C, D, E, F, G, H, I, K, L, M, N, P, Q, R, S, T, V, W, or Y; wherein S906 is substituted with either an A, C, D, E, F, G, H, I, K, L, M, N, P, Q, R, T, V, W, or Y; wherein A907 is substituted with either a C, D, E, F, G, H, I, K, L, M, N, P, Q, R, S, T, V, W, or Y; wherein G908 is substituted with either an A, C, D, E, F, H, I, K, L, M, N, P, Q, R, S, T, V, W, or Y; wherein K909 is substituted with either an A, C, D, E, F, G, H, I, L, M, N, P, Q, R, S, T, V, W, or Y; wherein A910 is substituted with either a C, D, E, F, G, H, I, K, L, M, N, P, Q, R, S, T, V, W, or Y; wherein G911 is substituted with either an A, C, D, E, F, H, I, K, L, M, N, P, Q, R, S, T, V, W, or Y; wherein G912 is substituted with either an A, C, D, E, F, H, I, K, L, M, N, P, Q, R, S, T, V, W, or Y; wherein K913 is substituted with either an A, C, D, E, F, G, H, I, L, M, N, P, Q, R, S, T, V, W, or Y; wherein P914 is substituted with either an A, C, D, E, F, G, H, I, K, L, M, N, Q, R, S, T, V, W, or Y; wherein S915 is substituted with either an A, C, D, E, F, G, H, I, K, L, M, N, P, Q, R, T, V, W, or Y; wherein Q916 is substituted with either an A, C, D, E, F, G, H, I, K, L, M, N, P, R, S, T, V, W, or Y; wherein R917 is substituted with either an A, C, D, E, F, G, H, I, K, L, M, N, P, Q, S, T, V, W, or Y; wherein P918 is substituted with either an A, C, D, E, F, G, H, I, K, L, M, N, Q, R, S, T, V, W, or Y; wherein G919 is substituted with either an A, C, D, E, F, H, I, K, L, M, N, P, Q, R, S, T, V, W, or Y; wherein Q920 is substituted with either an A, C, D, E, F, G, H, I, K, L, M, N, P, R, S, T, V, W, or Y; wherein E921 is substituted with either an A, C, D, F, G, H, I, K, L, M, N, P, Q, R, S, T, V, W, or Y; wherein A922 is substituted with either a C, D, E, F, G, H, I, K, L, M, N, P, Q, R, S, T, V, W, or Y; wherein A923 is substituted with either a C, D, E, F, G, H, I, K, L, M, N, P, Q, R, S, T, V, W, or Y; wherein G924 is substituted with either an A, C, D, E, F, H, I, K, L, M, N, P, Q, R, S, T, V, W, or Y; wherein E925 is substituted with either an A, C, D, F, G, H, I, K, L, M, N, P, Q, R, S, T, V, W, or Y; wherein A926 is substituted with either a C, D, E, F, G, H, I, K, L, M, N, P, Q, R, S, T, V, W, or Y; wherein V927 is substituted with either an A, C, D, E, F, G, H, I, K, L, M, N, P, Q, R, S, T, W, or Y; wherein L928 is substituted with either an A, C, D, E, F, G, H, I, K, M, N, P, Q, R, S, T, V, W, or Y; wherein G929 is substituted with either an A, C, D, E, F, H, I, K, L, M, N, P, Q, R, S, T, V, W, or Y; wherein A930 is substituted with either a C, D, E, F, G, H, I, K, L, M, N, P, Q, R, S, T, V, W, or Y; wherein K931 is substituted with either an A, C, D, E, F, G, H, I, L, M, N, P, Q, R, S, T, V, W, or Y; wherein T932 is substituted with either an A, C, D, E, F, G, H, I, K, L, M, N, P, Q, R, S, V, W, or Y; wherein K933 is substituted with either an A, C, D, E, F, G, H, I, L, M, N, P, Q, R, S, T, V, W, or Y; wherein A934 is substituted with either a C, D, E, F, G, H, I, K, L, M, N, P, Q, R, S, T, V, W, or Y; wherein T935 is substituted with either an A, C, D, E, F, G, H, I, K, L, M, N, P, Q, R, S, V, W, or Y; wherein S936 is substituted with either an A, C, D, E, F, G, H, I, K, L, M, N, P, Q, R, T, V, W, or Y; wherein L937 is substituted with either an A, C, D, E, F, G, H, I, K, M, N, P, Q, R, S, T, V, W, or Y; wherein V938 is substituted with either an A, C, D, E, F, G, H, I, K, L, M, N, P, Q, R, S, T, W, or Y; wherein D939 is substituted with either an A, C, E, F, G, H, I, K, L, M, N, P, Q, R, S, T, V, W, or Y; wherein A940 is substituted with either a C, D, E, F, G, H, I, K, L, M, N, P, Q, R, S, T, V, W, or Y; wherein V941 is substituted with either an A, C, D, E, F, G, H, I, K, L, M, N, P, Q, R, S, T, W, or Y; wherein N942 is substituted with either an A, C, D, E, F, G, H, I, K, L, M, P, Q, R, S, T, V, W, or Y; wherein S943 is substituted with either an A, C, D, E, F, G, H, I, K, L, M, N, P, Q, R, T, V, W, or Y; wherein D944 is substituted with either an A, C, E, F, G, H, I, K, L, M, N, P, Q, R, S, T, V, W, or Y; wherein A945 is substituted with either a C, D, E, F, G, H, I, K, L, M, N, P, Q, R, S, T, V, W, or Y; wherein A946 is substituted with either a C, D, E, F, G, H, I, K, L, M, N, P, Q, R, S, T, V, W, or Y; wherein K947 is substituted with either an A, C, D, E, F, G, H, I, L, M, N, P, Q, R, S, T, V, W, or Y; wherein P948 is substituted with either an A, C, D, E, F, G, H, I, K, L, M, N, Q, R, S, T, V, W, or Y; wherein S949 is substituted with either an A, C, D, E, F, G, H, I, K, L, M, N, P, Q, R, T, V, W, or Y; wherein Q950 is substituted with either an A, C, D, E, F, G, H, I, K, L, M, N, P, R, S, T, V, W, or Y; wherein P951 is substituted with either an A, C, D, E, F, G, H, I, K, L, M, N, Q, R, S, T, V, W, or Y; wherein A952 is substituted with either a C, D, E, F, G, H, I, K, L, M, N, P, Q, R, S, T, V, W, or Y; wherein E953 is substituted with either an A, C, D, F, G, H, I, K, L, M, N, P, Q, R, S, T, V, W, or Y; wherein G954 is substituted with either an A, C, D, E, F, H, I, K, L, M, N, P, Q, R, S, T, V, W, or Y; wherein L955 is substituted with either an A, C, D, E, F, G, H, I, K, M, N, P, Q, R, S, T, V, W, or Y; wherein K956 is substituted with either an A, C, D, E, F, G, H, I, L, M, N, P, Q, R, S, T, V, W, or Y; wherein K957 is substituted with either an A, C, D, E, F, G, H, I, L, M, N, P, Q, R, S, T, V, W, or Y; wherein P958 is substituted with either an A, C, D, E, F, G, H, I, K, L, M, N, Q, R, S, T, V, W, or Y; wherein V959 is substituted with either an A, C, D, E, F, G, H, I, K, L, M, N, P, Q, R, S, T, W, or Y; wherein L960 is substituted with either an A, C, D, E, F, G, H, I, K, M, N, P, Q, R, S, T, V, W, or Y; wherein P961 is substituted with either an A, C, D, E, F, G, H, I, K, L, M, N, Q, R, S, T, V, W, or Y; wherein A962 is substituted with either a C, D, E, F, G, H, I, K, L, M, N, P, Q, R, S, T, V, W, or Y; wherein T963 is substituted with either an A, C, D, E, F, G, H, I, K, L, M, N, P, Q, R, S, V, W, or Y; wherein P964 is substituted with either an A, C, D, E, F, G, H, I, K, L, M, N, Q, R, S, T, V, W, or Y; wherein K965 is substituted with either an A, C, D, E, F, G, H, I, L, M, N, P, Q, R, S, T, V, W, or Y; wherein P966 is substituted with either an A, C, D, E, F, G, H, I, K, L, M, N, Q, R, S, T, V, W, or Y; wherein H967 is substituted with either an A, C, D, E, F, G, I, K, L, M, N, P, Q, R, S, T, V, W, or Y; wherein P968 is substituted with either an A, C, D, E, F, G, H, I, K, L, M, N, Q, R, S, T, V, W, or Y; wherein A969 is substituted with either a C, D, E, F, G, H, I, K, L, M, N, P, Q, R, S, T, V, W, or Y; wherein K970 is substituted with either an A, C, D, E, F, G, H, I, L, M, N, P, Q, R, S, T, V, W, or Y; wherein P971 is substituted with either an A, C, D, E, F, G, H, I, K, L, M, N, Q, R, S, T, V, W, or Y; wherein S972 is substituted with either an A, C, D, E, F, G, H, I, K, L, M, N, P, Q, R, T, V, W, or Y; wherein G973 is substituted with either an A, C, D, E, F, H, I, K, L, M, N, P, Q, R, S, T, V, W, or Y; wherein T974 is substituted with either an A, C, D, E, F, G, H, I, K, L, M, N, P, Q, R, S, V, W, or Y; wherein P975 is substituted with either an A, C, D, E, F, G, H, I, K, L, M, N, Q, R, S, T, V, W, or Y; wherein I976 is substituted with either an A, C, D, E, F, G, H, K, L, M, N, P, Q, R, S, T, V, W, or Y; wherein S977 is substituted with either an A, C, D, E, F, G, H, I, K, L, M, N, P, Q, R, T, V, W, or Y; wherein P978 is substituted with either an A, C, D, E, F, G, H, I, K, L, M, N, Q, R, S, T, V, W, or Y; wherein A979 is substituted with either a C, D, E, F, G, H, I, K, L, M, N, P, Q, R, S, T, V, W, or Y; wherein P980 is substituted with either an A, C, D, E, F, G, H, I, K, L, M, N, Q, R, S, T, V, W, or Y; wherein V981 is substituted with either an A, C, D, E, F, G, H, I, K, L, M, N, P, Q, R, S, T, W, or Y; wherein P982 is substituted with either an A, C, D, E, F, G, H, I, K, L, M, N, Q, R, S, T, V, W, or Y; wherein L983 is substituted with either an A, C, D, E, F, G, H, I, K, M, N, P, Q, R, S, T, V, W, or Y; wherein S984 is substituted with either an A, C, D, E, F, G, H, I, K, L, M, N, P, Q, R, T, V, W, or Y; wherein T985 is substituted with either an A, C, D, E, F, G, H, I, K, L, M, N, P, Q, R, S, V, W, or Y; wherein L986 is substituted with either an A, C, D, E, F, G, H, I, K, M, N, P, Q, R, S, T, V, W, or Y; wherein P987 is substituted with either an A, C, D, E, F, G, H, I, K, L, M, N, Q, R, S, T, V, W, or Y; wherein S988 is substituted with either an A, C, D, E, F, G, H, I, K, L, M, N, P, Q, R, T, V, W, or Y; wherein A989 is substituted with either a C, D, E, F, G, H, I, K, L, M, N, P, Q, R, S, T, V, W, or Y; wherein S990 is substituted with either an A, C, D, E, F, G, H, I, K, L, M, N, P, Q, R, T, V, W, or Y; wherein S991 is substituted with either an A, C, D, E, F, G, H, I, K, L, M, N, P, Q, R, T, V, W, or Y; wherein A992 is substituted with either a C, D, E, F, G, H, I, K, L, M, N, P, Q, R, S, T, V, W, or Y; wherein L993 is substituted with either an A, C, D, E, F, G, H, I, K, M, N, P, Q, R, S, T, V, W, or Y; wherein A994 is substituted with either a C, D, E, F, G, H, I, K, L, M, N, P, Q, R, S, T, V, W, or Y; wherein G995 is substituted with either an A, C, D, E, F, H, I, K, L, M, N, P, Q, R, S, T, V, W, or Y; wherein D996 is substituted with either an A, C, E, F, G, H, I, K, L, M, N, P, Q, R, S, T, V, W, or Y; wherein Q997 is substituted with either an A, C, D, E, F, G, H, I, K, L, M, N, P, R, S, T, V, W, or Y; wherein P998 is substituted with either an A, C, D, E, F, G, H, I, K, L, M, N, Q, R, S, T, V, W, or Y; wherein S999 is substituted with either an A, C, D, E, F, G, H, I, K, L, M, N, P, Q, R, T, V, W, or Y; wherein S1000 is substituted with either an A, C, D, E, F, G, H, I, K, L, M, N, P, Q, R, T, V, W, or Y; wherein T1001 is substituted with either an A, C, D, E, F, G, H, I, K, L, M, N, P, Q, R, S, V, W, or Y; wherein A1002 is substituted with either a C, D, E, F, G, H, I, K, L, M, N, P, Q, R, S, T, V, W, or Y; wherein F1003 is substituted with either an A, C, D, E, G, H, I, K, L, M, N, P, Q, R, S, T, V, W, or Y; wherein I1004 is substituted with either an A, C, D, E, F, G, H, K, L, M, N, P, Q, R, S, T, V, W, or Y; wherein P1005 is substituted with either an A, C, D, E, F, G, H, I, K, L, M, N, Q, R, S, T, V, W, or Y; wherein L1006 is substituted with either an A, C, D, E, F, G, H, I, K, M, N, P, Q, R, S, T, V, W, or Y; wherein I1007 is substituted with either an A, C, D, E, F, G, H, K, L, M, N, P, Q, R, S, T, V, W, or Y; wherein S1008 is substituted with either an A, C, D, E, F, G, H, I, K, L, M, N, P, Q, R, T, V, W, or Y; wherein T1009 is substituted with either an A, C, D, E, F, G, H, I, K, L, M, N, P, Q, R, S, V, W, or Y; wherein R100 is substituted with either an A, C, D, E, F, G, H, I, K, L, M, N, P, Q, S, T, V, W, or Y; wherein V1011 is substituted with either an A, C, D, E, F, G, H, I, K, L, M, N, P, Q, R, S, T, W, or Y; wherein S1012 is substituted with either an A, C, D, E, F, G, H, I, K, L, M, N, P, Q, R, T, V, W, or Y; wherein L1013 is substituted with either an A, C, D, E, F, G, H, I, K, M, N, P, Q, R, S, T, V, W, or Y; wherein R1014 is substituted with either an A, C, D, E, F, G, H, I, K, L, M, N, P, Q, S, T, V, W, or Y; wherein K1015 is substituted with either an A, C, D, E, F, G, H, I, L, M, N, P, Q, R, S, T, V, W, or Y; wherein T1016 is substituted with either an A, C, D, E, F, G, H, I, K, L, M, N, P, Q, R, S, V, W, or Y; wherein R1017 is substituted with either an A, C, D, E, F, G, H, I, K, L, M, N, P, Q, S, T, V, W, or Y; wherein Q1018 is substituted with either an A, C, D, E, F, G, H, I, K, L, M, N, P, R, S, T, V, W, or Y; wherein P1019 is substituted with either an A, C, D, E, F, G, H, I, K, L, M, N, Q, R, S, T, V, W, or Y; wherein P1020 is substituted with either an A, C, D, E, F, G, H, I, K, L, M, N, Q, R, S, T, V, W, or Y; wherein E1021 is substituted with either an A, C, D, F, G, H, I, K, L, M, N, P, Q, R, S, T, V, W, or Y; wherein R1022 is substituted with either an A, C, D, E, F, G, H, I, K, L, M, N, P, Q, S, T, V, W, or Y; wherein A1023 is substituted with either a C, D, E, F, G, H, I, K, L, M, N, P, Q, R, S, T, V, W, or Y; wherein S1024 is substituted with either an A, C, D, E, F, G, H, I, K, L, M, N, P, Q, R, T, V, W, or Y; wherein G1025 is substituted with either an A, C, D, E, F, H, I, K, L, M, N, P, Q, R, S, T, V, W, or Y; wherein A1026 is substituted with either a C, D, E, F, G, H, I, K, L, M, N, P, Q, R, S, T, V, W, or Y; wherein I1027 is substituted with either an A, C, D, E, F, G, H, K, L, M, N, P, Q, R, S, T, V, W, or Y; wherein T1028 is substituted with either an A, C, D, E, F, G, H, I, K, L, M, N, P, Q, R, S, V, W, or Y; wherein K1029 is substituted with either an A, C, D, E, F, G, H, I, L, M, N, P, Q, R, S, T, V, W, or Y; wherein G1030 is substituted with either an A, C, D, E, F, H, I, K, L, M, N, P, Q, R, S, T, V, W, or Y; wherein V1031 is substituted with either an A, C, D, E, F, G, H, I, K, L, M, N, P, Q, R, S, T, W, or Y; wherein V1032 is substituted with either an A, C, D, E, F, G, H, I, K, L, M, N, P, Q, R, S, T, W, or Y; wherein L1033 is substituted with either an A, C, D, E, F, G, H, I, K, M, N, P, Q, R, S, T, V, W, or Y; wherein D1034 is substituted with either an A, C, E, F, G, H, I, K, L, M, N, P, Q, R, S, T, V, W, or Y; wherein S1035 is substituted with either an A, C, D, E, F, G, H, I, K, L, M, N, P, Q, R, T, V, W, or Y; wherein T1036 is substituted with either an A, C, D, E, F, G, H, I, K, L, M, N, P, Q, R, S, V, W, or Y; wherein E1037 is substituted with either an A, C, D, F, G, H, I, K, L, M, N, P, Q, R, S, T, V, W, or Y; wherein A1038 is substituted with either a C, D, E, F, G, H, I, K, L, M, N, P, Q, R, S, T, V, W, or Y; wherein L1039 is substituted with either an A, C, D, E, F, G, H, I, K, M, N, P, Q, R, S, T, V, W, or Y; wherein C1040 is substituted with either an A, D, E, F, G, H, I, K, L, M, N, P, Q, R, S, T, V, W, or Y; wherein L1041 is substituted with either an A, C, D, E, F, G, H, I, K, M, N, P, Q, R, S, T, V, W, or Y; wherein A1042 is substituted with either a C, D, E, F, G, H, I, K, L, M, N, P, Q, R, S, T, V, W, or Y; wherein I1043 is substituted with either an A, C, D, E, F, G, H, K, L, M, N, P, Q, R, S, T, V, W, or Y; wherein S1044 is substituted with either an A, C, D, E, F, G, H, I, K, L, M, N, P, Q, R, T, V, W, or Y; wherein G1045 is substituted with either an A, C, D, E, F, H, I, K, L, M, N, P, Q, R, S, T, V, W, or Y; wherein N1046 is substituted with either an A, C, D, E, F, G, H, I, K, L, M, P, Q, R, S, T, V, W, or Y; wherein S1047 is substituted with either an A, C, D, E, F, G, H, I, K, L, M, N, P, Q, R, T, V, W, or Y; wherein E1048 is substituted with either an A, C, D, F, G, H, I, K, L, M, N, P, Q, R, S, T, V, W, or Y; wherein Q1049 is substituted with either an A, C, D, E, F, G, H, I, K, L, M, N, P, R, S, T, V, W, or Y; wherein M1050 is substituted with either an A, C, D, E, F, G, H, I, K, L, N, P, Q, R, S, T, V, W, or Y; wherein A1051 is substituted with either a C, D, E, F, G, H, I, K, L, M, N, P, Q, R, S, T, V, W, or Y; wherein S1052 is substituted with either an A, C, D, E, F, G, H, I, K, L, M, N, P, Q, R, T, V, W, or Y; wherein H1053 is substituted with either an A, C, D, E, F, G, I, K, L, M, N, P, Q, R, S, T, V, W, or Y; wherein S1054 is substituted with either an A, C, D, E, F, G, H, I, K, L, M, N, P, Q, R, T, V, W, or Y; wherein A1055 is substituted with either a C, D, E, F, G, H, I, K, L, M, N, P, Q, R, S, T, V, W, or Y; wherein V1056 is substituted with either an A, C, D, E, F, G, H, I, K, L, M, N, P, Q, R, S, T, W, or Y; wherein L1057 is substituted with either an A, C, D, E, F, G, H, I, K, M, N, P, Q, R, S, T, V, W, or Y; wherein E1058 is substituted with either an A, C, D, F, G, H, I, K, L, M, N, P, Q, R, S, T, V, W, or Y; wherein A1059 is substituted with either a C, D, E, F, G, H, I, K, L, M, N, P, Q, R, S, T, V, W, or Y; wherein G1060 is substituted with either an A, C, D, E, F, H, I, K, L, M, N, P, Q, R, S, T, V, W, or Y; wherein K1061 is substituted with either an A, C, D, E, F, G, H, I, L, M, N, P, Q, R, S, T, V, W, or Y; wherein N1062 is substituted with either an A, C, D, E, F, G, H, I, K, L, M, P, Q, R, S, T, V, W, or Y; wherein L1063 is substituted with either an A, C, D, E, F, G, H, I, K, M, N, P, Q, R, S, T, V, W, or Y; wherein Y1064 is substituted with either an A, C, D, E, F, G, H, I, K, L, M, N, P, Q, R, S, T, V, or W; wherein T1065 is substituted with either an A, C, D, E, F, G, H, I, K, L, M, N, P, Q, R, S, V, W, or Y; wherein F1066 is substituted with either an A, C, D, E, G, H, I, K, L, M, N, P, Q, R, S, T, V, W, or Y; wherein C1067 is substituted with either an A, D, E, F, G, H, I, K, L, M, N, P, Q, R, S, T, V, W, or Y; wherein V1068 is substituted with either an A, C, D, E, F, G, H, I, K, L, M, N, P, Q, R, S, T, W, or Y; wherein S1069 is substituted with either an A, C, D, E, F, G, H, I, K, L, M, N, P, Q, R, T, V, W, or Y; wherein Y1070 is substituted with either an A, C, D, E, F, G, H, I, K, L, M, N, P, Q, R, S, T, V, or W; wherein V1071 is substituted with either an A, C, D, E, F, G, H, I, K, L, M, N, P, Q, R, S, T, W, or Y; wherein D1072 is substituted with either an A, C, E, F, G, H, I, K, L, M, N, P, Q, R, S, T, V, W, or Y; wherein S1073 is substituted with either an A, C, D, E, F, G, H, I, K, L, M, N, P, Q, R, T, V, W, or Y; wherein I1074 is substituted with either an A, C, D, E, F, G, H, K, L, M, N, P, Q, R, S, T, V, W, or Y; wherein Q1075 is substituted with either an A, C, D, E, F, G, H, I, K, L, M, N, P, R, S, T, V, W, or Y; wherein Q1076 is substituted with either an A, C, D, E, F, G, H, I, K, L, M, N, P, R, S, T, V, W, or Y; wherein M1077 is substituted with either an A, C, D, E, F, G, H, I, K, L, N, P, Q, R, S, T, V, W, or Y; wherein R1078 is substituted with either an A, C, D, E, F, G, H, I, K, L, M, N, P, Q, S, T, V, W, or Y; wherein N1079 is substituted with either an A, C, D, E, F, G, H, I, K, L, M, P, Q, R, S, T, V, W, or Y; wherein K1080 is substituted with either an A, C, D, E, F, G, H, I, L, M, N, P, Q, R, S, T, V, W, or Y; wherein F1081 is substituted with either an A, C, D, E, G, H, I, K, L, M, N, P, Q, R, S, T, V, W, or Y; wherein A1082 is substituted with either a C, D, E, F, G, H, I, K, L, M, N, P, Q, R, S, T, V, W, or Y; wherein F1083 is substituted with either an A, C, D, E, G, H, I, K, L, M, N, P, Q, R, S, T, V, W, or Y; wherein R1084 is substituted with either an A, C, D, E, F, G, H, I, K, L, M, N, P, Q, S, T, V, W, or Y; wherein E1085 is substituted with either an A, C, D, F, G, H, I, K, L, M, N, P, Q, R, S, T, V, W, or Y; wherein A1086 is substituted with either a C, D, E, F, G, H, I, K, L, M, N, P, Q, R, S, T, V, W, or Y; wherein I1087 is substituted with either an A, C, D, E, F, G, H, K, L, M, N, P, Q, R, S, T, V, W, or Y; wherein N1088 is substituted with either an A, C, D, E, F, G, H, I, K, L, M, P, Q, R, S, T, V, W, or Y; wherein K1089 is substituted with either an A, C, D, E, F, G, H, I, L, M, N, P, Q, R, S, T, V, W, or Y; wherein L1090 is substituted with either an A, C, D, E, F, G, H, I, K, M, N, P, Q, R, S, T, V, W, or Y; wherein E1091 is substituted with either an A, C, D, F, G, H, I, K, L, M, N, P, Q, R, S, T, V, W, or Y; wherein N1092 is substituted with either an A, C, D, E, F, G, H, I, K, L, M, P, Q, R, S, T, V, W, or Y; wherein N1093 is substituted with either an A, C, D, E, F, G, H, I, K, L, M, P, Q, R, S, T, V, W, or Y; wherein L1094 is substituted with either an A, C, D, E, F, G, H, I, K, M, N, P, Q, R, S, T, V, W, or Y; wherein R1095 is substituted with either an A, C, D, E, F, G, H, I, K, L, M, N, P, Q, S, T, V, W, or Y; wherein E1096 is substituted with either an A, C, D, F, G, H, I, K, L, M, N, P, Q, R, S, T, V, W, or Y; wherein L1097 is substituted with either an A, C, D, E, F, G, H, I, K, M, N, P, Q, R, S, T, V, W, or Y; wherein Q1098 is substituted with either an A, C, D, E, F, G, H, I, K, L, M, N, P, R, S, T, V, W, or Y; wherein I1099 is substituted with either an A, C, D, E, F, G, H, K, L, M, N, P, Q, R, S, T, V, W, or Y; wherein C1100 is substituted with either an A, D, E, F, G, H, I, K, L, M, N, P, Q, R, S, T, V, W, or Y; wherein P1101 is substituted with either an A, C, D, E, F, G, H, I, K, L, M, N, Q, R, S, T, V, W, or Y; wherein A1102 is substituted with either a C, D, E, F, G, H, I, K, L, M, N, P, Q, R, S, T, V, W, or Y; wherein S1103 is substituted with either an A, C, D, E, F, G, H, I, K, L, M, N, P, Q, R, T, V, W, or Y; wherein A1104 is substituted with either a C, D, E, F, G, H, I, K, L, M, N, P, Q, R, S, T, V, W, or Y; wherein G1105 is substituted with either an A, C, D, E, F, H, I, K, L, M, N, P, Q, R, S, T, V, W, or Y; wherein S1106 is substituted with either an A, C, D, E, F, G, H, I, K, L, M, N, P, Q, R, T, V, W, or Y; wherein G1107 is substituted with either an A, C, D, E, F, H, I, K, L, M, N, P, Q, R, S, T, V, W, or Y; wherein P1108 is substituted with either an A, C, D, E, F, G, H, I, K, L, M, N, Q, R, S, T, V, W, or Y; wherein A1109 is substituted with either a C, D, E, F, G, H, I, K, L, M, N, P, Q, R, S, T, V, W, or Y; wherein A1110 is substituted with either a C, D, E, F, G, H, I, K, L, M, N, P, Q, R, S, T, V, W, or Y; wherein T1111 is substituted with either an A, C, D, E, F, G, H, I, K, L, M, N, P, Q, R, S, V, W, or Y; wherein Q1112 is substituted with either an A, C, D, E, F, G, H, I, K, L, M, N, P, R, S, T, V, W, or Y; wherein D1113 is substituted with either an A, C, E, F, G, H, I, K, L, M, N, P, Q, R, S, T, V, W, or Y; wherein F1114 is substituted with either an A, C, D, E, G, H, I, K, L, M, N, P, Q, R, S, T, V, W, or Y; wherein S1115 is substituted with either an A, C, D, E, F, G, H, I, K, L, M, N, P, Q, R, T, V, W, or Y; wherein K1116 is substituted with either an A, C, D, E, F, G, H, I, L, M, N, P, Q, R, S, T, V, W, or Y; wherein L1117 is substituted with either an A, C, D, E, F, G, H, I, K, M, N, P, Q, R, S, T, V, W, or Y; wherein L1118 is substituted with either an A, C, D, E, F, G, H, I, K, M, N, P, Q, R, S, T, V, W, or Y; wherein S1119 is substituted with either an A, C, D, E, F, G, H, I, K, L, M, N, P, Q, R, T, V, W, or Y; wherein S1120 is substituted with either an A, C, D, E, F, G, H, I, K, L, M, N, P, Q, R, T, V, W, or Y; wherein V1121 is substituted with either an A, C, D, E, F, G, H, I, K, L, M, N, P, Q, R, S, T, W, or Y; wherein K1122 is substituted with either an A, C, D, E, F, G, H, I, L, M, N, P, Q, R, S, T, V, W, or Y; wherein E1123 is substituted with either an A, C, D, F, G, H, I, K, L, M, N, P, Q, R, S, T, V, W, or Y; wherein I1124 is substituted with either an A, C, D, E, F, G, H, K, L, M, N, P, Q, R, S, T, V, W, or Y; wherein S1125 is substituted with either an A, C, D, E, F, G, H, I, K, L, M, N, P, Q, R, T, V, W, or Y; wherein D1126 is substituted with either an A, C, E, F, G, H, I, K, L, M, N, P, Q, R, S, T, V, W, or Y; wherein I1127 is substituted with either an A, C, D, E, F, G, H, K, L, M, N, P, Q, R, S, T, V, W, or Y; wherein V1128 is substituted with either an A, C, D, E, F, G, H, I, K, L, M, N, P, Q, R, S, T, W, or Y; wherein Q1129 is substituted with either an A, C, D, E, F, G, H, I, K, L, M, N, P, R, S, T, V, W, or Y; and/or wherein R1130 is substituted with either an A, C, D, E, F, G, H, I, K, L, M, N, P, Q, S, T, V, W, or Y of SEQ ID NO:2, in addition to any combination thereof. The present invention also encompasses the use of these BCR/ABL amino acid substituted polypeptides as immunogenic and/or antigenic epitopes as described elsewhere herein.

In preferred embodiments, the following BCR/ABL conservative amino acid substitutions are encompassed by the present invention: wherein M1 is substituted with either an A, G, S, or T; wherein L2 is substituted with either an A, I, or V; wherein E3 is substituted with a D; wherein 14 is substituted with either an A, V, or L; wherein C5 is a C; wherein L6 is substituted with either an A, I, or V; wherein K7 is substituted with either a R, or H; wherein L8 is substituted with either an A, I, or V; wherein V9 is substituted with either an A, I, or L; wherein G10 is substituted with either an A, M, S, or T; wherein C11 is a C; wherein K12 is substituted with either a R, or H; wherein S13 is substituted with either an A, G, M, or T; wherein K14 is substituted with either a R, or H; wherein K15 is substituted with either a R, or H; wherein G16 is substituted with either an A, M, S, or T; wherein L17 is substituted with either an A, I, or V; wherein S18 is substituted with either an A, G, M, or T; wherein S19 is substituted with either an A, G, M, or T; wherein S20 is substituted with either an A, G, M, or T; wherein S21 is substituted with either an A, G, M, or T; wherein S22 is substituted with either an A, G, M, or T; wherein C23 is a C; wherein Y24 is either an F, or W; wherein L25 is substituted with either an A, I, or V; wherein E26 is substituted with a D; wherein E27 is substituted with a D; wherein A28 is substituted with either a G, I, L, M, S, T, or V; wherein L29 is substituted with either an A, I, or V; wherein Q30 is substituted with a N; wherein R31 is substituted with either a K, or H; wherein P32 is a P; wherein V33 is substituted with either an A, I, or L; wherein A34 is substituted with either a G, I, L, M, S, T, or V; wherein S35 is substituted with either an A, G, M, or T; wherein D36 is substituted with an E; wherein F37 is substituted with either a W, or Y; wherein E38 is substituted with a D; wherein P39 is a P; wherein Q40 is substituted with a N; wherein G41 is substituted with either an A, M, S, or T; wherein L42 is substituted with either an A, I, or V; wherein S43 is substituted with either an A, G, M, or T; wherein E44 is substituted with a D; wherein A45 is substituted with either a G, I, L, M, S, T, or V; wherein A46 is substituted with either a G, I, L, M, S, T, or V; wherein R47 is substituted with either a K, or H; wherein W48 is either an F, or Y; wherein N49 is substituted with a Q; wherein S50 is substituted with either an A, G, M, or T; wherein K51 is substituted with either a R, or H; wherein E52 is substituted with a D; wherein N53 is substituted with a Q; wherein L54 is substituted with either an A, I, or V; wherein L55 is substituted with either an A, I, or V; wherein A56 is substituted with either a G, I, L, M, S, T, or V; wherein G57 is substituted with either an A, M, S, or T; wherein P58 is a P; wherein S59 is substituted with either an A, G, M, or T; wherein E60 is substituted with a D; wherein N61 is substituted with a Q; wherein D62 is substituted with an E; wherein P63 is a P; wherein N64 is substituted with a Q; wherein L65 is substituted with either an A, I, or V; wherein F66 is substituted with either a W, or Y; wherein V67 is substituted with either an A, I, or L; wherein A68 is substituted with either a G, I, L, M, S, T, or V; wherein L69 is substituted with either an A, I, or V; wherein Y70 is either an F, or W; wherein D71 is substituted with an E; wherein F72 is substituted with either a W, or Y; wherein V73 is substituted with either an A, I, or L; wherein A74 is substituted with either a G, I, L, M, S, T, or V; wherein S75 is substituted with either an A, G, M, or T; wherein G76 is substituted with either an A, M, S, or T; wherein D77 is substituted with an E; wherein N78 is substituted with a Q; wherein T79 is substituted with either an A, G, M, or S; wherein L80 is substituted with either an A, I, or V; wherein S81 is substituted with either an A, G, M, or T; wherein I82 is substituted with either an A, V, or L; wherein T83 is substituted with either an A, G, M, or S; wherein K84 is substituted with either a R, or H; wherein G85 is substituted with either an A, M, S, or T; wherein E86 is substituted with a D; wherein K87 is substituted with either a R, or H; wherein L88 is substituted with either an A, I, or V; wherein R89 is substituted with either a K, or H; wherein V90 is substituted with either an A, I, or L; wherein L91 is substituted with either an A, I, or V; wherein G92 is substituted with either an A, M, S, or T; wherein Y93 is either an F, or W; wherein N94 is substituted with a Q; wherein H95 is substituted with either a K, or R; wherein N96 is substituted with a Q; wherein G97 is substituted with either an A, M, S, or T; wherein E98 is substituted with a D; wherein W99 is either an F, or Y; wherein C100 is a C; wherein E101 is substituted with a D; wherein A102 is substituted with either a G, I, L, M, S, T, or V; wherein Q103 is substituted with a N; wherein T104 is substituted with either an A, G, M, or S; wherein K105 is substituted with either a R, or H; wherein N106 is substituted with a Q; wherein G107 is substituted with either an A, M, S, or T; wherein Q108 is substituted with a N; wherein G109 is substituted with either an A, M, S, or T; wherein W110 is either an F, or Y; wherein V111 is substituted with either an A, I, or L; wherein P112 is a P; wherein S113 is substituted with either an A, G, M, or T; wherein N114 is substituted with a Q; wherein Y115 is either an F, or W; wherein I116 is substituted with either an A, V, or L; wherein T117 is substituted with either an A, G, M, or S; wherein P118 is a P; wherein V119 is substituted with either an A, I, or L; wherein N120 is substituted with a Q; wherein S121 is substituted with either an A, G, M, or T; wherein L122 is substituted with either an A, I, or V; wherein E123 is substituted with a D; wherein K124 is substituted with either a R, or H; wherein H125 is substituted with either a K, or R; wherein S126 is substituted with either an A, G, M, or T; wherein W127 is either an F, or Y; wherein Y128 is either an F, or W; wherein H129 is substituted with either a K, or R; wherein G130 is substituted with either an A, M, S, or T; wherein P131 is a P; wherein V132 is substituted with either an A, I, or L; wherein S133 is substituted with either an A, G, M, or T; wherein R134 is substituted with either a K, or H; wherein N135 is substituted with a Q; wherein A136 is substituted with either a G, I, L, M, S, T, or V; wherein A137 is substituted with either a G, I, L, M, S, T, or V; wherein E138 is substituted with a D; wherein Y139 is either an F, or W; wherein P140 is a P; wherein L141 is substituted with either an A, I, or V; wherein S142 is substituted with either an A, G, M, or T; wherein S143 is substituted with either an A, G, M, or T; wherein G144 is substituted with either an A, M, S, or T; wherein I145 is substituted with either an A, V, or L; wherein N146 is substituted with a Q; wherein G147 is substituted with either an A, M, S, or T; wherein S148 is substituted with either an A, G, M, or T; wherein F149 is substituted with either a W, or Y; wherein L150 is substituted with either an A, I, or V; wherein V151 is substituted with either an A, I, or L; wherein R152 is substituted with either a K, or H; wherein E153 is substituted with a D; wherein S154 is substituted with either an A, G, M, or T; wherein E155 is substituted with a D; wherein S156 is substituted with either an A, G, M, or T; wherein S157 is substituted with either an A, G, M, or T; wherein P158 is a P; wherein S159 is substituted with either an A, G, M, or T; wherein Q160 is substituted with a N; wherein R161 is substituted with either a K, or H; wherein S162 is substituted with either an A, G, M, or T; wherein I163 is substituted with either an A, V, or L; wherein S164 is substituted with either an A, G, M, or T; wherein L165 is substituted with either an A, I, or V; wherein R166 is substituted with either a K, or H; wherein Y167 is either an F, or W; wherein E168 is substituted with a D; wherein G169 is substituted with either an A, M, S, or T; wherein R170 is substituted with either a K, or H; wherein V171 is substituted with either an A, I, or L; wherein Y172 is either an F, or W; wherein H173 is substituted with either a K, or R; wherein Y174 is either an F, or W; wherein R175 is substituted with either a K, or H; wherein I176 is substituted with either an A, V, or L; wherein N177 is substituted with a Q; wherein T178 is substituted with either an A, G, M, or S; wherein A179 is substituted with either a G, I, L, M, S, T, or V; wherein S180 is substituted with either an A, G, M, or T; wherein D181 is substituted with an E; wherein G182 is substituted with either an A, M, S, or T; wherein K183 is substituted with either a R, or H; wherein L184 is substituted with either an A, I, or V; wherein Y185 is either an F, or W; wherein V186 is substituted with either an A, I, or L; wherein S187 is substituted with either an A, G, M, or T; wherein S188 is substituted with either an A, G, M, or T; wherein E189 is substituted with a D; wherein S190 is substituted with either an A, G, M, or T; wherein R191 is substituted with either a K, or H; wherein F192 is substituted with either a W, or Y; wherein N193 is substituted with a Q; wherein T194 is substituted with either an A, G, M, or S; wherein L195 is substituted with either an A, I, or V; wherein A196 is substituted with either a G, I, L, M, S, T, or V; wherein E197 is substituted with a D; wherein L198 is substituted with either an A, I, or V; wherein V199 is substituted with either an A, I, or L; wherein H200 is substituted with either a K, or R; wherein H201 is substituted with either a K, or R; wherein H202 is substituted with either a K, or R; wherein S203 is substituted with either an A, G, M, or T; wherein T204 is substituted with either an A, G, M, or S; wherein V205 is substituted with either an A, I, or L; wherein A206 is substituted with either a G, I, L, M, S, T, or V; wherein D207 is substituted with an E; wherein G208 is substituted with either an A, M, S, or T; wherein L209 is substituted with either an A, I, or V; wherein I210 is substituted with either an A, V, or L; wherein T211 is substituted with either an A, G, M, or S; wherein T212 is substituted with either an A, G, M, or S; wherein L213 is substituted with either an A, I, or V; wherein H214 is substituted with either a K, or R; wherein Y215 is either an F, or W; wherein P216 is a P; wherein A217 is substituted with either a G, I, L, M, S, T, or V; wherein P218 is a P; wherein K219 is substituted with either a R, or H; wherein R220 is substituted with either a K, or H; wherein N221 is substituted with a Q; wherein K222 is substituted with either a R, or H; wherein P223 is a P; wherein T224 is substituted with either an A, G, M, or S; wherein V225 is substituted with either an A, I, or L; wherein Y226 is either an F, or W; wherein G227 is substituted with either an A, M, S, or T; wherein V228 is substituted with either an A, I, or L; wherein S229 is substituted with either an A, G, M, or T; wherein P230 is a P; wherein N231 is substituted with a Q; wherein Y232 is either an F, or W; wherein D233 is substituted with an E; wherein K234 is substituted with either a R, or H; wherein W235 is either an F, or Y; wherein E236 is substituted with a D; wherein M237 is substituted with either an A, G, S, or T; wherein E238 is substituted with a D; wherein R239 is substituted with either a K, or H; wherein T240 is substituted with either an A, G, M, or S; wherein D241 is substituted with an E; wherein I242 is substituted with either an A, V, or L; wherein T243 is substituted with either an A, G, M, or S; wherein M244 is substituted with either an A, G, S, or T; wherein K245 is substituted with either a R, or H; wherein H246 is substituted with either a K, or R; wherein K247 is substituted with either a R, or H; wherein L248 is substituted with either an A, I, or V; wherein G249 is substituted with either an A, M, S, or T; wherein G250 is substituted with either an A, M, S, or T; wherein G251 is substituted with either an A, M, S, or T; wherein Q252 is substituted with a N; wherein Y253 is either an F, or W; wherein G254 is substituted with either an A, M, S, or T; wherein E255 is substituted with a D; wherein V256 is substituted with either an A, I, or L; wherein Y257 is either an F, or W; wherein E258 is substituted with a D; wherein G259 is substituted with either an A, M, S, or T; wherein V260 is substituted with either an A, I, or L; wherein W261 is either an F, or Y; wherein K262 is substituted with either a R, or H; wherein K263 is substituted with either a R, or H; wherein Y264 is either an F, or W; wherein S265 is substituted with either an A, G, M, or T; wherein L266 is substituted with either an A, I, or V; wherein T267 is substituted with either an A, G, M, or S; wherein V268 is substituted with either an A, I, or L; wherein A269 is substituted with either a G, I, L, M, S, T, or V; wherein V270 is substituted with either an A, I, or L; wherein K271 is substituted with either a R, or H; wherein T272 is substituted with either an A, G, M, or S; wherein L273 is substituted with either an A, I, or V; wherein K274 is substituted with either a R, or H; wherein E275 is substituted with a D; wherein D276 is substituted with an E; wherein T277 is substituted with either an A, G, M, or S; wherein M278 is substituted with either an A, G, S, or T; wherein E279 is substituted with a D; wherein V280 is substituted with either an A, I, or L; wherein E281 is substituted with a D; wherein E282 is substituted with a D; wherein F283 is substituted with either a W, or Y; wherein L284 is substituted with either an A, I, or V; wherein K285 is substituted with either a R, or H; wherein E286 is substituted with a D; wherein A287 is substituted with either a G, I, L, M, S, T, or V; wherein A288 is substituted with either a G, I, L, M, S, T, or V; wherein V289 is substituted with either an A, I, or L; wherein M290 is substituted with either an A, G, S, or T; wherein K291 is substituted with either a R, or H; wherein E292 is substituted with a D; wherein I293 is substituted with either an A, V, or L; wherein K294 is substituted with either a R, or H; wherein H295 is substituted with either a K, or R; wherein P296 is a P; wherein N297 is substituted with a Q; wherein L298 is substituted with either an A, I, or V; wherein V299 is substituted with either an A, I, or L; wherein Q300 is substituted with a N; wherein L301 is substituted with either an A, I, or V; wherein L302 is substituted with either an A, I, or V; wherein G303 is substituted with either an A, M, S, or T; wherein V304 is substituted with either an A, I, or L; wherein C305 is a C; wherein T306 is substituted with either an A, G, M, or S; wherein R307 is substituted with either a K, or H; wherein E308 is substituted with a D; wherein P309 is a P; wherein P310 is a P; wherein F311 is substituted with either a W, or Y; wherein Y312 is either an F, or W; wherein I313 is substituted with either an A, V, or L; wherein I314 is substituted with either an A, V, or L; wherein T315 is substituted with either an A, G, M, or S; wherein E316 is substituted with a D; wherein F317 is substituted with either a W, or Y; wherein M318 is substituted with either an A, G, S, or T; wherein T319 is substituted with either an A, G, M, or S; wherein Y320 is either an F, or W; wherein G321 is substituted with either an A, M, S, or T; wherein N322 is substituted with a Q; wherein L323 is substituted with either an A, I, or V; wherein L324 is substituted with either an A, I, or V; wherein D325 is substituted with an E; wherein Y326 is either an F, or W; wherein L327 is substituted with either an A, I, or V; wherein R328 is substituted with either a K, or H; wherein E329 is substituted with a D; wherein C330 is a C; wherein N331 is substituted with a Q; wherein R332 is substituted with either a K, or H; wherein Q333 is substituted with a N; wherein E334 is substituted with a D; wherein V335 is substituted with either an A, I, or L; wherein N336 is substituted with a Q; wherein A337 is substituted with either a G, I, L, M, S, T, or V; wherein V338 is substituted with either an A, I, or L; wherein V339 is substituted with either an A, I, or L; wherein L340 is substituted with either an A, I, or V; wherein L341 is substituted with either an A, I, or V; wherein Y342 is either an F, or W; wherein M343 is substituted with either an A, G, S, or T; wherein A344 is substituted with either a G, I, L, M, S, T, or V; wherein T345 is substituted with either an A, G, M, or S; wherein Q346 is substituted with a N; wherein I347 is substituted with either an A, V, or L; wherein S348 is substituted with either an A, G, M, or T; wherein S349 is substituted with either an A, G, M, or T; wherein A350 is substituted with either a G, I, L, M, S, T, or V; wherein M351 is substituted with either an A, G, S, or T; wherein E352 is substituted with a D; wherein Y353 is either an F, or W; wherein L354 is substituted with either an A, I, or V; wherein E355 is substituted with a D; wherein K356 is substituted with either a R, or H; wherein K357 is substituted with either a R, or H; wherein N358 is substituted with a Q; wherein F359 is substituted with either a W, or Y; wherein I360 is substituted with either an A, V, or L; wherein H361 is substituted with either a K, or R; wherein R362 is substituted with either a K, or H; wherein D363 is substituted with an E; wherein L364 is substituted with either an A, I, or V; wherein A365 is substituted with either a G, I, L, M, S, T, or V; wherein A366 is substituted with either a G, I, L, M, S, T, or V; wherein R367 is substituted with either a K, or H; wherein N368 is substituted with a Q; wherein C369 is a C; wherein L370 is substituted with either an A, I, or V; wherein V371 is substituted with either an A, I, or L; wherein G372 is substituted with either an A, M, S, or T; wherein E373 is substituted with a D; wherein N374 is substituted with a Q; wherein H375 is substituted with either a K, or R; wherein L376 is substituted with either an A, I, or V; wherein V377 is substituted with either an A, I, or L; wherein K378 is substituted with either a R, or H; wherein V379 is substituted with either an A, I, or L; wherein A380 is substituted with either a G, I, L, M, S, T, or V; wherein D381 is substituted with an E; wherein F382 is substituted with either a W, or Y; wherein G383 is substituted with either an A, M, S, or T; wherein L384 is substituted with either an A, I, or V; wherein S385 is substituted with either an A, G, M, or T; wherein R386 is substituted with either a K, or H; wherein L387 is substituted with either an A, I, or V; wherein M388 is substituted with either an A, G, S, or T; wherein T389 is substituted with either an A, G, M, or S; wherein G390 is substituted with either an A, M, S, or T; wherein D391 is substituted with an E; wherein T392 is substituted with either an A, G, M, or S; wherein Y393 is either an F, or W; wherein T394 is substituted with either an A, G, M, or S; wherein A395 is substituted with either a G, I, L, M, S, T, or V; wherein H396 is substituted with either a K, or R; wherein A397 is substituted with either a G, I, L, M, S, T, or V; wherein G398 is substituted with either an A, M, S, or T; wherein A399 is substituted with either a G, I, L, M, S, T, or V; wherein K400 is substituted with either a R, or H; wherein F401 is substituted with either a W, or Y; wherein P402 is a P; wherein I403 is substituted with either an A, V, or L; wherein K404 is substituted with either a R, or H; wherein W405 is either an F, or Y; wherein T406 is substituted with either an A, G, M, or S; wherein A407 is substituted with either a G, I, L, M, S, T, or V; wherein P408 is a P; wherein E409 is substituted with a D; wherein S410 is substituted with either an A, G, M, or T; wherein L411 is substituted with either an A, I, or V; wherein A412 is substituted with either a G, I, L, M, S, T, or V; wherein Y413 is either an F, or W; wherein N414 is substituted with a Q; wherein K415 is substituted with either a R, or H; wherein F416 is substituted with either a W, or Y; wherein S417 is substituted with either an A, G, M, or T; wherein I418 is substituted with either an A, V, or L; wherein K419 is substituted with either a R, or H; wherein S420 is substituted with either an A, G, M, or T; wherein D421 is substituted with an E; wherein V422 is substituted with either an A, I, or L; wherein W423 is either an F, or Y; wherein A424 is substituted with either a G, I, L, M, S, T, or V; wherein F425 is substituted with either a W, or Y; wherein G426 is substituted with either an A, M, S, or T; wherein V427 is substituted with either an A, I, or L; wherein L428 is substituted with either an A, I, or V; wherein L429 is substituted with either an A, I, or V; wherein W430 is either an F, or Y; wherein E431 is substituted with a D; wherein I432 is substituted with either an A, V, or L; wherein A433 is substituted with either a G, I, L, M, S, T, or V; wherein T434 is substituted with either an A, G, M, or S; wherein Y435 is either an F, or W; wherein G436 is substituted with either an A, M, S, or T; wherein M437 is substituted with either an A, G, S, or T; wherein S438 is substituted with either an A, G, M, or T; wherein P439 is a P; wherein Y440 is either an F, or W; wherein P441 is a P; wherein G442 is substituted with either an A, M, S, or T; wherein I443 is substituted with either an A, V, or L; wherein D444 is substituted with an E; wherein R445 is substituted with either a K, or H; wherein S446 is substituted with either an A, G, M, or T; wherein Q447 is substituted with a N; wherein V448 is substituted with either an A, I, or L; wherein Y449 is either an F, or W; wherein E450 is substituted with a D; wherein L451 is substituted with either an A, I, or V; wherein L452 is substituted with either an A, I, or V; wherein E453 is substituted with a D; wherein K454 is substituted with either a R, or H; wherein D455 is substituted with an E; wherein Y456 is either an F, or W; wherein R457 is substituted with either a K, or H; wherein M458 is substituted with either an A, G, S, or T; wherein K459 is substituted with either a R, or H; wherein R460 is substituted with either a K, or H; wherein P461 is a P; wherein E462 is substituted with a D; wherein G463 is substituted with either an A, M, S, or T; wherein C464 is a C; wherein P465 is a P; wherein E466 is substituted with a D; wherein K467 is substituted with either a R, or H; wherein V468 is substituted with either an A, I, or L; wherein Y469 is either an F, or W; wherein E470 is substituted with a D; wherein L471 is substituted with either an A, I, or V; wherein M472 is substituted with either an A, G, S, or T; wherein R473 is substituted with either a K, or H; wherein A474 is substituted with either a G, I, L, M, S, T, or V; wherein C475 is a C; wherein W476 is either an F, or Y; wherein Q477 is substituted with a N; wherein W478 is either an F, or Y; wherein N479 is substituted with a Q; wherein P480 is a P; wherein S481 is substituted with either an A, G, M, or T; wherein D482 is substituted with an E; wherein R483 is substituted with either a K, or H; wherein P484 is a P; wherein S485 is substituted with either an A, G, M, or T; wherein F486 is substituted with either a W, or Y; wherein A487 is substituted with either a G, I, L, M, S, T, or V; wherein E488 is substituted with a D; wherein I489 is substituted with either an A, V, or L; wherein H490 is substituted with either a K, or R; wherein Q491 is substituted with a N; wherein A492 is substituted with either a G, I, L, M, S, T, or V; wherein F493 is substituted with either a W, or Y; wherein E494 is substituted with a D; wherein T495 is substituted with either an A, G, M, or S; wherein M496 is substituted with either an A, G, S, or T; wherein F497 is substituted with either a W, or Y; wherein Q498 is substituted with a N; wherein E499 is substituted with a D; wherein S500 is substituted with either an A, G, M, or T; wherein S501 is substituted with either an A, G, M, or T; wherein I502 is substituted with either an A, V, or L; wherein S503 is substituted with either an A, G, M, or T; wherein D504 is substituted with an E; wherein E505 is substituted with a D; wherein V506 is substituted with either an A, I, or L; wherein E507 is substituted with a D; wherein K508 is substituted with either a R, or H; wherein E509 is substituted with a D; wherein L510 is substituted with either an A, I, or V; wherein G511 is substituted with either an A, M, S, or T; wherein K512 is substituted with either a R, or H; wherein Q513 is substituted with a N; wherein G514 is substituted with either an A, M, S, or T; wherein V515 is substituted with either an A, I, or L; wherein R516 is substituted with either a K, or H; wherein G517 is substituted with either an A, M, S, or T; wherein A518 is substituted with either a G, I, L, M, S, T, or V; wherein V519 is substituted with either an A, I, or L; wherein T520 is substituted with either an A, G, M, or S; wherein T521 is substituted with either an A, G, M, or S; wherein L522 is substituted with either an A, I, or V; wherein L523 is substituted with either an A, I, or V; wherein Q524 is substituted with a N; wherein A525 is substituted with either a G, I, L, M, S, T, or V; wherein P526 is a P; wherein E527 is substituted with a D; wherein L528 is substituted with either an A, I, or V; wherein P529 is a P; wherein T530 is substituted with either an A, G, M, or S; wherein K531 is substituted with either a R, or H; wherein T532 is substituted with either an A, G, M, or S; wherein R533 is substituted with either a K, or H; wherein T534 is substituted with either an A, G, M, or S; wherein S535 is substituted with either an A, G, M, or T; wherein R536 is substituted with either a K, or H; wherein R537 is substituted with either a K, or H; wherein A538 is substituted with either a G, I, L, M, S, T, or V; wherein A539 is substituted with either a G, I, L, M, S, T, or V; wherein E540 is substituted with a D; wherein H541 is substituted with either a K, or R; wherein R542 is substituted with either a K, or H; wherein D543 is substituted with an E; wherein T544 is substituted with either an A, G, M, or S; wherein T545 is substituted with either an A, G, M, or S; wherein D546 is substituted with an E; wherein V547 is substituted with either an A, I, or L; wherein P548 is a P; wherein E549 is substituted with a D; wherein M550 is substituted with either an A, G, S, or T; wherein P551 is a P; wherein H552 is substituted with either a K, or R; wherein S553 is substituted with either an A, G, M, or T; wherein K554 is substituted with either a R, or H; wherein G555 is substituted with either an A, M, S, or T; wherein Q556 is substituted with a N; wherein G557 is substituted with either an A, M, S, or T; wherein E558 is substituted with a D; wherein S559 is substituted with either an A, G, M, or T; wherein D560 is substituted with an E; wherein P561 is a P; wherein L562 is substituted with either an A, I, or V; wherein D563 is substituted with an E; wherein H564 is substituted with either a K, or R; wherein E565 is substituted with a D; wherein P566 is a P; wherein A567 is substituted with either a G, I, L, M, S, T, or V; wherein V568 is substituted with either an A, I, or L; wherein S569 is substituted with either an A, G, M, or T; wherein P570 is a P; wherein L571 is substituted with either an A, I, or V; wherein L572 is substituted with either an A, I, or V; wherein P573 is a P; wherein R574 is substituted with either a K, or H; wherein K575 is substituted with either a R, or H; wherein E576 is substituted with a D; wherein R577 is substituted with either a K, or H; wherein G578 is substituted with either an A, M, S, or T; wherein P579 is a P; wherein P580 is a P; wherein E581 is substituted with a D; wherein G582 is substituted with either an A, M, S, or T; wherein G583 is substituted with either an A, M, S, or T; wherein L584 is substituted with either an A, I, or V; wherein N585 is substituted with a Q; wherein E586 is substituted with a D; wherein D587 is substituted with an E; wherein E588 is substituted with a D; wherein R589 is substituted with either a K, or H; wherein L590 is substituted with either an A, I, or V; wherein L591 is substituted with either an A, I, or V; wherein P592 is a P; wherein K593 is substituted with either a R, or H; wherein D594 is substituted with an E; wherein K595 is substituted with either a R, or H; wherein K596 is substituted with either a R, or H; wherein T597 is substituted with either an A, G, M, or S; wherein N598 is substituted with a Q; wherein L599 is substituted with either an A, I, or V; wherein F600 is substituted with either a W, or Y; wherein S601 is substituted with either an A, G, M, or T; wherein A602 is substituted with either a G, I, L, M, S, T, or V; wherein L603 is substituted with either an A, I, or V; wherein I604 is substituted with either an A, V, or L; wherein K605 is substituted with either a R, or H; wherein K606 is substituted with either a R, or H; wherein K607 is substituted with either a R, or H; wherein K608 is substituted with either a R, or H; wherein K609 is substituted with either a R, or H; wherein T610 is substituted with either an A, G, M, or S; wherein A611 is substituted with either a G, I, L, M, S, T, or V; wherein P612 is a P; wherein T613 is substituted with either an A, G, M, or S; wherein P614 is a P; wherein P615 is a P; wherein K616 is substituted with either a R, or H; wherein R617 is substituted with either a K, or H; wherein S618 is substituted with either an A, G, M, or T; wherein S619 is substituted with either an A, G, M, or T; wherein S620 is substituted with either an A, G, M, or T; wherein F621 is substituted with either a W, or Y; wherein R622 is substituted with either a K, or H; wherein E623 is substituted with a D; wherein M624 is substituted with either an A, G, S, or T; wherein D625 is substituted with an E; wherein G626 is substituted with either an A, M, S, or T; wherein Q627 is substituted with a N; wherein P628 is a P; wherein E629 is substituted with a D; wherein R630 is substituted with either a K, or H; wherein R631 is substituted with either a K, or H; wherein G632 is substituted with either an A, M, S, or T; wherein A633 is substituted with either a G, I, L, M, S, T, or V; wherein G634 is substituted with either an A, M, S, or T; wherein E635 is substituted with a D; wherein E636 is substituted with a D; wherein E637 is substituted with a D; wherein G638 is substituted with either an A, M, S, or T; wherein R639 is substituted with either a K, or H; wherein D640 is substituted with an E; wherein I641 is substituted with either an A, V, or L; wherein S642 is substituted with either an A, G, M, or T; wherein N643 is substituted with a Q; wherein G644 is substituted with either an A, M, S, or T; wherein A645 is substituted with either a G, I, L, M, S, T, or V; wherein L646 is substituted with either an A, I, or V; wherein A647 is substituted with either a G, I, L, M, S, T, or V; wherein F648 is substituted with either a W, or Y; wherein T649 is substituted with either an A, G, M, or S; wherein P650 is a P; wherein L651 is substituted with either an A, I, or V; wherein D652 is substituted with an E; wherein T653 is substituted with either an A, G, M, or S; wherein A654 is substituted with either a G, I, L, M, S, T, or V; wherein D655 is substituted with an E; wherein P656 is a P; wherein A657 is substituted with either a G, I, L, M, S, T, or V; wherein K658 is substituted with either a R1 or H; wherein S659 is substituted with either an A, G, M, or T; wherein P660 is a P; wherein K661 is substituted with either a R, or H; wherein P662 is a P; wherein S663 is substituted with either an A, G, M, or T; wherein N664 is substituted with a Q; wherein G665 is substituted with either an A, M, S, or T; wherein A666 is substituted with either a G, I, L, M, S, T, or V; wherein G667 is substituted with either an A, M, S, or T; wherein V668 is substituted with either an A, I, or L; wherein P669 is a P; wherein N670 is substituted with a Q; wherein G671 is substituted with either an A, M, S, or T; wherein A672 is substituted with either a G, I, L, M, S, T, or V; wherein L673 is substituted with either an A, I, or V; wherein R674 is substituted with either a K, or H; wherein E675 is substituted with a D; wherein S676 is substituted with either an A, G, M, or T; wherein G677 is substituted with either an A, M, S, or T; wherein G678 is substituted with either an A, M, S, or T; wherein S679 is substituted with either an A, G, M, or T; wherein G680 is substituted with either an A, M, S, or T; wherein F681 is substituted with either a W, or Y; wherein R682 is substituted with either a K, or H; wherein S683 is substituted with either an A, G, M, or T; wherein P684 is a P; wherein H685 is substituted with either a K, or R; wherein L686 is substituted with either an A, I, or V; wherein W687 is either an F, or Y; wherein K688 is substituted with either a R, or H; wherein K689 is substituted with either a R, or H; wherein S690 is substituted with either an A, G, M, or T; wherein S691 is substituted with either an A, G, M, or T; wherein T692 is substituted with either an A, G, M, or S; wherein L693 is substituted with either an A, I, or V; wherein T694 is substituted with either an A, G, M, or S; wherein S695 is substituted with either an A, G, M, or T; wherein S696 is substituted with either an A, G, M, or T; wherein R697 is substituted with either a K, or H; wherein L698 is substituted with either an A, I, or V; wherein A699 is substituted with either a G, I, L, M, S, T, or V; wherein T700 is substituted with either an A, G, M, or S; wherein G701 is substituted with either an A, M, S, or T; wherein E702 is substituted with a D; wherein E703 is substituted with a D; wherein E704 is substituted with a D; wherein G705 is substituted with either an A, M, S, or T; wherein G706 is substituted with either an A, M, S, or T; wherein G707 is substituted with either an A, M, S, or T; wherein S708 is substituted with either an A, G, M, or T; wherein S709 is substituted with either an A, G, M, or T; wherein S710 is substituted with either an A, G, M, or T; wherein K711 is substituted with either a R, or H; wherein R712 is substituted with either a K, or H; wherein F713 is substituted with either a W, or Y; wherein L714 is substituted with either an A, I, or V; wherein R715 is substituted with either a K, or H; wherein S716 is substituted with either an A, G, M, or T; wherein C717 is a C; wherein S718 is substituted with either an A, G, M, or T; wherein V719 is substituted with either an A, I, or L; wherein S720 is substituted with either an A, G, M, or T; wherein C721 is a C; wherein V722 is substituted with either an A, I, or L; wherein P723 is a P; wherein H724 is substituted with either a K, or R; wherein G725 is substituted with either an A, M, S, or T; wherein A726 is substituted with either a G, I, L, M, S, T, or V; wherein K727 is substituted with either a R, or H; wherein D728 is substituted with an E; wherein T729 is substituted with either an A, G, M, or S; wherein E730 is substituted with a D; wherein W731 is either an F, or Y; wherein R732 is substituted with either a K, or H; wherein S733 is substituted with either an A, G, M, or T; wherein V734 is substituted with either an A, I, or L; wherein T735 is substituted with either an A, G, M, or S; wherein L736 is substituted with either an A, I, or V; wherein P737 is a P; wherein R738 is substituted with either a K, or H; wherein D739 is substituted with an E; wherein L740 is substituted with either an A, I, or V; wherein Q741 is substituted with a N; wherein S742 is substituted with either an A, G, M, or T; wherein T743 is substituted with either an A, G, M, or S; wherein G744 is substituted with either an A, M, S, or T; wherein R745 is substituted with either a K, or H; wherein Q746 is substituted with a N; wherein F747 is substituted with either a W, or Y; wherein D748 is substituted with an E; wherein S749 is substituted with either an A, G, M, or T; wherein S750 is substituted with either an A, G, M, or T; wherein T751 is substituted with either an A, G, M, or S; wherein F752 is substituted with either a W, or Y; wherein G753 is substituted with either an A, M, S, or T; wherein G754 is substituted with either an A, M, S, or T; wherein H755 is substituted with either a K, or R; wherein K756 is substituted with either a R, or H; wherein S757 is substituted with either an A, G, M, or T; wherein E758 is substituted with a D; wherein K759 is substituted with either a R, or H; wherein P760 is a P; wherein A761 is substituted with either a G, I, L, M, S, T, or V; wherein L762 is substituted with either an A, I, or V; wherein P763 is a P; wherein R764 is substituted with either a K, or H; wherein K765 is substituted with either a R, or H; wherein R766 is substituted with either a K, or H; wherein A767 is substituted with either a G, I, L, M, S, T, or V; wherein G768 is substituted with either an A, M, S, or T; wherein E769 is substituted with a D; wherein N770 is substituted with a Q; wherein R771 is substituted with either a K, or H; wherein S772 is substituted with either an A, G, M, or T; wherein D773 is substituted with an E; wherein Q774 is substituted with a N; wherein V775 is substituted with either an A, I, or L; wherein T776 is substituted with either an A, G, M, or S; wherein R777 is substituted with either a K, or H; wherein G778 is substituted with either an A, M, S, or T; wherein T779 is substituted with either an A, G, M, or S; wherein V780 is substituted with either an A, I, or L; wherein T781 is substituted with either an A, G, M, or S; wherein P782 is a P; wherein P783 is a P; wherein P784 is a P; wherein R785 is substituted with either a K, or H; wherein L786 is substituted with either an A, I, or V; wherein V787 is substituted with either an A, I, or L; wherein K788 is substituted with either a R, or H; wherein K789 is substituted with either a R, or H; wherein N790 is substituted with a Q; wherein E791 is substituted with a D; wherein E792 is substituted with a D; wherein A793 is substituted with either a G, I, L, M, S, T, or V; wherein A794 is substituted with either a G, I, L, M, S, T, or V; wherein D795 is substituted with an E; wherein E796 is substituted with a D; wherein V797 is substituted with either an A, I, or L; wherein F798 is substituted with either a W, or Y; wherein K799 is substituted with either a R, or H; wherein D800 is substituted with an E; wherein I801 is substituted with either an A, V, or L; wherein M802 is substituted with either an A, G, S, or T; wherein E803 is substituted with a D; wherein S804 is substituted with either an A, G, M, or T; wherein S805 is substituted with either an A, G, M, or T; wherein P806 is a P; wherein G807 is substituted with either an A, M, S, or T; wherein S808 is substituted with either an A, G, M, or T; wherein S809 is substituted with either an A, G, M, or T; wherein P810 is a P; wherein P811 is a P; wherein N812 is substituted with a Q; wherein L813 is substituted with either an A, I, or V; wherein T814 is substituted with either an A, G, M, or S; wherein P815 is a P; wherein K816 is substituted with either a R, or H; wherein P817 is a P; wherein L818 is substituted with either an A, I, or V; wherein R819 is substituted with either a K, or H; wherein R820 is substituted with either a K, or H; wherein Q821 is substituted with a N; wherein V822 is substituted with either an A, I, or L; wherein T823 is substituted with either an A, G, M, or S; wherein V824 is substituted with either an A, I, or L; wherein A825 is substituted with either a G, I, L, M, S, T, or V; wherein P826 is a P; wherein A827 is substituted with either a G, I, L, M, S, T, or V; wherein S828 is substituted with either an A, G, M, or T; wherein G829 is substituted with either an A, M, S, or T; wherein L830 is substituted with either an A, I, or V; wherein P831 is a P; wherein H832 is substituted with either a K, or R; wherein K833 is substituted with either a R, or H; wherein E834 is substituted with a D; wherein E835 is substituted with a D; wherein A836 is substituted with either a G, I, L, M, S, T, or V; wherein W837 is either an F, or Y; wherein K838 is substituted with either a R, or H; wherein G839 is substituted with either an A, M, S, or T; wherein S840 is substituted with either an A, G, M, or T; wherein A841 is substituted with either a G, I, L, M, S, T, or V; wherein L842 is substituted with either an A, I, or V; wherein G843 is substituted with either an A, M, S, or T; wherein T844 is substituted with either an A, G, M, or S; wherein P845 is a P; wherein A846 is substituted with either a G, I, L, M, S, T, or V; wherein A847 is substituted with either a G, I, L, M, S, T, or V; wherein A848 is substituted with either a G, I, L, M, S, T, or V; wherein E849 is substituted with a D; wherein P850 is a P; wherein V851 is substituted with either an A, I, or L; wherein T852 is substituted with either an A, G, M, or S; wherein P853 is a P; wherein T854 is substituted with either an A, G, M, or S; wherein S855 is substituted with either an A, G, M, or T; wherein K856 is substituted with either a R, or H; wherein A857 is substituted with either a G, I, L, M, S, T, or V; wherein G858 is substituted with either an A, M, S, or T; wherein S859 is substituted with either an A, G, M, or T; wherein G860 is substituted with either an A, M, S, or T; wherein A861 is substituted with either a G, I, L, M, S, T, or V; wherein P862 is a P; wherein R863 is substituted with either a K, or H; wherein G864 is substituted with either an A, M, S, or T; wherein T865 is substituted with either an A, G, M, or S; wherein S866 is substituted with either an A, G, M, or T; wherein K867 is substituted with either a R, or H; wherein G868 is substituted with either an A, M, S, or T; wherein P869 is a P; wherein A870 is substituted with either a G, I, L, M, S, T, or V; wherein E871 is substituted with a D; wherein E872 is substituted with a D; wherein S873 is substituted with either an A, G, M, or T; wherein R874 is substituted with either a K, or H; wherein V875 is substituted with either an A, I, or L; wherein R876 is substituted with either a K, or H; wherein R877 is substituted with either a K, or H; wherein H878 is substituted with either a K, or R; wherein K879 is substituted with either a R, or H; wherein H880 is substituted with either a K, or R; wherein S881 is substituted with either an A, G, M, or T; wherein S882 is substituted with either an A, G, M, or T; wherein E883 is substituted with a D; wherein S884 is substituted with either an A, G, M, or T; wherein P885 is a P; wherein G886 is substituted with either an A, M, S, or T; wherein R887 is substituted with either a K, or H; wherein D888 is substituted with an E; wherein K889 is substituted with either a R, or H; wherein G890 is substituted with either an A, M, S, or T; wherein K891 is substituted with either a R, or H; wherein L892 is substituted with either an A, I, or V; wherein S893 is substituted with either an A, G, M, or T; wherein K894 is substituted with either a R, or H; wherein L895 is substituted with either an A, I, or V; wherein K896 is substituted with either a R, or H; wherein P897 is a P; wherein A898 is substituted with either a G, I, L, M, S, T, or V; wherein P899 is a P; wherein P900 is a P; wherein P901 is a P; wherein P902 is a P; wherein P903 is a P; wherein A904 is substituted with either a G, I, L, M, S, T, or V; wherein A905 is substituted with either a G, I, L, M, S, T, or V; wherein S906 is substituted with either an A, G, M, or T; wherein A907 is substituted with either a G, I, L, M, S, T, or V; wherein G908 is substituted with either an A, M, S, or T; wherein K909 is substituted with either a R, or H; wherein A910 is substituted with either a G, I, L, M, S, T, or V; wherein G911 is substituted with either an A, M, S, or T; wherein G912 is substituted with either an A, M, S, or T; wherein K913 is substituted with either a R, or H; wherein P914 is a P; wherein S915 is substituted with either an A, G, M, or T; wherein Q916 is substituted with a N; wherein R917 is substituted with either a K, or H; wherein P918 is a P; wherein G919 is substituted with either an A, M, S, or T; wherein Q920 is substituted with a N; wherein E921 is substituted with a D; wherein A922 is substituted with either a G, I, L, M, S, T, or V; wherein A923 is substituted with either a G, I, L, M, S, T, or V; wherein G924 is substituted with either an A, M, S, or T; wherein E925 is substituted with a D; wherein A926 is substituted with either a G, I, L, M, S, T, or V; wherein V927 is substituted with either an A, I, or L; wherein L928 is substituted with either an A, I, or V; wherein G929 is substituted with either an A, M, S, or T; wherein A930 is substituted with either a G, I, L, M, S, T, or V; wherein K931 is substituted with either a R, or H; wherein T932 is substituted with either an A, G, M, or S; wherein K933 is substituted with either a R, or H; wherein A934 is substituted with either a G, I, L, M, S, T, or V; wherein T935 is substituted with either an A, G, M, or S; wherein S936 is substituted with either an A, G, M, or T; wherein L937 is substituted with either an A, I, or V; wherein V938 is substituted with either an A, I, or L; wherein D939 is substituted with an E; wherein A940 is substituted with either a G, I, L, M, S, T, or V; wherein V941 is substituted with either an A, I, or L; wherein N942 is substituted with a Q; wherein S943 is substituted with either an A, G, M, or T; wherein D944 is substituted with an E; wherein A945 is substituted with either a G, I, L, M, S, T, or V; wherein A946 is substituted with either a G, I, L, M, S, T, or V; wherein K947 is substituted with either a R, or H; wherein P948 is a P; wherein S949 is substituted with either an A, G, M, or T; wherein Q950 is substituted with a N; wherein P951 is a P; wherein A952 is substituted with either a G I, L, M, S, T, or V; wherein E953 is substituted with a D; wherein G954 is substituted with either an A, M, S, or T; wherein L955 is substituted with either an A, I, or V; wherein K956 is substituted with either a R, or H; wherein K957 is substituted with either a R, or H; wherein P958 is a P; wherein V959 is substituted with either an A, I, or L; wherein L960 is substituted with either an A, I, or V; wherein P961 is a P; wherein A962 is substituted with either a G, I, L, M, S, T, or V; wherein T963 is substituted with either an A, G, M, or S; wherein P964 is a P; wherein K965 is substituted with either a R, or H; wherein P966 is a P; wherein H967 is substituted with either a K, or R; wherein P968 is a P; wherein A969 is substituted with either a G, I, L, M, S, T, or V; wherein K970 is substituted with either a R, or H; wherein P971 is a P; wherein S972 is substituted with either an A, G, M, or T; wherein G973 is substituted with either an A, M, S, or T; wherein T974 is substituted with either an A, G, M, or S; wherein P975 is a P; wherein I976 is substituted with either an A, V, or L; wherein S977 is substituted with either an A, G, M, or T; wherein P978 is a P; wherein A979 is substituted with either a G, I, L, M, S, T, or V; wherein P980 is a P; wherein V981 is substituted with either an A, I, or L; wherein P982 is a P; wherein L983 is substituted with either an A, I, or V; wherein S984 is substituted with either an A, G, M, or T; wherein T985 is substituted with either an A, G, M, or S; wherein L986 is substituted with either an A, I, or V; wherein P987 is a P; wherein S988 is substituted with either an A, G, M, or T; wherein A989 is substituted with either a G, I, L, M, S, T, or V; wherein S990 is substituted with either an A, G, M, or T; wherein S991 is substituted with either an A, G, M, or T; wherein A992 is substituted with either a G, I, L, M, S, T, or V; wherein L993 is substituted with either an A, I, or V; wherein A994 is substituted with either a G, I, L, M, S, T, or V; wherein G995 is substituted with either an A, M, S, or T; wherein D996 is substituted with an E; wherein Q997 is substituted with a N; wherein P998 is a P; wherein S999 is substituted with either an A, G, M, or T; wherein S1000 is substituted with either an A, G, M, or T; wherein T1001 is substituted with either an A, G, M, or S; wherein A1002 is substituted with either a G, I, L, M, S, T, or V; wherein F1003 is substituted with either a W, or Y; wherein I1004 is substituted with either an A, V, or L; wherein P1005 is a P; wherein L1006 is substituted with either an A, I, or V; wherein I1007 is substituted with either an A, V, or L; wherein S1008 is substituted with either an A, G, M, or T; wherein T1009 is substituted with either an A, G, M, or S; wherein R1010 is substituted with either a K, or H; wherein V1011 is substituted with either an A, I, or L; wherein S1012 is substituted with either an A, G, M, or T; wherein L1013 is substituted with either an A, I, or V; wherein R1014 is substituted with either a K, or H; wherein K1015 is substituted with either a R, or H; wherein T1016 is substituted with either an A, G, M, or S; wherein R1017 is substituted with either a K, or H; wherein Q1018 is substituted with a N; wherein P1019 is a P; wherein P1020 is a P; wherein E1021 is substituted with a D; wherein R1022 is substituted with either a K, or H; wherein A1023 is substituted with either a G, I, L, M, S, T, or V; wherein S1024 is substituted with either an A, G, M, or T; wherein G1025 is substituted with either an A, M, S, or T; wherein A1026 is substituted with either a G, I, L, M, S, T, or V; wherein I1027 is substituted with either an A, V, or L; wherein T1028 is substituted with either an A, G, M, or S; wherein K1029 is substituted with either a R, or H; wherein G1030 is substituted with either an A, M, S, or T; wherein V1031 is substituted with either an A, I, or L; wherein V1032 is substituted with either an A, I, or L; wherein L1033 is substituted with either an A, I, or V; wherein D1034 is substituted with an E; wherein S1035 is substituted with either an A, G, M, or T; wherein T1036 is substituted with either an A, G, M, or S; wherein E1037 is substituted with a D; wherein A1038 is substituted with either a G, I, L, M, S, T, or V; wherein L1039 is substituted with either an A, I, or V; wherein C1040 is a C; wherein L1041 is substituted with either an A, I, or V; wherein A1042 is substituted with either a G, I, L, M, S, T, or V; wherein I1043 is substituted with either an A, V, or L; wherein S1044 is substituted with either an A, G, M, or T; wherein G1045 is substituted with either an A, M, S, or T; wherein N1046 is substituted with a Q; wherein S1047 is substituted with either an A, G, M, or T; wherein E1048 is substituted with a D; wherein Q1049 is substituted with a N; wherein M1050 is substituted with either an A, G, S, or T; wherein A1051 is substituted with either a G, I, L, M, S, T, or V; wherein S1052 is substituted with either an A, G, M, or T; wherein H1053 is substituted with either a K, or R; wherein S1054 is substituted with either an A, G, M, or T; wherein A1055 is substituted with either a G, I, L, M, S, T, or V; wherein V1056 is substituted with either an A, I, or L; wherein L1057 is substituted with either an A, I, or V; wherein E1058 is substituted with a D; wherein A1059 is substituted with either a G, I, L, M, S, T, or V; wherein G1060 is substituted with either an A, M, S, or T; wherein K1061 is substituted with either a R, or H; wherein N1062 is substituted with a Q; wherein L1063 is substituted with either an A, I, or V; wherein Y1064 is either an F, or W; wherein T1065 is substituted with either an A, G, M, or S; wherein F1066 is substituted with either a W, or Y; wherein C1067 is a C; wherein V1068 is substituted with either an A, I, or L; wherein S1069 is substituted with either an A, G, M, or T; wherein Y1070 is either an F, or W; wherein V1071 is substituted with either an A, I, or L; wherein D1072 is substituted with an E; wherein S1073 is substituted with either an A, G, M, or T; wherein I1074 is substituted with either an A, V, or L; wherein Q1075 is substituted with a N; wherein Q1076 is substituted with a N; wherein M1077 is substituted with either an A, G, S, or T; wherein R1078 is substituted with either a K, or H; wherein N1079 is substituted with a Q; wherein K1080 is substituted with either a R, or H; wherein F1081 is substituted with either a W, or Y; wherein A1082 is substituted with either a G, I, L, M, S, T, or V; wherein F1083 is substituted with either a W, or Y; wherein R1084 is substituted with either a K, or H; wherein E1085 is substituted with a D; wherein A1086 is substituted with either a G, I, L, M, S, T, or V; wherein I1087 is substituted with either an A, V, or L; wherein N1088 is substituted with a Q; wherein K1089 is substituted with either a R, or H; wherein L1090 is substituted with either an A, I, or V; wherein E1091 is substituted with a D; wherein N1092 is substituted with a Q; wherein N1093 is substituted with a Q; wherein L1094 is substituted with either an A, I, or V; wherein R1095 is substituted with either a K, or H; wherein E1096 is substituted with a D; wherein L1097 is substituted with either an A, I, or V; wherein Q1098 is substituted with a N; wherein I1099 is substituted with either an A, V, or L; wherein C1100 is a C; wherein P1101 is a P; wherein A1102 is substituted with either a G, I, L, M, S, T, or V; wherein S1103 is substituted with either an A, G, M, or T; wherein A1104 is substituted with either a G, I, L, M, S, T, or V; wherein G1105 is substituted with either an A, M, S, or T; wherein S1106 is substituted with either an A, G, M, or T; wherein G1107 is substituted with either an A, M, S, or T; wherein P1108 is a P; wherein A1109 is substituted with either a G, I, L, M, S, T, or V; wherein A1110 is substituted with either a G, I, L, M, S, T, or V; wherein T1111 is substituted with either an A, G, M, or S; wherein Q1112 is substituted with a N; wherein D1113 is substituted with an E; wherein F1114 is substituted with either a W, or Y; wherein S1115 is substituted with either an A, G, M, or T; wherein K1116 is substituted with either a R, or H; wherein L1117 is substituted with either an A, I, or V; wherein L1118 is substituted with either an A, I, or V; wherein S1119 is substituted with either an A, G, M, or T; wherein S1120 is substituted with either an A, G, M, or T; wherein V1121 is substituted with either an A, I, or L; wherein K1122 is substituted with either a R, or H; wherein E1123 is substituted with a D; wherein I1124 is substituted with either an A, V, or L; wherein S1125 is substituted with either an A, G, M, or T; wherein D1126 is substituted with an E; wherein I1127 is substituted with either an A, V, or L; wherein V1128 is substituted with either an A, I, or L; wherein Q1129 is substituted with a N; and/or wherein R1130 is substituted with either a K, or H of SEQ ID NO:2 in addition to any combination thereof. Other suitable substitutions within the BCR/ABL are encompassed by the present invention and are referenced elsewhere herein. The present invention also encompasses the use of these BCR/ABL conservative amino acid substituted polypeptides as immunogenic and/or antigenic epitopes as described elsewhere herein.

The present invention is directed to a mutant BCR/ABL sequence containing 1, 2, 3, 4, 5, 6, 7, 8, 9, 10, 11, 12, 13, 14, 15, 16, 17, 18, 19, 20 or more of the disclosed amino acid substitutions. The substitutions of the present invention also encompass additions and/or deletions of 1, 2, 3, 4, 5, 6, 7, 8, 9, 10, 11, 12, 13, 14, 15, 16, 17, 18, 19, 20 or more amino acids. Said mutants may preferably result in BCR-ABL kinase of the present invention being constitutively activated, and/or at least partially resistant to a protein tyrosine kinase inhibitor.

A further embodiment of the invention relates to a polypeptide which comprises the amino acid sequence of the present invention having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of the present invention, which contains at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), is 1-5, 5-10, 5-25, 5-50, 10-50 or 50-150, conservative amino acid substitutions are preferable.

The present invention provides isolated novel BCR-ABL polynucleotides encoding polypeptides comprising the amino acid sequence set forth in SEQ ID NO:2 or having substantial identity to the amino acid sequence set forth in SEQ ID NO:2 having at least a E507G mutation and one or more of the mutations described herein including any combination thereof.

Accordingly the present invention provides isolated novel BCR-ABL polypeptides comprising the amino acid sequence set forth in SEQ ID NO:2 or having substantial identity to the amino acid sequence set forth in SEQ ID NO:2 and having at least a E507G mutation and one or more of the mutations described herein including any combination thereof.

### Exemplary Indications, Conditions, Diseases, and Disorders

The present invention provides methods of determining responsiveness of an individual having a BCR-ABL associated disorder to a certain treatment regimen and methods of treating an individual having a BCR-ABL associated disorder.

The term “BCR-ABL” as used herein is inclusive of both wild-type and mutant BCR-ABL.

“BCR-ABL associated disorders” are those disorders which result from BCR-ABL activity, including mutant BCR-ABL activity, and/or which are alleviated by the inhibition of BCR-ABL, including mutant BCR-ABL, expression and/or activity. A reciprocal translocation between chromosomes 9 and 22 produces the oncogenic BCR-ABL fusion protein. The phrase “BCR-ABL associated disorders” is inclusive of “mutant BCR-ABL associated disorders”.

Disorders included in the scope of the present invention include, for example, leukemias, including, for example, chronic myeloid leukemia, acute lymphoblastic leukemia, and Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ALL, AML, imatinib-resistant CML, imatinib-intolerant CML, accelerated CML, lymphoid blast phase CML.

A “solid tumor” includes, for example, sarcoma, melanoma, carcinoma, prostate carcinoma, lung carcinoma, colon carcinoma, or other solid tumor cancer.

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ALL).

A “mutant BCR-ABL” encompasses a BCR-ABL tyrosine kinase with an amino acid sequence that differs from wild type BCR-ABL tyrosine kinase by one or more amino acid substitutions, additions or deletions. For example a substitution of the amino acid at position 507 of SEQ ID NO:2 with another amino acid would result in a mutant BCR-ABL tyrosine kinase.

“Mutant BCR-ABL associated disorder” is used to describe a BCR-ABL associated disorder in which the cells involved in said disorder are or become resistant to treatment with a kinase inhibitor used to treat said disorder as a result of a mutation in BCR-ABL. For example, a kinase inhibitor compound can be used to treat a cancerous condition, which compound inhibits the activity of wild type BCR-ABL which will inhibit proliferation and/or induce apoptosis of cancerous cells. Over time, a mutation can be introduced into the gene encoding BCR-ABL kinase, which can alter the amino acid sequence of the BCR-ABL kinase and cause the cancer cells to become resistant, or at least partially resistant, to treatment with the compound. Alternatively, a mutation can already be present within the gene encoding BCR-ABL kinase, either genetically or as a consequence of an oncogenic event, independent of treatment with a protein tyrosine kinase inhibitor, which can be one factor resulting in these cells propensity to differentiate into a cancerous or proliferative state, and also result in these cells being less sensitive to treatment with a protein tyrosine kinase inhibitor. Such situations are expected to result, either directly or indirectly, in a “mutant BCR-ABL kinase associated disorder” and treatment of such condition will require a compound that is at least partially effective against the mutant BCR-ABL, preferably against both wild type BCR-ABL and the mutant BCR-ABL. In the instance where an individual develops at least partial resistance to the kinase inhibitor imatinib, the mutant BCR-ABL associated disorder is one that results from an imatinib-resistant BCR-ABL mutation, or a protein tyrosine kinase inhibitor resistant BCR-ABL mutation. Similarly, in the instance where an individual develops at least partial resistance to the kinase inhibitor N-(2-chloro-6-methylphenyl)-2-[[6-[4-(2-hydroxyethyl)-1-piperazinyl]-2-methyl-4-pyrimidinyl]amino]-5-thiazolecarboxamide, the mutant BCR-ABL associated disorder is one that results from an N-(2-chloro-6-methylphenyl)-2-[[6-[4-(2-hydroxyethyl)-1-piperazinyl]-2-methyl-4-pyrimidinyl]amino]-5-thiazolecarboxamide resistant BCR-ABL mutation, or a protein tyrosine kinase inhibitor resistant BCR-ABL mutation. The present inventors discovered that after treatment with N-(2-chloro-6-methylphenyl)-2-[[6-[4-(2-hydroxyethyl)-1-piperazinyl]-2-methyl-4-pyrimidinyl]amino]-5-thiazolecarboxamide, certain individuals developed E507G mutations. The present invention provides, among other things, methods of treating mutant BCR-ABL associated disorders and methods of identifying if an individual has a mutant BCR-ABL associated disorder.

Protein tyrosine kinase-associated disorders of particular interest herein are those disorders which result, at least in part, from aberrant SRC or BCR-ABL (WT or mutant) activity and/or which are alleviated by the inhibition of SRC or BCR-ABL (WT or mutant) referred to herein as “SRC associated disorders”, “SRC associated cancer”, or “BCR-ABL associated disorders”, “BCR-ABL associated cancer” “SRC”, “SRC kinase”, and “Mutant SRC kinase” encompasses a SRC kinase with an amino acid sequence that differs from wild type SRC kinase by one or more amino acid substitutions, additions or deletions, and necessarily includes BCR-ABL encoding polynucleotides and polypeptides with one or more amino acid substitutions, additions, or deletions. Discussed herein are several mutant SRC kinases. SRC necessarily encompasses ABL, BCR/ABL, SRC including SRC family kinases such as c-Src, SRC/ABL, and other forms including, but not limited to, JAK, FAK, FPS, CSK, SYK, and BTK.

“Imatinib-resistant BCR-ABL mutation” refers to a specific mutation in the amino acid sequence of BCR-ABL that confers upon cells that express said mutation resistance to treatment with imatinib. As discussed herein such mutations can include mutations at the T315I position of BCR-ABL. Additional mutations that may render a BCR-ABL protein at least partially imatinib resistant can include, for example, E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, T315A, E316G, F317L, F317I, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, F486S, and E507G. Additional Imatinib-resistant BCR-ABL mutations may also include other BCR-ABL mutations disclosed elsewhere herein.

“Dasatinib-resistant BCR-ABL mutation” refers to a specific mutation in the amino acid sequence of BCR-ABL that confers upon cells that express said mutation at least partial resistance to treatment with dasatinib. As discussed herein such mutations can include mutations at the T315I, T315A, F317A, F317I, and E507G position of BCR-ABL. Additional dasatinib-resistant BCR-ABL mutations may also include other BCR-ABL mutations disclosed elsewhere herein.

“N-(2-chloro-6-methylphenyl)-2-[[6-[4-(2-hydroxyethyl)-1-piperazinyl]-2-methyl-4-pyrimidinyl]amino]-5-thiazolecarboxamide-resistant BCR-ABL mutation” refers to a specific mutation in the amino acid sequence of BCR-ABL that confers upon cells that express said mutation at least partial resistance to treatment with N-(2-chloro-6-methylphenyl)-2-[[6-[4-(2-hydroxyethyl)-1-piperazinyl]-2-methyl-4-pyrimidinyl]amino]-5-thiazolecarboxamide. As discussed herein such mutations can include the E507G mutation. Additional mutations that render a BCR-ABL protein at least partially N-(2-chloro-6-methylphenyl)-2-[[6-[4-(2-hydroxyethyl)-1-piperazinyl]-2-methyl-4-pyrimidinyl]amino]-5-thiazolecarboxamide resistant include, for example, T315I, T315A, F317I, and F317L.

“Imatinib-resistant CML” refers to a CML in which the cells involved in CML are resistant to treatment with imatinib. Generally it is a result of a mutation in BCR-ABL.

“Imatinib-intolerant CML” refers to a CML in which the individual having the CML is intolerant to treatment with imatinib, i.e., the toxic and/or detrimental side effects of imatinib outweigh any therapeutically beneficial effects.

The invention provides a method of treating cancers, including both primary and metastatic cancers, including solid tumors such as those of the breast, colon, and prostate, as well as lymphomas and leukemias (including CML, AML and ALL), cancers of endothelial tissues, and including cancers which are resistant to other therapies, including other therapies involving administration of kinase inhibitors such as imatinib. Specifically, and without limitation, the invention provides the use of a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, for treating disorders, for example cancers, which are resistant to other therapies involving administration of kinase inhibitors such as imatinib.

### Detection Methods

The invention provides methods of screening a biological sample from an individual for the presence of at least one mutation in the BCR-ABL kinase sequence, as well as methods for identifying a cell that expresses mutant BCR-ABL kinase.

Methods of identifying the amino acid and nucleic acid sequence of a wild-type or mutant BCR-ABL polynucleotide or BCR-ABL polypeptide are known in the art. Standard molecular biology techniques are contemplated for precisely determining a BCR-ABL mutation in the cells of a given individual.

Antibodies that immunospecifically bind to a mutant BCR-ABL kinase can be used in identifying one or more of the BCR-ABL mutants described herein. Contemplated herein are antibodies that specifically bind to a mutant BCR-ABL kinase of the present invention and that do not bind (or bind weakly) to wild type BCR-ABL protein or polypeptides. Anti-mutant BCR-ABL kinase antibodies include, for example, monoclonal and polyclonal antibodies as well as fragments containing the antigen binding domain and/or one or more complementarity determining regions of these antibodies.

For some applications, it may be desirable to generate antibodies which specifically react with a particular mutant BCR-ABL kinase protein and/or an epitope within a particular structural domain. For example, antibodies useful for diagnostic purposes can be those which react with an epitope in a mutated region of the BCR-ABL protein as expressed in cancer cells. For example, antibodies that bind specifically to a E507G mutant BCR-ABL kinase. Such antibodies can be generated by using the mutant BCR-ABL kinase protein described herein, or using peptides derived from various domains thereof, as an immunogen.

Mutant BCR-ABL kinase antibodies of the invention can be particularly useful in cancer (e.g., chronic myeloid leukemia, acute lymphoblastic leukemia, Philadelphia chromosome positive acute lymphoblastic leukemia (Ph+ALL, GIST)) therapeutic strategies, diagnostic and prognostic assays, and imaging methodologies. Similarly, such antibodies can be useful in the diagnosis, and/or prognosis of other cancers, to the extent such mutant BCR-ABL kinase is also expressed or overexpressed in other types of cancer. The invention provides various immunological assays useful for the detection and quantification of mutant BCR-ABL kinase proteins and polypeptides. Such assays generally comprise one or more mutant BCR-ABL kinase antibodies capable of recognizing and binding a mutant BCR-ABL kinase protein, as appropriate, and can be performed within various immunological assay formats well known in the art, including, for example, various types of radioimmunoassays, enzyme-linked immunosorbent assays (ELISA), enzyme-linked immunofluorescent assays (ELIFA), and the like. In addition, immunological imaging methods capable of detecting cancer cells are also provided by the invention including, for example, imaging methods using labeled mutant BCR-ABL kinase antibodies. Such assays can be used clinically in the detection, monitoring, and prognosis of cancers.

Accordingly, the present invention provides methods of assaying for the presence of a mutant BCR-ABL polypeptide of the present invention. By way of example only, in certain embodiments, an antibody raised against the fragment, or other binding moiety capable of specifically binding to the target analyte, is immobilised onto a solid substrate to form a first complex and a biological test sample from a patient is brought into contact with the bound molecule. After a suitable period of incubation, for a period of time sufficient to allow formation of an antibody-secondary complex, a second antibody labelled with a reporter molecule capable of producing a detectable signal is then added and incubated, allowing sufficient time for the formation of a tertiary complex. Any unreacted material is washed away, and the presence of the tertiary complex is determined by observation of a signal produced by the reporter molecule. The results may either be qualitative, by simple observation of the visible signal or may be quantitated by comparison with a control sample containing known amounts of hapten. Variations of this assay include a simultaneous assay, in which both sample and labelled antibody are added simultaneously to the bound antibody, or a reverse assay in which the labelled antibody and sample to be tested are first combined, incubated and then added simultaneously to the bound antibody. These techniques are well known to those skilled in the art, and the possibility of variations will be readily apparent.

By “reporter molecule”, as used in the present specification, is meant a molecule which, by its chemical nature, produces an analytically identifiable signal which allows the detection of antigen-bound antibody. Detection may be either qualitative or quantitative. The most commonly used reporter molecule in this type of assay are either enzymes, fluorophores or radionuclide containing molecules (i.e. radioisotopes).

The solid substrate is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing the molecule to the insoluble carrier.

The expression profiles of mutant BCR-ABL kinases can be used as diagnostic markers for disease states. The status of mutant BCR-ABL kinase gene products in patient samples can be analyzed by a variety protocols that are well known in the art including the following non-limiting types of assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, microfluidic genotype assays, immunohistochemical analysis, the variety of Northern blotting techniques including in situ hybridization, RT-PCR analysis (for example on laser capture micro-dissected samples), western blot analysis, tissue array analysis, and any other methods known in the art or described elsewhere herein.

Specifically encompassed by the present invention are the following, non-limiting genotyping methods: Landegren, U., Nilsson, M. & Kwok, P. Genome Res 8, 769-776 (1998); Kwok, P., Pharmacogenomics 1, 95-100 (2000); Gut, I., Hum Mutat 17, 475-492 (2001); Whitcombe, D., Newton, C. & Little, S., Curr Opin Biotechnol 9, 602-608 (1998); Tillib, S. & Mirzabekov, A., Curr Opin Biotechnol 12, 53-58 (2001); Winzeler, E. et al., Science 281, 1194-1197 (1998); Lyamichev, V. et al., Nat Biotechnol 17, 292-296 (1999); Hall, J. et al., Proc Natl Acad Sci USA 97, 8272-8277 (2000); Mein, C. et al., Genome Res 10, 333-343 (2000); Ohnishi, Y. et al., J Hum Genet 46, 471-477 (2001); Nilsson, M. et al., Science 265, 2085-2088 (1994); Baner, J., Nilsson, M., Mendel-Hartvig, M. & Landegren, U., Nucleic Acids Res 26, 5073-5078 (1998); Baner, J. et al., Curr Opin Biotechnol 12, 11-15 (2001); Hatch, A., Sano, T., Misasi, J. & Smith, C., Genet Anal 15, 35-40 (1999); Lizardi, P. et al., Nat Genet 19, 225-232 (1998); Zhong, X., Lizardi, P., Huang, X., Bray-Ward, P. & Ward, D., Proc Natl Acad Sci USA 98, 3940-3945 (2001); Faruqi, F. et al. BMC Genomics 2, 4 (2001); Livak, K., Gnet Anal 14, 143-149 (1999); Marras, S., Kramer, F. & Tyagi, S., Genet Anal 14, 151-156 (1999); Ranade, K. et al., Genome Res 11, 1262-1268 (2001); Myakishev, M., Khripin, Y., Hu, S. & Hamer, D., Genome Re 11, 163-169 (2001); Beaudet, L., Bedard, J., Breton, B., Mercuri, R. & Budarf, M., Genome Res 11, 600-608 (2001); Chen, X., Levine, L. & PY, K., Genome Res 9, 492-498 (1999); Gibson, N. et al., Clin Chem 43, 1336-1341 (1997); Latif, S., Bauer-Sardina, I., Ranade, K., Livak, K. & PY, K., Genome Res 11, 436-440 (2001); Hsu, T., Law, S., Duan, S., Neri, B. & Kwok, P., Clin Chem 47, 1373-1377 (2001); Alderborn, A., Kristofferson, A. & Hammerling, U., Genome Res 10, 1249-1258 (2000); Ronaghi, M., Uhlen, M. & Nyren, P., Science 281, 363, 365 (1998); Ronaghi, M., Genome Res 11, 3-11 (2001); Pease, A. et al., Proc Natl Acad Sci USA 91, 5022-5026 (1994); Southern, E., Maskos, U. & Elder, J., Genomics 13, 1008-1017 (1993); Wang, D. et al., Science 280, 1077-1082 (1998); Brown, P. & Botstein, D., Nat Genet 21, 33-37 (1999); Cargill, M. et al. Nat Genet 22, 231-238 (1999); Dong, S. et al., Genome Res 11, 1418-1424 (2001); Halushka, M. et al., Nat Genet 22, 239-247 (1999); Hacia, J., Nat Genet 21, 42-47 (1999); Lipshutz, R., Fodor, S., Gingeras, T. & Lockhart, D., Nat Genet 21, 20-24 (1999); Sapolsky, R. et al., Genet Anal 14, 187-192 (1999); Tsuchihashi, Z. & Brown, P., J Virol 68, 5863 (1994); Herschlag, D., J Biol Chem 270, 20871-20874 (1995); Head, S. et al., Nucleic Acids Res 25, 5065-5071 (1997); Nikiforov, T. et al., Nucleic Acids Res 22, 4167-4175 (1994); Syvanen, A. et al., Genomics 12, 590-595 (1992); Shumaker, J., Metspalu, A. & Caskey, C., Hum Mutat 7, 346-354 (1996); Lindroos, K., Liljedahl, U., Raitio, M. & Syvanen, A., Nucleic Acids Res 29, E69-9 (2001); Lindblad-Toh, K. et al., Nat Genet 24, 381-386 (2000); Pastinen, T. et al., Genome Res 10, 1031-1042 (2000); Fan, J. et al., Genome Res 10, 853-860 (2000); Hirschhorn, J. et al., Proc Natl Acad Sci USA 97, 12164-12169 (2000); Bouchie, A., Nat Biotechnol 19, 704 (2001); Hensel, M. et al., Science 269, 400-403 (1995); Shoemaker, D., Lashkari, D., Morris, D., Mittmann, M. & Davis, R. Nat Genet 14, 450-456 (1996); Gerry, N. et al., J Mol Biol 292, 251-262 (1999); Ladner, D. et al., Lab Invest 81, 1079-1086 (2001); Jannone, M. et al. Cytometry 39, 131-140 (2000); Fulton, R., McDade, R., Smith, P., Kienker, L. & Kettman, J. J., Clin Chem 43, 1749-1756 (1997); Armstrong, B., Stewart, M. & Mazumder, A., Cytometry 40, 102-108 (2000); Cai, H. et al., Genomics 69, 395 (2000); Chen, J. et al., Genome Res 10, 549-557 (2000); Ye, F. et al. Hum Mutat 17, 305-316 (2001); Michael, K., Taylor, L., Schultz, S. & Walt, D., Anal Chem 70, 1242-1248 (1998); Steemers, F., Ferguson, J. & Walt, D., Nat Biotechnol 18, 91-94 (2000); Chan, W. & Nie, S., Science 281, 2016-2018 (1998); Han, M., Gao, X., Su, J. & Nie, S., Nat Biotechnol 19, 631-635 (2001); Griffin, T. & Smith, L., Trends Biotechnol 18, 77-84 (2000); Jackson, P., Scholl, P. & Groopman, J., Mol Med Today 6, 271-276 (2000); Haff, L. & Smirnov, I., Genome Res 7, 378-388 (1997); Ross, P., Hall, L., Smirnov, I. & Haff, L., Nat Biotechnol 16, 1347-1351 (1998); Bray, M., Boerwinkle, E. & Doris, P. Hum Mutat 17, 296-304 (2001); Sauer, S. et al., Nucleic Acids Res 28, E13 (2000); Sauer, S. et al., Nucleic Acids Res 28, E100 (2000); Sun, X., Ding, H., Hung, K. & Guo, B., Nucleic Acids Res 28, E68 (2000); Tang, K. et al., Proc Natl Acad Sci USA 91, 10016-10020 (1999); L1, J. et al., Electrophoresis 20, 1258-1265 (1999); Little, D., Braun, A., O'Donnell, M. & Koster, H., Nat Med 3, 1413-1416 (1997); Little, D. et al. Anal Chem 69, 4540-4546 (1997); Griffin, T., Tang, W. & Smith, L., Nat Biotechnol 15, 1368-1372 (1997); Ross, P., Lee, K. & Belgrader, P., Anal Chem 69, 4197-4202 (1997); Jiang-Baucom, P., Girard, J., Butler, J. & Belgrader, P., Anal Chem 69, 4894-4898 (1997); Griffin, T., Hall, J., Prudent, J. & Smith, L., Proc Natl Acad Sci USA 96, 6301-6306 (1999); Kokoris, M. et al., Mol Diagn 5, 329-340 (2000); Jurinke, C., van den Boom, D., Cantor, C. & Koster, H. (2001); and/or Taranenko, N. et al., Genet Anal 13, 87-94 (1996), all of which are incorporated herein by reference in their entirety.

The following additional genotyping methods are also encompassed by the present invention: the methods described and/or claimed in U.S. Pat. No. 6,458,540, incorporated herein by reference in its entirety; and the methods described and/or claimed in U.S. Pat. No. 6,440,707, incorporated herein by reference in its entirety.

Probes and primers can be designed so as to be specific to such mutation analysis and can be derived from the wild type BCR-ABL sequence, segments and complementary sequences thereof.

Additionally, the invention provides assays for the detection of mutant BCR-ABL kinase polynucleotides in a biological sample, such as cell preparations, and the like. A number of methods for amplifying and/or detecting the presence of mutant BCR-ABL kinase polynucleotides are well known in the art and can be employed in the practice of this aspect of the invention.

In certain embodiments, a method for detecting a mutant BCR-ABL kinase mRNA in a biological sample comprises producing cDNA from the sample by reverse transcription using at least one primer; amplifying the cDNA so produced using mutant BCR-ABL kinase polynucleotides as sense and antisense primers to amplify mutants BCR-ABL kinase cDNAs therein; and detecting the presence of the amplified mutant BCR-ABL kinase cDNA. Any number of appropriate sense and antisense probe combinations can be designed from the nucleotide sequences provided for a mutant BCR-ABL kinase and used for this purpose.

The invention also provides assays for detecting the presence of a mutant BCR-ABL kinase protein in a biological sample. Methods for detecting a mutant BCR-ABL kinase protein are also well known and include, for example, immunoprecipitation, immunohistochemical analysis, Western Blot analysis, molecular binding assays, ELISA, ELIFA and the like. For example, in one embodiment, a method of detecting the presence of a mutant BCR-ABL kinase protein in a biological sample comprises first contacting the sample with a BCR-ABL antibody, a mutant BCR-ABL kinase-reactive fragment thereof, or a recombinant protein containing an antigen binding region of a mutant BCR-ABL kinase antibody; and then detecting the binding of mutant BCR-ABL kinase protein in the sample thereto.

Methods for identifying a cell that expresses mutant BCR-ABL kinase are also provided. In one embodiment, an assay for identifying a cell that expresses a mutant BCR-ABL kinase gene comprises detecting the presence of mutant BCR-ABL mRNA in the cell. Methods for the detection of particular mRNAs in cells are well known and include, for example, hybridization assays using complementary DNA probes (such as in situ hybridization using labeled mutant BCR-ABL kinase riboprobes, Northern blot and related techniques) and various nucleic acid amplification assays (such as RT-PCR using complementary primers specific for a mutant BCR-ABL kinase, and other amplification type detection methods, such as, for example, branched DNA, SISBA, TMA and the like).

The detection methods of the present invention also include methods for identifying amino acid positions within the BCR-ABL polypeptide that may confer at least partial resistance to a tyrosine kinase inhibitor. The methods can comprise the steps of creating a co-crystal of the polypeptide with the BCR-ABL inhibitor, and identifying the amino acid positions of the polypeptide that either contact, bond, interface, or interact with the BCR-ABL inhibitor, or identifying those amino acid positions that stabilize the amino acid positions of the polypeptide that either contact, bond, interface, or interact with the BCR-ABL inhibitor. Methods of creating crystal structures are known in the art and can include, for example, the use of X-ray crystallography to determine the crystal structure (See, for example, Tokarski et al., Cancer Res (2006), 66(11), 5790-5797) In certain embodiments, the contact, bond, interface, or interact amino acids will be at positions 248, 299, 315, and/or 317. In certain embodiments, the contact, bond, interface, or interact amino acids, or the amino acids that stabilize the amino acids that either contact, bond, interface, or interact with the BCR-ABL inhibitor, will be at positions 244, 248, 255, 290, 299, 313, 315, 316, 317, 318, 320, 321 and/or 380.

### Treatment Regimens

The invention encompasses treatment methods based upon the demonstration that patients harboring different BCR-ABL mutations have varying degrees of resistance and/or sensitivity to imatinib and/or N-(2-chloro-6-methylphenyl)-2-[[6-[4-(2-hydroxyethyl)-1-piperazinyl]-2-methyl-4-pyrimidinyl]amino]-5-thiazolecarboxamide, respectively. Thus the methods of the present invention can be used, for example, in determining whether or not to treat an individual with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, whether or not to treat an individual with a more aggressive dosage regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, or whether or not to treat an individual with combination therapy, i.e., a combination of tyrosine kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and additional BCR-ABL inhibitors(s) (e.g., such as imatinib, AMN107, PD180970, GGP76030, AP23464, SKI 606, NS-187, and/or AZD0530); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a tubulin stabilizing agent (such as, for example, pacitaxol, epothilone, taxane, and the like.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and ATP non-competitive inhibitors ONO12380; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and Aurora kinase inhibitor VX-680; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and p38 MAP kinase inhibitor BIRB-796; any other combination disclosed herein.

The terms “treating”, “treatment” and “therapy” as used herein refer to curative therapy, prophylactic therapy, preventative therapy, and mitigating disease therapy.

In certain embodiments, the present invention provides a method of identifying a mutation in a BCR-ABL polynucleotide in a mammalian cell, wherein the mutation in a BCR-ABL polynucleotide is associated with resistance to inhibition of BCR-ABL kinase activity by N-(2-chloro-6-methylphenyl)-2-[[6-[4-(2-hydroxyethyl)-1-piperazinyl]-2-methyl-4-pyrimidinyl]amino]-5-thiazolecarboxamide, the method comprising determining the sequence of at least one BCR-ABL kinase polynucleotide expressed by the mammalian cell and comparing the sequence of the BCR-ABL kinase polynucleotide to the wild type BCR-ABL kinase polynucleotide sequence. As described herein the polynucleotide identified can encode a polypeptide having at least one amino acid difference from the wild type BCR-ABL kinase amino acid sequence, i.e., a E507G mutation or other BCR-ABL mutation disclosed herein including any combination thereof.

In the method disclosed above, the mammalian cell can be a human cancer cell. The human cancer cell can be one obtained from an individual treated having a BCR-ABL associated disorder.

For use herein, a BCR-ABL inhibitor refers to any molecule or compound that can partially inhibit BCR-ABL or mutant BCR-ABL activity or expression. These include inhibitors of the Src family kinases such as BCR/ABL, ABL, c-Src, SRC/ABL, and other forms including, but not limited to, JAK, FAK, FPS, CSK, SYK, and BTK. A series of inhibitors, based on the 2-phenylaminopyrimidine class of pharmacophotes, has been identified that have exceptionally high affinity and specificity for Abl (see, e.g., Zimmerman et al., Bioorg, Med. Chem. Lett. 7, 187 (1997)). All of these inhibitors are encompassed within the term a BCR-ABL inhibitor. Imatinib, one of these inhibitors, also known as STI-571 (formerly referred to as Novartis test compound CGP 57148 and also known as Gleevec), has been successfully tested in clinical trail a therapeutic agent for CML. AMN107, is another BCR-ABL kinase inhibitor that was designed to fit into the ATP-binding site of the BCR-ABL protein with higher affinity than imatinib. In addition to being more potent than imatinib (IC50<30 nM) against wild-type BCR-ABL, AMN107 is also significantly active against 32/33 imatinib-resistant BCR-ABL mutants. In preclinical studies, AMN107 demonstrated activity in vitro and in vivo against wild-type and imatinib-resistant BCR-ABL-expressing cells. In phase I/II clinical trials, AMN107 has produced haematological and cytogenetic responses in CML patients, who either did not initially respond to imatinib or developed imatinib resistance (Weisberg et al., British Journal of Cancer (2006) 94, 1765-1769, incorporated herein by reference in its entirety and for all purposes). SKI-606, NS-187, AZD0530, PD180970, CGP76030, and AP23464 are all examples of kinase inhibitors that can be used in the present invention. SKI-606 is a 4-anilino-3-quinolinecarbonitrile inhibitor of Abl that has demonstrated potent antiproliferative activity against CML cell (Golas et al., Cancer Research (2003) 63, 375-381). AZD0530 is a dual Abl/Src kinase inhibitor that is in ongoing clinical trials for the treatment of solid tumors and leukemia (Green et al., Preclinical Activity of AZD0530, a novel, oral, potent, and selective inhibitor of the Src family kinases. Poster 3161 presented at the EORTC-NCI-AACR, Geneva Switzerland 28 Sep. 2004). PD180970 is a pyrido[2,3-d]pyrimidine derivative that has been shown to inhibit BCR-ABL and induce apoptosis in BCR-ABL expressing leukemic cells (Rosee et al., Cancer Research (2002) 62, 7149-7153). CGP76030 is dual-specific Src and Abl kinase inhibitor shown to inhibit the growth and survival of cells expressing imatinib-resistant BCR-ABL kinases (Warmuth et al., Blood, (2003) 101(2), 664-672). AP23464 is an ATP-based kinase inhibitor that has been shown to inhibit imatinib-resistant BCR-ABL mutants (O'Hare et al., Clin Cancer Res (2005) 11(19), 6987-6993). NS-187 is a selective dual Bcr-Abl/Lyn tyrosine kinase inhibitor that has been shown to inhibit imatinib-resistant BCR-ABL mutants (Kimura et al., Blood, 106(12):3948-3954 (2005)).

A “farnysyl transferase inhibitor” can be any compound or molecule that inhibits farnysyl transferase. The farnysyl transferase inhibitor can have formula (II), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt. The compound of formula (II) is a cytotoxic FT inhibitor which is known to kill non-proliferating cancer cells preferentially. The compound of formula (II) can further be useful in killing stem cells.

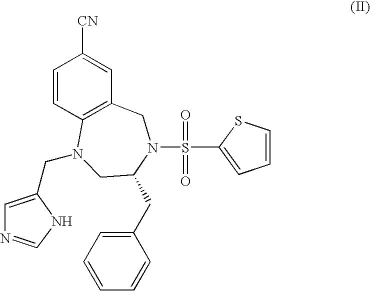

The compound of formula (II), its preparation, and uses thereof are described in U.S. Pat. No. 6,011,029, which is herein incorporated by reference in its entirety and for all purposes. Uses of the compound of formula (II) are also described in WO2004/015130, published Feb. 19, 2004, which is herein incorporated by reference in its entirety and for all purposes.

For use herein, combination therapy refers to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof with a second therapy at such time that both the second therapy and N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, will have a therapeutic effect. Such administration can involve concurrent (i.e., at the same time), prior, or subsequent administration of the second therapy with respect to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof.

Treatment regimens can be established based upon the presence of one or more mutant BCR-ABL kinases disclosed herein. For example, the invention encompasses screening cells from an individual who may suffer from, or is suffering from, a disorder that is commonly treated with a kinase inhibitor. Such a disorder can include myeloid leukemia or disorders associated therewith, or cancers described herein. The cells of an individual are screened, using methods known in the art, for identification of a mutation in a BCR-ABL kinase. Mutations of interest are those that result in BCR-ABL kinase being constitutively activated. Specific mutations include, for example, E507G (wherein the glutamic acid at position 507 is replaced with a glycine). Other mutations include, for example, E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, T315A, E316G, F317L, F317I, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, F486S or any combination thereof, i.e., M244V, G250E, Q252H, Q252R, Y253F, Y253H, E255K, E255V, T315I, T315A, F317L, F317I, M351T, E355G, F359V, H396R, F486S and any combination thereof, M244V, E279K, F359C, F359I, L364I, L387M, F486S and any combination thereof, and L248R, Q252H, E255K, V299L, T315I, T315A, F317V, F317I, F317L, F317S and any combination thereof. Additional BCR-ABL mutations are also contemplated by the present invention and are disclosed herein.

If an activating BCR-ABL kinase mutation is found in the cells from said individual, treatment regimens can be developed appropriately. For example, an identified mutation can indicate that said cells are or will become at least partially resistant to commonly used kinase inhibitors. For example, a E507G mutation can indicate that the cells in an individual either are or are expected to become at least partially resistant to treatment with a kinase inhibitor such as N-(2-chloro-6-methylphenyl)-2-[[6-[4-(2-hydroxyethyl)-1-piperazinyl]-2-methyl-4-pyrimidinyl]amino]-5-thiazolecarboxamide. As disclosed herein, in such cases, treatment can include the use of an increased dosing frequency or increased dosage of N-(2-chloro-6-methylphenyl)-2-[[6-[4-(2-hydroxyethyl)-1-piperazinyl]-2-methyl-4-pyrimidinyl]amino]-5-thiazolecarboxamide or a salt, hydrate, or solvate thereof, a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and another kinase inhibitor drug such as imatinib, AMN107, PD180970, GGP76030, AP23464, SKI 606, and/or AZD0530; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; any other combination disclosed herein; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein. In one aspect, an increased level of N-(2-chloro-6-methylphenyl)-2-[[6-[4-(2-hydroxyethyl)-1-piperazinyl]-2-methyl-4-pyrimidinyl]amino]-5-thiazolecarboxamide would be about 10, 20, 30, 40, 50, 60, 70, 80, 90, or 95% more than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual, or about 1.5×, 2×, 2.5×, 3×, 3.5×, 4×, 4.5×, 5×, 6×, 7×, 8×, 9×, or 10× more N-(2-chloro-6-methylphenyl)-2-[[6-[4-(2-hydroxyethyl)-1-piperazinyl]-2-methyl-4-pyrimidinyl]amino]-5-thiazolecarboxamide than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual.

Additionally, dosage regimens can be further adapted based upon the presence of additional one or more amino acid mutation(s) in a BCR-ABL kinase. As described herein, a mutation in E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, T315A, E316G, F317L, F317I, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, F486S, or any combination thereof can indicate that the BCR-ABL kinase has developed at least partial resistance to therapy with a protein kinase inhibitor such as imitinab. Additional BCR-ABL mutations are also contemplated by the present invention and are disclosed herein.

A therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof can be orally administered as an acid salt of N-(2-chloro-6-methylphenyl)-2-[[6-[4-(2-hydroxyethyl)-1-piperazinyl]-2-methyl-4-pyrimidinyl]amino]-5-thiazolecarboxamide. The actual dosage employed can be varied depending upon the requirements of the patient and the severity of the condition being treated. Determination of the proper dosage for a particular situation is within the skill of the art. The effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof (and Compound I salt) can be determined by one of ordinary skill in the art, and includes exemplary dosage amounts for an adult human of from about 0.05 to about 100 mg/kg of body weight of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, per day, which can be administered in a single dose or in the form of individual divided doses, such as from 1, 2, 3, or 4 times per day. In certain embodiments, N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof is administered 2 times per day at 70 mg. Alternatively, it can be dosed at, for example, 50, 70, 90, 100, 110, or 120 BID, or 100, 140, or 180 once daily. It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders. The same also applies to Compound II or any combination of Compound I and II, or any combination disclosed herein.

A method of determining the responsiveness of an individual suffering from a protein tyrosine kinase-associated disorder to a combination of kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, is disclosed herein. For example, an individual can be determined to be a positive responder (or cells from said individual would be expected to have a degree of sensitivity) to a certain kinase inhibitor based upon the presence of a mutant BCR-ABL kinase. As disclosed herein, cells that exhibit certain mutations at amino acid positions 507 of BCR-ABL kinase of SEQ ID NO:2, or other BCR-ABL mutations disclosed herein, can develop at least partial resistance to of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof. Therefore, individuals suffering from a protein tyrosine kinase-associated disorder whose cells exhibit such a mutation are or would be expected to be partially negative responders to a particular treatment regimen with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof but a positive responder to a more aggressive treatment regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof or to combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and imatinib or other therapy.

A treatment regimen is a course of therapy administered to an individual suffering from a protein kinase associated disorder that can include treatment with one or more kinase inhibitors, as well as other therapies such as radiation and/or other agents (i.e., combination therapy). When more than one therapy is administered, the therapies can be administered concurrently or consecutively (for example, more than one kinase inhibitor can be administered together or at different times, on a different schedule). Administration of more than one therapy can be at different times (i.e., consecutively) and still be part of the same treatment regimen. As disclosed herein, for example, cells from an individual suffering from a protein kinase associated disorder can be found to develop at least partial resistance to N-(2-chloro-6-methylphenyl)-2-[[6-[4-(2-hydroxyethyl)-1-piperazinyl]-2-methyl-4-pyrimidinyl]amino]-5-thiazolecarboxamide. Based upon the present discovery that such cells can be sensitive to combination therapy or a more aggressive dosage or dosing regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, a treatment regimen can be established that includes treatment with the combination either as a monotherapy, or in combination with another kinase inhibitor, or in combination with another agent as disclosed herein. Additionally, the combination can be administered with radiation or other known treatments.

Therefore the present invention includes a method for establishing a treatment regimen for an individual suffering from a protein tyrosine kinase associated disorder or treating an individual suffering from a protein tyrosine kinase disorder comprising determining whether a biological sample obtained from an individual has at least a E507G mutation in the BCR-ABL kinase, or other BCR-ABL mutation disclosed herein, and administering to the subject an appropriate treatment regimen based on whether the mutation is present. The determination can be made by any method known in the art, for example, by screening said sample of cells for the presence of at least one mutation in a BCR-ABL kinase sequence or by obtaining information from a secondary source that the individual has the specified BCR-ABL kinase mutation.

In practicing the many aspects of the invention herein, biological samples can be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

Pharmaceutical compositions for use in the present invention can include compositions comprising one or a combination of inhibitors of a mutant BCR-ABL kinase in an effective amount to achieve the intended purpose. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity can be determined by standard pharmaceutical procedures in cell cultures or experimental animals, for example the ED50 (the dose therapeutically effective in 50% of the population) and LD50 (the dose lethal to 50% of the population).

Dosage regimens involving N-(2-chloro-6-methylphenyl)-2-[[6-[4-(2-hydroxyethyl)-1-piperazinyl]-2-methyl-4-pyrimidinyl]amino]-5-thiazolecarboxamide useful in practicing the present invention are described in U.S. Ser. No. 10/395,503, filed Mar. 24, 2003; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study”, by Moshe Talpaz, et al.; which are hereby incorporated herein by reference in their entirety and for all purposes.

A “therapeutically effective amount” of an inhibitor of a mutant BCR-ABL kinase can be a function of the mutation present. For example Shah et al disclose that cell lines with certain mutations in BCR-ABL kinase are more sensitive to N-(2-chloro-6-methylphenyl)-2-[[6-[4-(2-hydroxyethyl)-1-piperazinyl]-2-methyl-4-pyrimidinyl]amino]-5-thiazolecarboxamide than cell lines with different BCR-ABL kinase mutations. For example, cells comprising a F317L mutation in BCR-ABL kinase may require three to five-fold higher concentration of N-(2-chloro-6-methylphenyl)-2-[[6-[4-(2-hydroxyethyl)-1-piperazinyl]-2-methyl-4-pyrimidinyl]amino]-5-thiazolecarboxamide than cell lines expressing a F317I mutation. One skilled in the art will appreciate the difference in sensitivity of the mutant BCR-ABL kinase cells and determine a therapeutically effective dose accordingly.

Examples of predicted therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide that may be warranted based upon the relative sensitivity of BCR-ABL kinase mutants to N-(2-chloro-6-methylphenyl)-2-[[6-[4-(2-hydroxyethyl)-1-piperazinyl]-2-methyl-4-pyrimidinyl]amino]-5-thiazolecarboxamide compared to wild-type BCR-ABL kinase can be determined using various in vitro biochemical assays including cellular proliferation, BCR-ABL tyrosine phosphorylation, peptide substrate phosphorylation, and/or autophosphorylation assays. For example, approximate therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide can be calculated based upon multiplying the typical dose with the fold change in sensitivity in anyone or more of these assays for each BCR-ABL kinase mutant. O'Hare et al. (Cancer Research, 65(11):4500-5 (2005), which is hereby incorporated by reference in its entirety and for all purposes) performed analysis of the relative sensitivity of N-(2-chloro-6-methylphenyl)-2-[[6-[4-(2-hydroxyethyl)-1-piperazinyl]-2-methyl-4-pyrimidinyl]amino]-5-thiazolecarboxamide with several clinically relevant BCR-ABL Kinase mutants. For example, the E255V mutant had a fold change of “1” in the GST-Abl kinase assay, whereas this same mutant had a fold change of “14” in the cellular proliferation assay. Thus, a therapeutically relevant dose of N-(2-chloro-6-methylphenyl)-2-[[6-[4-(2-hydroxyethyl)-1-piperazinyl]-2-methyl-4-pyrimidinyl]amino]-5-thiazolecarboxamide for patients harboring this mutation could range, for example, anywhere from 1 to 14 fold higher than the typical dose. Accordingly, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide for any of the BCR-ABL kinase mutants can be, for example, 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 folder higher than the prescribed dose. Alternatively, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide can be, for example, 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01× of the prescribed dose.

According to O'hare et al., the M244V mutant had a fold change of “1.3” in the GST-Abl kinase assay, a fold change of “1.1” in the autophosphorylation assay, and a fold change of “2” in the cellular proliferation assay; the G250E mutant had a fold change of “0.5” in the GST-Abl kinase assay, a fold change of “3” in the autophosphorylation assay, and a fold change of “2” in the cellular proliferation assay; the Q252H mutant had a fold change of “4” in the cellular proliferation assay; the Y253F mutant had a fold change of “0.6” in the GST-Abl kinase assay, a fold change of “4” in the autophosphorylation assay, and a fold change of “4” in the cellular proliferation assay; the Y253H mutant had a fold change of “3” in the GST-Abl kinase assay, a fold change of “2” in the autophosphorylation assay, and a fold change of “2” in the cellular proliferation assay; the E255K mutant had a fold change of “0.3” in the GST-Abl kinase assay, a fold change of “2” in the autophosphorylation assay, and a fold change of “7” in the cellular proliferation assay; the F317L mutant had a fold change of “1.5” in the GST-Abl kinase assay, a fold change of “1.4” in the autophosphorylation assay, and a fold change of “9” in the cellular proliferation assay; the M351T mutant had a fold change of “0.2” in the GST-Abl kinase assay, a fold change of “2” in the autophosphorylation assay, and a fold change of “1.4” in the cellular proliferation assay; the F359V mutant had a fold change of “0.8” in the GST-Abl kinase assay, a fold change of “2” in the autophosphorylation assay, and a fold change of “3” in the cellular proliferation assay; the H396R mutant had a fold change of “1.3” in the GST-Abl kinase assay, a fold change of “3” in the autophosphorylation assay, and a fold change of “2” in the cellular proliferation assay.

For patients harboring the T315I mutation, either alone or in combination with another BCR-ABL mutation disclosed herein, administration of higher doses of N-(2-chloro-6-methylphenyl)-2-[[6-[4-(2-hydroxyethyl)-1-piperazinyl]-2-methyl-4-pyrimidinyl]amino]-5-thiazolecarboxamide, or combinations of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination discloses herein; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, may be warranted. Alternatively, combinations of N-(2-chloro-6-methylphenyl)-2-[[6-[4-(2-hydroxyethyl)-1-piperazinyl]-2-methyl-4-pyrimidinyl]amino]-5-thiazolecarboxamide with a T315I inhibitor may also be warranted.

Therefore, the present invention provides methods of treating an individual suffering from a protein tyrosine kinase-associated disorder such as a BCR-ABL associated disorder, for example, a BCR-ABL-associated cancer, (where such individual is naïve to treatment with a kinase inhibitor (i.e., has not previously been treated with such) or has been treated with one or more kinase inhibitors (for example, has been treated with imatinib or N-(2-chloro-6-methylphenyl)-2-[[6-[4-(2-hydroxyethyl)-1-piperazinyl]-2-methyl-4-pyrimidinyl]amino]-5-thiazolecarboxamide)), comprising determining whether the individual has a mutant BCR-ABL that renders the individual less sensitive to therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and, based on whether the mutant kinase is present, administering to said individual N-(2-chloro-6-methylphenyl)-2-[[6-[4-(2-hydroxyethyl)-1-piperazinyl]-2-methyl-4-pyrimidinyl]amino]-5-thiazolecarboxamide, preferably as an active agent in a pharmaceutical composition at a dose and/or frequency of dosing selected based on said assay (e.g., based on the sensitivity of such mutant(s) relative to with wild-type BCR-ABL kinase), and/or in combination with another protein tyrosine kinase inhibitor, including, for example, imatinib, AMN107, PD180970, GGP76030, AP23464, SKI 606, and/or AZD0530, a tubulin stabilizing agent (e.g., such as paclitaxol, epothilone, taxone, etc.) a farnesyl transferase inhibitor, and/or with another agent suitable for the treatment of said protein tyrosine kinase-associated disorder disclosed herein, said other kinase inhibitor and/or other agent being administered simultaneously or sequentially with the administration of N-(2-chloro-6-methylphenyl)-2-[[6-[4-(2-hydroxyethyl)-1-piperazinyl]-2-methyl-4-pyrimidinyl]amino]-5-thiazolecarboxamide.

According to the present invention, dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. See, e.g., the latest Remington's (Remington's Pharmaceutical Science, Mack Publishing Company, Easton, Pa.)

Therefore, the present invention provides a method of treating an individual suffering from a protein tyrosine kinase-associated disorder (where such individual is naïve to treatment with a kinase inhibitor (i.e., has not previously been treated with such) or has been treated with one or more kinase inhibitors (for example, has been treated with imatinib)), such as a SRC associated disorder (for example, a SRC-associated cancer), comprising: (a) providing a biological sample from said individual (whether as-is or manipulated (such as lysed), for example, to facilitate assaying); (b) assaying said biological sample for the presence of one or more mutant SRC kinase(s); and, based on the results of said assay, such as where one or more mutant SRC kinase(s) is(are) present in said sample, then (c) administering to said individual N-(2-chloro-6-methylphenyl)-2-[[6-[4-(2-hydroxyethyl)-1-piperazinyl]-2-methyl-4-pyrimidinyl]amino]-5-thiazolecarboxamide, preferably as an active agent in a pharmaceutical composition at a dose and/or frequency of dosing selected based on said assay (e.g., based on the sensitivity of such mutant(s) relative to with wild-type SRC kinase), and/or in combination with another protein tyrosine kinase inhibitor, including without limitation imatinib or AMN107, a tubulin stabilizing agent (e.g., such as paclitaxol, epothilone, taxone, etc.) a farnesyl transferase inhibitor such as (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt, and/or with another agent suitable for the treatment of said protein tyrosine kinase-associated disorder disclosed herein, said other kinase inhibitor and/or other agent being administered simultaneously or sequentially with the administration of N-(2-chloro-6-methylphenyl)-2-[[6-[4-(2-hydroxyethyl)-1-piperazinyl]-2-methyl-4-pyrimidinyl]amino]-5-thiazolecarboxamide, wherein, for example, and without limitation:

(1) identification of at least one mutant SRC kinase which is at least partially sensitive to inhibition with N-(2-chloro-6-methylphenyl)-2-[[6-[4-(2-hydroxyethyl)-1-piperazinyl]-2-methyl-4-pyrimidinyl]amino]-5-thiazolecarboxamide may optionally be used to select treatment with a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib preferentially to either kinase inhibitor alone or to other kinase inhibitor(s) (for example, where it is expected that the combination will be effective against said mutant at therapeutically useful doses better tolerated by patients than doses either kinase inhibitor alone or of such other kinase inhibitor(s));

(2) identification of at least one mutant SRC kinase may optionally be used to select the dose of the combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, including increasing or decreasing the dose(s) of the individual agents thereof and/or the frequency of the dosing regimen (either for individuals naïve to N-(2-chloro-6-methylphenyl)-2-[[6-[4-(2-hydroxyethyl)-1-piperazinyl]-2-methyl-4-pyrimidinyl]amino]-5-thiazolecarboxamide or those undergoing treatment with N-(2-chloro-6-methylphenyl)-2-[[6-[4-(2-hydroxyethyl)-1-piperazinyl]-2-methyl-4-pyrimidinyl]amino]-5-thiazolecarboxamide), for example, where the mutant BCR/ABL kinase is inhibited by N-(2-chloro-6-methylphenyl)-2-[[6-[4-(2-hydroxyethyl)-1-piperazinyl]-2-methyl-4-pyrimidinyl]amino]-5-thiazolecarboxamide and/or imatinib to a lesser or greater degree, respectively, relative to WT SRC kinase; and/or

(3) identification of at least one mutant SRC kinase may optionally be used to select co-administration of another agent suitable for treatment of said protein tyrosine kinase-associated disorder in combination with N-(2-chloro-6-methylphenyl)-2-[[6-[4-(2-hydroxyethyl)-1-piperazinyl]-2-methyl-4-pyrimidinyl]amino]-5-thiazolecarboxamide, including and without limitation, a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor, for example, an inhibitor of BCR-ABL, such as imatinib and/or AMN-107, and/or identification of at least one mutant SRC kinase with a sensitivity to N-(2-chloro-6-methylphenyl)-2-[[6-[4-(2-hydroxyethyl)-1-piperazinyl]-2-methyl-4-pyrimidinyl]amino]-5-thiazolecarboxamide decreased relative to other forms of such SRC kinase (e.g., WT SRC kinase) may optionally be used to select a dosing regimen of N-(2-chloro-6-methylphenyl)-2-[[6-[4-(2-hydroxyethyl)-1-piperazinyl]-2-methyl-4-pyrimidinyl]amino]-5-thiazolecarboxamide with an increased dose or increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide relative to a treatment regimen suitable for such other forms of such SRC kinase (e.g., WT); and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, for example, where such agent is at least partially effective in inhibiting said mutant SRC kinase.

### Identification of Mutant BCR-ABL Interactors and Therapeutic Compounds

The present invention provides methods of identifying compounds that interact with, i.e., specifically bind to, a mutant BCR-ABL polypeptide. The compound can be a protein, small molecule, or other agent that can interact with the mutant BCR-ABL polypeptide. Methods of determining whether a test compound can interact with a specific polypeptide are known in the art.

For example, in certain embodiments, the present invention comprises a method of screening for a molecule that interacts with an mutant BCR-ABL amino acid sequence comprising the steps of contacting a population of molecules with the BCR-ABL amino acid sequence, allowing the population of molecules and the BCR-ABL amino acid sequence to interact under conditions that facilitate an interaction, determining the presence of a molecule that interacts with the BCR-ABL amino acid sequence, and then separating molecules that do not interact with the BCR-ABL amino acid sequence from molecules that do. In a specific embodiment, the method further comprises purifying a molecule that interacts with the BCR-ABL amino acid sequence. The identified molecule can be used to modulate a function performed by BCR-ABL.

Screening chemical libraries for molecules which modulate, e.g., inhibit, antagonize, or agonize or mimic, are known in the art. The chemical libraries, for example, can be peptide libraries, peptidomimetic libraries, chemically synthesized libraries, recombinant, e.g., phage display libraries, and in vitro translation-based libraries, other non-peptide synthetic organic libraries (e.g. libraries of 2-phenylaminopyrimidines, quinazolines or pyrazolo-pyrrolo-pyridopyrimidi-nes and the like).

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position can have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

In certain embodiments, one can screen peptide libraries to identify molecules that interact with the mutant BCR-ABL protein sequences. In such methods, peptides that bind to a molecule such as mutant BCR-ABL can be identified by screening libraries that encode a random or controlled collection of amino acids. Peptides encoded by the libraries can be expressed as fusion proteins of bacteriophage coat proteins, the bacteriophage particles can then be screened against the protein of interest.

Accordingly, peptides having a wide variety of uses, such as therapeutic, prognostic or diagnostic reagents, are thus identified without any prior information on the structure of the expected ligand or receptor molecule. Typical peptide libraries and screening methods that can be used to identify molecules that interact with mutant BCR-ABL protein sequences are disclosed for example in U.S. Pat. No. 5,723,286 issued Mar. 3, 1998 and U.S. Pat. No. 5,733,731 issued Mar. 31, 1998, all of which are incorporated herein by reference in their entirety.

Small molecules and ligands that interact with mutant BCR-ABL can be identified through related embodiments of such screening assays. For example, small molecules can be identified that interfere with protein function, including molecules that interfere with a mutant BCR-ABL's ability to mediate phosphorylation and de-phosphorylation.

In certain embodiments, a method of identifying a compound which specifically binds a mutant BCR-ABL as provided herein comprises the steps of: contacting said mutant BCR-ABL with a test compound under conditions favorable to binding; and then determining whether said test compound binds to said mutant BCR-ABL so that a compound which binds to said mutant BCR-ABL can be identified. As the interaction between various Abelson tyrosine kinases and a variety of test compounds have been previously described, skilled artisans are familiar with the conditions conducive to binding.

In certain embodiments, cells will be transfected with a construct encoding the mutant BCR-ABL, contacted with a test compound that is tagged or labelled with a detectable marker and analyzed for the presence bound test compound. In contexts where the transfected cells are observed to preferentially bind the test compound as compared to cells that have not been transfected with a mutant BCR-ABL ARS construct, this indicates that the test compounds is binding to the mutant BCR-ABL protein expressed by those cells. The binding of the compound is typically determined by any one of a wide variety of assays known in the art such as ELISA, RIA, and/or BIAcore assays.

A test compound which binds to a mutant BCR-ABL can be further screened for the inhibition of a biological activity (e.g. tyrosine kinase activity) of said mutant BCR-ABL. Such an embodiment includes, for example determining whether said test compound inhibits the tyrosine kinase activity of the mutant BCR-ABL by utilizing molecular biological protocols to create recombinant contracts whose enzymological and biological properties can be examined directly. A specific biological activity such as resistance to a protein kinase inhibitor can be measured using standard kinase assays and transformation assays. Enzymology is performed for example, by measuring tyrosine kinase activity in vitro or in mutant BCR-ABL expressing cells using standard assays. Such methods typically comprise the steps of examining the kinase activity or growth potential of a BCR-ABL mutant expressing cell line in the absence of a test compound and comparing this to the kinase activity or growth potential of a BCR-ABL mutant expressing cell line in the presence of a test compound, wherein an decrease in the kinase activity or growth potential of the BCR-ABL mutant expressing cell line in the presence of a test compound indicates that said compound can be an inhibitor of the biological activity of said BCR-ABL mutant. For example, BCR-ABL kinase activity can be measured by monitoring the phosphotyrosine content of Crkl using methods known in the art and described in the examples section herein.

Alternative methods for measuring the enzymological and biological property of BCR-ABL mutants variety of assays for measuring the enzymological properties of protein kinases such as Abl are known in the art, and include, for example, those described in Konopka et al., Mol Cell Biol. November 1985; 5(11):3116-23; Davis et al., Mol Cell Biol. January 1985; 5(1):204-13; and Konopka et al., Cell. Jul. 1, 1984; 37(3):1035-42 the contents of which are incorporated herein by reference in their entirety and for all purposes. Using such assays the skilled artisan can measure the enzymological properties of mutant BCR-ABL protein kinases.

A variety of bioassays for measuring the transforming activities of protein kinases such as Abl are known in the art, for example those described in Lugo et al., Science. Mar. 2, 1990; 247(4946):1079-82; Lugo et al., Mol Cell Biol. March 1989; 9(3):1263-70; Klucher et al., Blood. May 15, 1998; 91(10):3927-34; Renshaw et al., Mol Cell Biol. March 1995; 15(3):1286-93; Sitard et al., Blood. Mar. 15, 1994; 83(6):1575-85; Laneuville et al., Cancer Res. Mar. 1, 1994; 54(5):1360-6; Laneuville et al., Blood. Oct. 1, 1992; 80(7):1788-97; Mandanas et al., Leukemia. August 1992; 6(8):796-800; and Laneuville et al., Oncogene. February 1991; 6(2):275-82 the contents of which are incorporated herein by reference in their entirety for all purposes. Using such assays the skilled artisan can measure the phenotype of mutant BCR-ABL protein kinases.

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise a probe that is or can be detectably labeled. Such probe can be an antibody or polynucleotide specific for a mutant BCR-ABL kinase protein or a mutant BCR-ABL kinase gene or message, respectively. Where the kit utilizes nucleic acid hybridization to detect the target nucleic acid, the kit can also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

Kits useful in practicing therapeutic methods disclosed herein can also contain a compound that is capable of inhibiting a mutant BCR-ABL kinase. Specifically contemplated by the invention is a kit comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and another protein tyrosine kinase inhibitor, such as, imatinib, AMN107, PD180970, GGP76030, AP23464, SKI 606, NS-187, and/or AZD0530; an increased dose and/or dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, relative a treatment regimen suitable for such other forms of such BCR-ABL kinase (e.g., wild-type); and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof disclosed herein, useful in treating mammals suffering from a BCR-ABL associated disorder, including mutant BCR-ABL associated disorder. For example, kits useful in identifying a mutant BCR-ABL kinase in a mammalian patient (e.g., a human) suffering from a cancer that is completely or partially resistant to, or has developed complete or partial resistance to, N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, imatinib, or another protein tyrosine kinase inhibitor and where said kits also comprise a therapeutically effective amount of the combination or increased dose or dosing regimen, are contemplated herein.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

The following Exemplary Embodiments of specific aspects for carrying out the present invention are offered for illustrative purposes only, and are not intended to limit the scope of the present invention in any way.

### EXEMPLARY EMBODIMENTS

### Example 1

### Exemplary Methods for Detecting Bcr-Abl Kinase Mutations

N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are two BCR-ABL kinase inhibitors that are effective in treating CML and solid tumors. Provided herein are exemplary combination therapies and dosing regimens that will be useful in treating cancers which are resistant or at least partially resistant to protein tyrosine kinase inhibitor agents, such as imatinib and potentially other kinase inhibitors such as dasatinib, and specifically including cancers involving one or more mutations in BCR-ABL kinase.

A significant aspect of this combination therapy is the detection of the mutations in BCR-ABL kinase. If a mutant BCR-ABL kinase of the present invention is present in a patient, it indicates an individual can be selected for combination therapy, or more aggressive dosing regimens (e.g., higher and/or more frequent doses), or a combination of aggressive dosing regimen and combination therapy. Furthermore, if a specific BCR-ABL kinase mutant is detected, the amount of either or both inhibitors can be increased or decreased in order to enhance the therapeutic effect of the regimen.

There are several methods that can be used to detect a mutant BCR-ABL kinase in cancer patients, particularly CML patients. They include methods for detecting BCR-ABL kinase polynucleotides and BCR-ABL kinase proteins, as well as methods for identifying cells that express BCR-ABL kinase. Detection of certain mutant BCR-ABL kinase in a patient would be diagnostic that such patients either are or will become at least partially resistant to imatinib therapy or N-(2-chloro-6-methylphenyl)-2-[[6-[4-(2-hydroxyethyl)-1-piperazinyl]-2-methyl-4-pyrimidinyl]amino]-5-thiazolecarboxamide therapy. As discussed in detail below, the status of BCR-ABL kinase gene products in patient samples can be analyzed by a variety of protocols well known in the art including, for example, immunohistochemical analysis, the variety of Northern blotting techniques including in situ hybridization, RT-PCR analysis (for example on laser capture micro-dissected samples), western blot analysis, tissue array analysis, microarray analysis, genotyping methods, and mass-spectroscopic methods.

Methods of identifying the nucleic acid and the amino acid of a mutant BCR-ABL kinase are known in the art.

One experimental strategy is to use PCR to amplify a region of the BCR-ABL transcript using primers specific to BCR and ABL, and sequence the PCR fragment directly, or subclone this product and sequence several independent clones in both directions. This strategy allows one to quantify fluctuations in different clones from the same patient over time. Typical methodologies are for such protocols are provided below.

Blood samples can be obtained from patients. RNA is then extracted using TriAgent or TriAzol. cDNA synthesis is performed using MMTV reverse transcriptase. Polymerase chain reaction (PCR) is performed to amplify the cDNA, using primers CM10 (5′-GAAGCTTCTCCCTGACATCCGT-3′) (SEQ ID NO: 3) and 3′ Abl Long KD (5′-CCCCACGGACGCCTTGTTTCCCCAG-3′) (SEQ ID NO: 4). The sequence of BCR-ABL can then be obtained directly by sequencing the resulting product. Alternatively, secondary PCR amplification may be performed to focus on a particular region of the BCR-ABL sequence. For the E507G mutant, for example, a second round of PCR is performed to isolate the kinase domain in addition to enough of BCR/ABL to identify the sequence encoding amino acid position 507 using primers 5′ Abl KD, (5′-GCGCAACAAGCCCACTGTCTATGG-3′) (SEQ ID NO: 5) and 3′ Abl Long KD (SEQ ID NO: 4). The resultant 0.9 Kb fragment is then ligated into pBluescript II KS+ digested with Eco RV. Bacterial transformants are plated on media containing ampicillin and X-gal. Ten white colonies per cDNA, for example, are inoculated into media and miniprep DNA is isolated. One skilled in the art would appreciate that other second round of PCR reactions may be designed to identify other amino acid locations of BCR-ABL that may be of interest by using appropriate primers for the region of interest.

Sequencing of each clone is then performed using M13 universal forward (CGCCAGGGTTTTCCCAGTCACGAC; SEQ ID NO:6) and M13 reverse (AGCGGATAACAATTTCACACAGGA; SEQ ID NO:7) primers. Because two rounds of amplification will be employed, a mutation can be considered present if it was detected on both strands of at least two independent clones per patient.

Alternatively, antibodies that immunospecifically bind to mutant BCR-ABL kinase can be used to detect the presence of a mutant BCR-ABL kinase in a sample. First, mutant BCR-ABL kinase can be generated by site directed mutagenesis. Cell lines expressing these mutant BCR-ABL kinase isoforms will then be created. Next, antibodies against mutant BCR-ABL kinase isoforms will be produced. Expression of BCR-ABL kinase and its mutant isoforms will be documented by Western blot analysis.

Specifically, site directed mutagenesis can be used to create the BCR-ABL kinase mutations (QuickChange Kit, Stratagene, La Jolla, Calif.) and all mutations will be confirmed by bidirectional sequencing (O'Farrell, A. M., et al., Blood, 101:3597-3605 (2003)). Retroviral transduction is performed and Ba/F3 cell lines stably expressing mutant BCR-ABL kinase isoforms are generated by double selection for G418 resistance and IL-3 independent growth (Yee, K. W., et al., Blood, 100:2941-2949 (2002); Yee, K. W., et al., Blood, 104:4202-4209 (2004); Tse, K. F., et al., Leukemia, 14:1766-1776 (2000); Schittenhelm, M. M., et al., manuscript submitted (2005)). Transient transfections of CHO-K1 chinese hamster cell lines with BCR-ABL kinase wild type (“WT”) or mutant isoforms are performed using a lipofection-assay (LipofectAMINE-kit purchased from Gibco-Invitrogen, Carlsbad, Calif.). Cells are treated with N-(2-chloro-6-methylphenyl)-2-[[6-[4-(2-hydroxyethyl)-1-piperazinyl]-2-methyl-4-pyrimidinyl]amino]-5-thiazolecarboxamide 24 hours after transfection (Heinrich, M. C., et al., Journal of Clinical Oncology, 21:4342-4349 (2003)). Alternatively, cells can be treated with any of the combinations outlined herein, or using increased levels of N-(2-chloro-6-methylphenyl)-2-[[6-[4-(2-hydroxyethyl)-1-piperazinyl]-2-methyl-4-pyrimidinyl]amino]-5-thiazolecarboxamide.

An anti-BCR-ABL kinase rabbit polyclonal antibody, an anti-STAT3 mouse monoclonal antibody (both Santa Cruz Biotechnology, Santa Cruz, Calif.), an anti-AKT (polyclonal) rabbit antibody (Cell Signaling Technology, Beverly Mass.) and an anti-MAP kinase 1/2 (Erk 1/2) rabbit monoclonal antibody (Upstate Biotechnology, Lake Placid, N.Y.) can be used at a 1:5000 to 1:2000 dilution. Anti-phosphotyrosine BCR-ABL antibodies (Tyr568/570 and Tyr703), an anti-phosphothreonine/tyrosine MAP kinase (Thr202/Tyr204) antibody, an anti-phosphothreonine (Thr308) and an anti-phosphoserine (Ser473) AKT antibody, an anti-phosphotyrosine (Tyr705) STAT3 antibody and an unspecific anti-phosphotyrosine antibody (clone pY20) are used at dilutions of 1:100 to 1:2000 (all Cell Signaling Technology, Beverly Mass.). Peroxidase conjugated goat anti-mouse antibody and goat anti-rabbit antibody will be used at 1:5000 and 1:10,000 dilutions respectively (BioRad; Hercules, Calif.). Protein A/G PLUS-Agarose immunoprecipitation reagent can be purchased from Santa Cruz Biotechnology (Santa Cruz, Calif.). imatinib, N-(2-chloro-6-methylphenyl)-2-[[6-[4-(2-hydroxyethyl)-1-piperazinyl]-2-methyl-4-pyrimidinyl]amino]-5-thiazolecarboxamide, paclia tubulin stabilizing agent (e.g., pacitaxol, epothilone, taxane, etc.), and another agents useful in combination with N-(2-chloro-6-methylphenyl)-2-[[6-[4-(2-hydroxyethyl)-1-piperazinyl]-2-methyl-4-pyrimidinyl]amino]-5-thiazolecarboxamide, are dissolved in DMSO to create 10 mM stock solutions and be stored at −20° C.

Western blot assays can be conducted as follows. ˜5×10⁷ cells are exposed to varying concentrations of N-(2-chloro-6-methylphenyl)-2-[[6-[4-(2-hydroxyethyl)-1-piperazinyl]-2-methyl-4-pyrimidinyl]amino]-5-thiazolecarboxamide and cultured for 90 minutes at 37° C. in a 5% CO₂ atmosphere. Cell pellets are lysed with 100-150 μL of protein lysis buffer (50 mM Tris, 150 mM NaCl, 1% NP-40, 0.25% deoxycholate with added inhibitors aprotinin, AEBSF, leupeptin, pepstatin, sodium orthovanadate, and sodium pyruvate). 500-2000 microgram of protein from cell lysates are used for immunoprecipitation experiments and 75-200 microgram of protein from cell lysates are used for whole cell protein analysis by western immunoblot assays as previously described in Hoatlin, M. E., et al., Blood, 91:1418-1425 (1998).

In certain contexts, it may be desirable to amplify a specific region in BCR-ABL kinase such as one of the functional domains discussed herein. For example, the region corresponding to the ATP binding pocket and the activation loop domain of BCR-ABL is critical to the selectivity of imatinib and is the region known to harbor the most imatinib-resistant and protein tyrosine kinase inhibitor mutations. Sequencing of this region can most efficiently reveal the patients' clinical profile, and hence the appropriate combination therapy and/or dosing regimen for the patients disorder.

Briefly, RNA is extracted from purified peripheral blood or bone marrow cells with TriReagent-LS (Molecular Research Center, Inc., Cincinnati, Ohio). Total RNA is subjected to RT-PCR by using the same protocol and primers as described supra. PCR products are cloned into the pCR2.1 TA cloning vector (Invitrogen, Carlsbad, Calif.). Both strands of the ˜900 bp region are sequenced with the 5′ ABL primer and M13 forward primer or M13 forward and reverse primer set for the ˜900 bp fragment, on an ABI prism 377 automated DNA sequencer (PE Applied Biosystems, Foster City, Calif.). Sequence analysis is then performed using the ClustalW alignment algorithm). Any detected mutation is then confirmed by analysis of genomic DNA. Briefly, genomic DNA is extracted from purified bone marrow or peripheral blood cells with the QiaAMP Blood Mini Kit (Qiagen, Inc., Valencia, Calif.) using primers specific to intron sequences that flank both sides of the location of the mutation of interest. For example, amplification can be performed by using two primers (5′-GCAGAGTCAGAATCCTTCAG-3′ (SEQ ID NO: 11) and 5′-TTTGTAAAAGGCTGCCCGGC-3′) (SEQ ID NO: 12) which are specific for intron sequences 5′ and 3′ of ABL exon 3, respectively. Other primer pairs for different introns may be designed and are within the skill of the artisan. PCR products are cloned and sequenced.

Additional methods of detecting mutant BCR-ABL kinases is disclosed in O'Hare et al. (Cancer Research, 65(11):4500-5 (2005), which is hereby incorporated by reference in its entirety).

### Example 2

### Exemplary Method of Assessing the Potential Oo the Combination Therapy

The combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib can be studied in mouse models of imatinib-resistant or protein tyrosine kinase inhibitor resistant, BCR-ABL-dependent disease. A series of such pharmacodynamic experiments will help to determine the optimal dosing regimen for different mutant BCR-ABL isoforms in vivo. Pharmacodynamic experiments are well known in the art and one skilled in the art would readily appreciate that such experiments can be modified to alter existing conditions, as applicable. Briefly, severe combined immuno-deficient mice are injected intravenously with Ba/F3 cells expressing different BCR-ABL wild-type or mutant isoforms as well as the firefly luciferase gene. Untreated mice harboring Ba/F3 cells expressing nonmutant or imatinib-resistant BCR-ABL mutants are expected to develop aggressive disease, with massive liver and splenic infiltration, typically resulting in death. To assess the ability of combination therapy, or a modified dosing regimen, to inhibit BCR-ABL in vivo, BCR-ABL kinase activity in splenocyte lysates prepared at various time points after administration of a different single dose of 0, 0.5, 1, 5, and 10 micromoles per liter of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib combination by oral gavage will be assessed. Phosphorylation of the adapter protein CRKL, a known BCR-ABL substrate (T. Oda et al., J. Biol. Chem. 269, 22925 (1994)), will be monitored to gauge the efficacy of the combination therapy. On the basis of a series of such pharmacodynamic experiments, an proper dose of the combination will be chosen for efficacy studies. Then, mice will be documented by bioluminescence imaging before and after dosing. On the basis of a series of such pharmacodynamic experiments, the optimal dosing regimen and/or combination therapy can be identified. Mice are dosed with combination or vehicle alone by gavage for 2 weeks, beginning 3 days after injection of Ba/F3 cells, and disease burden is then assessed by bioluminescence imaging. All vehicle-treated mice are expected to develop progressive disease. In contrast, combination-treated mice harboring nonmutant BCR-ABL or the clinically common imatinib-resistant and protein tyrosine kinase inhibitor resistant mutations described herein are expected to develop less or no progressive disease. It is also expected that different optimal dosing regimens will be identified for different BCR-ABL isoforms. Such dosing difference can be taken into consideration in the treatment of patients with a known BCR-ABL mutation(s).

### Example 3

### Exemplary Method of Assessing the Safety and Efficacy of Protein Tyrosine Kinase Combination Therapy and/or Modified Dosing Regimens

Previous findings have shown that both N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are highly selective for leukemic versus normal hematopoietic cells (B. J. Druker et al., Nature Med. 2. 561 (1996) and N. P. Shah et al., Science 305. 399 (2004)). Such high selectivity demonstrates the high safety and efficacy of these inhibitors, and the expected efficacy of their combination. To assess the efficacy of the combination on human bone marrow progenitors, the compounds are tested in vitro in colony-forming-unit (CFU) assays. A series of concentrations of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib combination, or other combinations disclosed herein, are applied to bone marrow progenitors isolated from healthy volunteers and from CML patients with either imatinib-sensitive (nonmutant BCR-ABL) or imatinib-resistant disease. Furthermore, the blast-forming unit-erythroid (BFU-E) and CFU-granulocyte-monocyte (GM) colonies from CML patient marrow samples will be analyzed by polymerase chain reaction (PCR) analysis in order to detect the sensitivity of selection for growth of rare normal progenitors present in these leukemic marrow samples. Briefly, bone marrow is harvested from clinical subjects. Viable frozen Ficoll-Hypaque-purified mononuclear cells are thawed and grown overnight in Iscove's Media supplemented with 10% Fetal calf serum, 1-glutamine, pen-strep, and stem cell factor (100 ug/ml) at a density of 5×10⁵/ml. After 24 hours, viable cells are quantitated and plated in Methocult media (Cell Signal Technologies, Beverly, Mass.) at 1×10⁴ and 1×10⁵ cells per plate in the presence of 5 nM N-(2-chloro-6-methylphenyl)-2-[[6-[4-(2-hydroxyethyl)-1-piperazinyl]-2-methyl-4-pyrimidinyl]amino]-5-thiazolecarboxamide or vehicle. Experiments are performed in triplicate. On day 11, erythroid blast-forming unit (BFU-E) and granulocyte-macrophage colony forming units (CFU-GM) will be quantitated. On day 14, colonies will be isolated with a pipet tip, and RNA will be isolated using a Qiagen Rneasy kit. A primer complementary to the region of ABL approximately 200 nucleotides downstream of the BCR-ABL mRNA (5′-CGGCATTGCGGGACACAGGCCCATGGTACC; SEQ ID NO:8) junction is annealed to purified RNA. cDNA is synthesized using mouse Moloney leukemia virus (MMLV) reverse transcriptase, and subjecting to 40 cycles of PRC using either a BCR (5′-TGACCAACTCGTGTGTGAAACT; SEQ ID NO:9) or ABL type Ia 5′ primer (GGGGAATTCGCCACCATGTTGGAGATCTGCCTGA; SEQ ID NO:10) as a control for the quality of RNA.

### Example 4

### Exemplary Methods for Measuring of Bcr-Abl Kinase Activity Via the Phosphotyrosine Content of Crkl

The ability of a combination therapy or more aggressive dosing regimen of the present invention to effectively overcome N-(2-chloro-6-methylphenyl)-2-[[6-[4-(2-hydroxyethyl)-1-piperazinyl]-2-methyl-4-pyrimidinyl]amino]-5-thiazolecarboxamide or imatinib resistance, to inhibit BCR-ABL activity, or to inhibit BCR-ABL mutant activity, the phosphotyrosine content of Crkl, an adaptor protein which is specifically and constitutively phosphorylated by BCR-ABL in CML cells can be used (see, e.g. J. ten Hoeve et al., Blood 84, 1731 (1994); T. Oda et al., J. Biol. Chem. 269, 22925 (1994); and G. L. Nichols et al., Blood 84, 2912 (1994)). The phosphotyrosine content of Crkl has been shown to be reproducibly and quantitatively measured in clinical specimens. Crkl binds BCR-ABL directly and plays a functional role in BCR-ABL transformation by linking the kinase signal to downstream effector pathways (see, e.g. K. Senechal et al., J. Bio. Chem. 271, 23255 (1996)). When phosphorylated, Crkl migrates with altered mobility in SDS-PAGE gels and can be quantified using densitometry. Sawyers et al (U.S. Ser. No. 10/171,889, filed Jun. 16, 2002; incorporated herein by reference) have shown that Crkl phosphorylation in primary CML patient cells was inhibited in a dose-dependent manner when exposed to STI-571 and correlated with dephosphorylation of BCR-ABL. Likewise, we have also shown that Crkl phosphorylation was inhibited in a dose-dependent manner when exposed to N-(2-chloro-6-methylphenyl)-2-[[6-[4-(2-hydroxyethyl)-1-piperazinyl]-2-methyl-4-pyrimidinyl]amino]-5-thiazolecarboxamide (data not shown). Thus, such a Crkl assay will allows for an assessment of the enzymatic activity of BCR-ABL protein in a reproducible, quantitative fashion and be a useful means of assessing the ability of a combination therapy or more aggressive dosing regimen of the present invention to effectively overcome imatinib resistance, to inhibit BCR-ABL activity, or to inhibit BCR-ABL mutant activity.

Briefly, cells are lysed in 1% Triton X-100 buffer with protease and phosphatase inhibitors (see, e.g. A. Goga et al., Cell 82, 981 (1995)). Equal amounts of protein, as determined by the BioRad DC protein assay (Bio-RadLaboratories, Hercules, Calif.), are separated by SDS-PAGE, transferred to nitrocellulose and immunoblotted with phosphotyrosine antibody (4G10, Upstate Biotechnologies, Lake Placid, N.Y.), Abl antibody (pex5, (see, e.g. A. Goga et al., Cell 82, 981 (1995)), β-actin antibody (Sigma Chemicals, St. Louis, Mo.) or Crkl antiserum (Santa Cruz Biotechnology, Santa Cruz, Calif.). Immunoreactive bands are visualized by ECL (Amersham Pharmacia Biotech, Piscataway, N.J.). Several exposures are obtained to ensure linear range of signal intensity. Optimal exposures are quantified by densitometry using ImageQuant software (Molecular Dynamics, Sunnyvale, Calif.)).

### Example 5

### Methods for Examining Amplification of the Bcr-Abl Gene in Mammalian Cells

An additional method of assessing the ability of a combination therapy or more aggressive dosing regimen of the present invention to effectively overcome imatinib resistance, to inhibit BCR-ABL activity, or to inhibit BCR-ABL mutant activity is provided. Specifically, dual-color fluorescence in situ hybridization (FISH) experiments can be performed to determine if BCR-ABL gene amplification is effectively diminished. The latter is based upon the appreciation in the art that BCR-ABL amplification is observed in imatinib-resistant and protein tyrosine kinase inhibitor resistant patients. Briefly, interphase and metaphase cells are prepared (see, e.g. E. Abruzzese et al, Cancer Genet. Cytogenet. 105, 164 (1998)) and examined using Locus Specific Identifier (LSI) BCR-ABL dual color translocation probe (Lysis, Inc., Downers Grove, Ill.)). Cytogenetic and FISH characterization of metaphase spreads can be observed to assess if an inverted duplicate Ph-chromosome with interstitial amplification of the BCR-ABL fusion gene is present.

Alternatively, quantitative PCR analysis of genomic DNA obtained from patients can be used to assess if BCR-ABL gene amplification is present. Briefly, genomic DNA can be extracted from purified bone marrow or peripheral blood cells with the QiaAMP Blood Mini Kit (Qiagen, Inc., Valencia, Calif.). 10 ng of total genomic DNA is subjected to real-time PCR analysis with the iCycler iQ system (Bio-Rad Laboratories, Hercules, Calif.). A 361-bp cDNA fragment including ABL exon 3 is amplified using two primers (5′-GCAGAGTCAGAATCCTTCAG-3′ (SEQ ID NO:11) and 5′-TTTGTAAAAGGCTGCCCGGC-3′ (SEQ ID NO:12)) which are specific for intron sequences 5′ and 3′ of ABL exon 3, respectively. A 472-bp cDNA fragment of glyceraldehyde-3-phosphate dehydrogenase (GAPDH) is amplified using two primers (5′-TTCACCACCATGGAGAAGGC-3′ (SEQ ID NO: 13) and 5′-CAGGAAATGAGCTTGACAAA-3′ (SEQ ID NO: 14)) which are specific for sequences in exon 5 and exon 8 of GAPDH, respectively. Fold increase in ABL copy number can be determined by calculating the difference between threshold cycle numbers of ABL and GAPDH for each sample (DCt). A control can be used as a reference sample, DCt from each sample can be subtracted from DCt of control to determine D(DCt). Fold increase is then calculated as 2^{-D(DCt)}.

### Example 6

### Art Accepted Methods for Measuring Enzymological and Biological Properties of Bcr-Abl Mutants

A variety of assays for measuring the enzymological properties of protein kinases such as Abl are known in the art, for example those described in Konopka et al., Mol Cell Biol. November 1985; 5(11):3116-23; Davis et al., Mol Cell Biol., January 1985; 5(1):204-13; and Konopka et al., Cell. Jul. 1, 1984; 37(3):1035-42 the contents of which are incorporated herein by reference. Using such assays the skilled artisan can measure the enzymological properties of mutant BCR-ABL protein kinases and to assess the ability of a combination therapy or more aggressive dosing regimen of the present invention to effectively overcome imatinib resistance, to inhibit BCR-ABL activity, or to inhibit BCR-ABL mutant activity.

A variety of bioassays for measuring the transforming activities of protein kinases such as Abl are known in the art, for example those described in Lugo et al., Science. Mar. 2, 1990; 247(4946):1079-82; Lugo et al., Mol Cell Biol. March 1989; 9(3):1263-70; Klucher et al., Blood. May 15, 1998; 91(10):3927-34; Renshaw et al., Mol Cell Biol. March 1995; 15(3):1286-93; Sitard et al., Blood. Mar. 15, 1994; 83(6):1575-85; Laneuville et al., Cancer Res. Mar. 1, 1994; 54(5):1360-6; Laneuville et al., Blood. Oct. 1, 1992; 80(7):1788-97; Mandanas et al., Leukemia. August 1992; 6(8):796-800; and Laneuville et al., Oncogene. February 1991; 6(2):275-82 the contents of which are incorporated herein by reference. Using such assays the skilled artisan can measure the phenotype of mutant BCR-Abl protein kinases.

Additional methods are disclosed in O'Hare et al. (Cancer Research, 65(11):4500-5 (2005), which is hereby incorporated by reference in its entirety.

### Example 7

### Response to Dasatinib after Imatinib Failure According to Type of Preexisting Bcr-Abl Mutations

Dasatinib is a novel, oral, multi-targeted kinase inhibitor of BCR-ABL and SRC kinases with preclinical activity against 20/21 imatinib resistant BCR-ABL mutations and clinical phase I/II efficacy in patients with chronic myelogenous leukemia (CML) and BCR-ABL positive acute lymphoblastic leukemia (ALL). The relationship between type of preexisting BCR-ABL mutations associated with imatinib resistance and efficacy of dasatinib in patients (pts) with CML and ALL was assessed. 872 peripheral blood samples from 394 patients (53% male, median age 60 yrs, range 17-85) who had been enrolled in international phase II studies investigating the activity of 70 mg dasatinib BID after imatinib failure were investigated (chronic phase, CP, n=198; accelerated phase, AP, n=78; myeloid blast crisis, MyBC, n=53; lymphoid blast crisis, LyBC, or ALL, n=65). Screening for BCR-ABL mutations was performed by D-HPLC combined with DNA sequencing. During follow up, patients were monitored in 3-monthly intervals by RQ-PCR for BCR-ABL mRNA transcripts and by mutation analysis to determine the quantitative course of the preexisting mutation or the emergence of new mutations. Hematologic and cytogenetic response data have been collected sequentially for a median of 8 months (range, 2-11) after start of therapy. Prior to dasatinib, 46 different BCR-ABL mutations involving 36 amino acids were detected in 202/394 patients (51%). 162 patients showed one, 33 patients two, 6 patients three, and 1 pt four mutations. Mutations were observed in 84 patients in CP (42%), 47 patients in AP (60%), 23 patients in MyBC (43%), and 48 patients in LyBC and ALL (74%). In patients with mutations, hematologic response was 91% in CP, 62% in AP, 41% in MyBC, and 34% in LyBC/ALL (p<0.01), major and complete cytogenetic response did not differ significantly (47% and 34% in CP, 35% and 27% in AP, 33% and 28% in MyBC, 53% and 51% in LyBC/ALL). Major cytogenetic response rates were comparable in patients bearing no mutations (44%), mutations within the P-Loop (43%), SH2 domain (47%), activation loop (56%), or other sites (49%), but not for T315I (0%, p<0.001). Sorting individual patients by the underlying mutation and cellular IC50 values of dasatinib revealed clearly higher hematologic and cytogenetic response rates in those with lower IC50 values. In line with the virtual insensitivity to dasatinib in vitro, none of the 17 patients carrying a T315I mutation showed any hematologic response. Two distinct patterns of response were observed: (1) a parallel decrease of the BCR-ABL load and the mutated clone or (2) a decrease of the BCR-ABL load followed by a decrease of the mutated clone after a delay of up to 4-6 months. Up until now 5 patients developed new mutations associated with dasatinib resistance (T315I, n=2, AP and MyBC pts; F317L+F486S, n=2, LyBC and MyBC pts; E507G, n=1, CP pt). We conclude that dasatinib is capable of inducing hematologic and cytogenetic remissions in a significant proportion of imatinib resistant patients harboring BCR-ABL mutations, except T315I. Response dynamics depend on the individual type of mutation which may be a basis for individual dose adaptation according to the mutation pattern.

### Example 8

### Method of Creating N- and C-Terminal Deletion Mutants Corresponding to the Mutant Bcr/Abl Polypeptide of the Present Invention

As described elsewhere herein, the present invention encompasses the creation of N- and C-terminal deletion mutants, in addition to any combination of N- and C-terminal deletions thereof, corresponding to the mutant BCR/ABL polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length mutant BCR/ABL polypeptide sequence, appropriate primers of about 15-25 nucleotides derived from the desired 5′ and 3′ positions of SEQ ID NO:1 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

In preferred embodiments, the following N-terminal BCR/ABL deletion polypeptides are encompassed by the present invention: M1-R1130, L2-R1130, E3-R1130, I4-R1130, C5-R1130, L6-R1130, K7-R1130, L8-R1130, V9-R1130, G10-R1130, C11-R1130, K12-R1130, S13-R1130, K14-R1130, K15-R1130, G16-R1130, L17-R1130, S18-R1130, S19-R1130, S20-R1130, S21-R1130, S22-R1130, C23-R1130, Y24-R1130, L25-R1130, E26-R1130, E27-R1130, A28-R1130, L29-R1130, Q30-R1130, R31-R1130, P32-R1130, V33-R1130, A34-R1130, S35-R1130, D36-R1130, F37-R1130, E38-R1130, P39-R1130, Q40-R1130, G41-R1130, L42-R1130, S43-R1130, E44-R1130, A45-R1130, A46-R1130, R47-R1130, W48-R1130, N49-R1130, S50-R1130, K51-R1130, E52-R1130, N53-R1130, L54-R130, L55-R1130, A56-R1130, G57-R1130, P58-R1130, S59-R1130, E60-R1130, N61-R1130, D62-R1130, P63-R1130, N64-R130, L65-R1130, F66-R1130, V67-R1130, A68-R1130, L69-R1130, Y70-R1130, D71-R1130, F72-R1130, V73-R1130, A74-R1130, S75-R1130, G76-R1130, D77-R1130, N78-R1130, T79-R1130, L80-R1130, S81-R1130, I82-R1130, T83-R1130, K84-R1130, G85-R1130, E86-R1130, K87-R1130, L88-R1130, R89-R1130, V90-R1130, L91-R1130, G92-R1130, Y93-R1130, N94-R1130, H95-R1130, N96-R1130, G97-R1130, E98-R1130, W99-R1130, C100-R1130, E101-R1130, A102-R1130, Q103-R1130, T104-R1130, K105-R1130, N106-R1130, G107-R1130, Q108-R1130, G109-R1130, W110-R1130, V111-R130, P112-R1130, S113-R1130, N114-R1130, Y115-R1130, I116-R1130, T117-R130, P118-R1130, V119-R1130, N120-R1130, S121-R130, L122-R1130, E123-R1130, K124-R1130, H125-R1130, S126-R1130, W127-R1130, Y128-R1130, H129-R1130, G130-R1130, P131-R1130, V132-R1130, S133-R1130, R134-R1130, N135-R1130, A136-R1130, A137-R1130, E138-R1130, Y139-R1130, P140-R1130, L141-R1130, S142-R1130, S143-R1130, G144-R1130, I145-R1130, N146-R1130, G147-R1130, S148-R1130, F149-R1130, L150-R1130, V151-R1130, R152-R1130, E153-R1130, S154-R1130, E155-R1130, S156-R1130, S157-R1130, P158-R1130, S159-R1130, Q160-R1130, R161-R1130, S162-R1130, I163-R1130, S164-R1130, L165-R1130, R166-R1130, Y167-R1130, E168-R1130, G169-R1130, R170-R1130, V171-R1130, Y172-R1130, H173-R1130, Y174-R1130, R175-R1130, I176-R1130, N177-R1130, T178-R1130, A179-R1130, S180-R1130, D181-R1130, G182-R1130, K183-R1130, L184-R1130, Y185-R1130, V186-R1130, S187-R1130, S188-R1130, E189-R1130, S190-R1130, R191-R1130, F192-R1130, N193-R1130, T194-R1130, L195-R1130, A196-R1130, E197-R1130, L198-R1130, V199-R1130, H200-R1130, H201-R1130, H202-R1130, S203-R1130, T204-R1130, V205-R1130, A206-R1130, D207-R1130, G208-R1130, L209-R1130, I210-R1130, T211-R1130, T212-R1130, L213-R1130, H214-R1130, Y215-R1130, P216-R1130, A217-R1130, P218-R1130, K219-R1130, R220-R1130, N221-R1130, K222-R1130, P223-R1130, T224-R1130, V225-R1130, Y226-R1130, G227-R1130, V228-R1130, S229-R1130, P230-R1130, N231-R1130, Y232-R1130, D233-R1130, K234-R1130, W235-R1130, E236-R1130, M237-R1130, E238-R1130, R239-R1130, T240-R1130, D241-R1130, I242-R1130, T243-R1130, M244-R1130, K245-R1130, H246-R1130, K247-R1130, L248-R1130, G249-R1130, G250-R1130, G251-R1130, Q252-R1130, Y253-R1130, G254-R1130, E255-R1130, V256-R1130, Y257-R1130, E258-R1130, G259-R1130, V260-R1130, W261-R1130, K262-R1130, K263-R1130, Y264-R1130, S265-R1130, L266-R1130, T267-R1130, V268-R1130, A269-R1130, V270-R1130, K271-R1130, T272-R1130, L273-R1130, K274-R1130, E275-R1130, D276-R1130, T277-R1130, M278-R1130, E279-R1130, V280-R1130, E281-R1130, E282-R1130, F283-R1130, L284-R1130, K285-R1130, E286-R1130, A287-R1130, A288-R1130, V289-R1130, M290-R1130, K291-R1130, E292-R1130, I293-R1130, K294-R1130, H295-R1130, P296-R1130, N297-R1130, L298-R1130, V299-R1130, Q300-R1130, L301-R1130, L302-R1130, G303-R1130, V304-R1130, C305-R1130, T306-R1130, R307-R1130, E308-R1130, P309-R1130, P310-R1130, F311-R1130, Y312-R1130, I313-R1130, I314-R1130, T315-R1130, E316-R1130, F317-R1130, M318-R1130, T319-R1130, Y320-R1130, G321-R1130, N322-R1130, L323-R1130, L324-R1130, D325-R1130, Y326-R1130, L327-R1130, R328-R1130, E329-R1130, C330-R1130, N331-R1130, R332-R1130, Q333-R1130, E334-R1130, V335-R1130, N336-R1130, A337-R1130, V338-R1130, V339-R1130, L340-R1130, L341-R1130, Y342-R1130, M343-R1130, A344-R1130, T345-R1130, Q346-R1130, I347-R1130, S348-R1130, S349-R1130, A350-R1130, M351-R1130, E352-R1130, Y353-R1130, L354-R1130, E355-R1130, K356-R1130, K357-R1130, N358-R1130, F359-R1130, I360-R1130, H361-R1130, R362-R1130, D363-R1130, L364-R1130, A365-R1130, A366-R1130, R367-R1130, N368-R1130, C369-R1130, L370-R1130, V371-R1130, G372-R1130, E373-R1130, N374-R1130, H375-R1130, L376-R1130, V377-R1130, K378-R1130, V379-R1130, A380-R1130, D381-R1130, F382-R1130, G383-R1130, L384-R1130, S385-R1130, R386-R1130, L387-R1130, M388-R1130, T389-R1130, G390-R1130, D391-R1130, T392-R1130, Y393-R1130, T394-R1130, A395-R1130, H396-R1130, A397-R1130, G398-R1130, A399-R1130, K400-R1130, F401-R1130, P402-R1130, I403-R1130, K404-R1130, W405-R1130, T406-R1130, A407-R1130, P408-R1130, E409-R1130, S410-R1130, L411-R1130, A412-R1130, Y413-R1130, N414-R1130, K415-R1130, F416-R1130, S417-R1130, 1418-R1130, K419-R1130, S420-R1130, D421-R1130, V422-R1130, W423-R1130, A424-R1130, F425-R1130, G426-R1130, V427-R1130, L428-R1130, L429-R1130, W430-R1130, E431-R1130, I432-R1130, A433-R1130, T434-R1130, Y435-R1130, G436-R1130, M437-R1130, S438-R1130, P439-R1130, Y440-R1130, P441-R1130, G442-R1130, I443-R1130, D444-R1130, R445-R1130, S446-R1130, Q447-R1130, V448-R1130, Y449-R1130, E450-R1130, L451-R1130, L452-R1130, E453-R1130, K454-R1130, D455-R1130, Y456-R1130, R457-R1130, M458-R1130, K459-R1130, R460-R1130, P461-R1130, E462-R1130, G463-R1130, C464-R1130, P465-R1130, E466-R1130, K467-R1130, V468-R1130, Y469-R1130, E470-R1130, L471-R1130, M472-R1130, R473-R1130, A474-R1130, C475-R1130, W476-R1130, Q477-R1130, W478-R1130, N479-R1130, P480-R1130, S481-R1130, D482-R1130, R483-R1130, P484-R1130, S485-R1130, F486-R1130, A487-R1130, E488-R1130, I489-R1130, H490-R1130, Q491-R1130, A492-R1130, F493-R1130, E494-R1130, T495-R1130, M496-R1130, F497-R1130, Q498-R1130, E499-R1130, S500-R1130, S501-R1130, 1502-R1130, S503-R1130, D504-R1130, E505-R1130, V506-R1130, E507-R1130, K508-R1130, E509-R1130, L510-R1130, G511-R1130, K512-R1130, Q513-R1130, G514-R1130, V515-R130, R516-R1130, G517-R1130, A518-R1130, V519-R1130, T520-R1130, T521-R1130, L522-R1130, L523-R1130, Q524-R1130, A525-R1130, P526-R1130, E527-R1130, L528-R1130, P529-R1130, T530-R1130, K531-R1130, T532-R1130, R533-R1130, T534-R1130, S535-R1130, R536-R1130, R537-R1130, A538-R1130, A539-R1130, E540-R1130, H541-R1130, R542-R1130, D543-R1130, T544-R1130, T545-R1130, D546-R1130, V547-R1130, P548-R1130, E549-R1130, M550-R1130, P551-R1130, H552-R1130, S553-R1130, K554-R1130, G555-R1130, Q556-R1130, G557-R1130, E558-R1130, S559-R1130, D560-R1130, P561-R1130, L562-R1130, D563-R1130, H564-R1130, E565-R1130, P566-R1130, A567-R1130, V568-R1130, S569-R1130, P570-R1130, L571-R1130, L572-R1130, P573-R1130, R574-R1130, K575-R1130, E576-R1130, R577-R1130, G578-R1130, P579-R1130, P580-R1130, E581-R1130, G582-R1130, G583-R1130, L584-R1130, N585-R1130, E586-R1130, D587-R1130, E588-R1130, R589-R1130, L590-R1130, L591-R1130, P592-R1130, K593-R1130, D594-R1130, K595-R1130, K596-R1130, T597-R1130, N598-R1130, L599-R1130, F600-R1130, S601-R1130, A602-R1130, L603-R1130, 1604-R1130, K605-R1130, K606-R1130, K607-R1130, K608-R1130, K609-R1130, T610-R1130, A611-R1130, P612-R1130, T613-R1130, P614-R1130, P615-R1130, K616-R1130, R617-R1130, S618-R1130, S619-R1130, S620-R1130, F621-R1130, R622-R1130, E623-R1130, M624-R1130, D625-R1130, G626-R1130, Q627-R1130, P628-R1130, E629-R1130, R630-R1130, R631-R1130, G632-R1130, A633-R1130, G634-R1130, E635-R1130, E636-R1130, E637-R1130, G638-R1130, R639-R1130, D640-R1130, 1641-R1130, S642-R1130, N643-R1130, G644-R1130, A645-R1130, L646-R1130, A647-R1130, F648-R1130, T649-R1130, P650-R1130, L651-R1130, D652-R1130, T653-R1130, A654-R1130, D655-R1130, P656-R1130, A657-R1130, K658-R1130, S659-R1130, P660-R1130, K661-R1130, P662-R1130, S663-R1130, N664-R1130, G665-R1130, A666-R1130, G667-R1130, V668-R1130, P669-R1130, N670-R1130, G671-R1130, A672-R1130, L673-R1130, R674-R1130, E675-R1130, S676-R1130, G677-R1130, G678-R1130, S679-R1130, G680-R1130, F681-R1130, R682-R1130, S683-R1130, P684-R1130, H685-R1130, L686-R1130, W687-R1130, K688-R1130, K689-R1130, S690-R1130, S691-R1130, T692-R1130, L693-R1130, T694-R1130, S695-R1130, S696-R1130, R697-R1130, L698-R1130, A699-R1130, T700-R1130, G701-R1130, E702-R1130, E703-R1130, E704-R1130, G705-R1130, G706-R1130, G707-R1130, S708-R1130, S709-R1130, S710-R1130, K711-R1130, R712-R1130, F713-R1130, L714-R1130, R715-R1130, S716-R1130, C717-R1130, S718-R1130, V719-R1130, S720-R1130, C721-R1130, V722-R1130, P723-R1130, H724-R1130, G725-R1130, A726-R1130, K727-R1130, D728-R1130, T729-R1130, E730-R1130, W731-R1130, R732-R1130, S733-R1130, V734-R1130, T735-R1130, L736-R1130, P737-R1130, R738-R1130, D739-R1130, L740-R1130, Q741-R1130, S742-R1130, T743-R1130, G744-R1130, R745-R1130, Q746-R1130, F747-R1130, D748-R1130, S749-R1130, S750-R1130, T751-R1130, F752-R1130, G753-R1130, G754-R1130, H755-R1130, K756-R1130, S757-R1130, E758-R1130, K759-R1130, P760-R1130, A761-R1130, L762-R1130, P763-R1130, R764-R1130, K765-R1130, R766-R1130, A767-R1130, G768-R1130, E769-R1130, N770-R1130, R771-R1130, S772-R1130, D773-R1130, Q774-R1130, V775-R1130, T776-R1130, R777-R1130, G778-R1130, T779-R1130, V780-R1130, T781-R1130, P782-R1130, P783-R1130, P784-R1130, R785-R1130, L786-R1130, V787-R1130, K788-R1130, K789-R1130, N790-R1130, E791-R1130, E792-R1130, A793-R1130, A794-R1130, D795-R1130, E796-R1130, V797-R1130, F798-R1130, K799-R1130, D800-R1130, 1801-R1130, M802-R1130, E803-R1130, S804-R1130, S805-R1130, P806-R1130, G807-R1130, S808-R1130, S809-R1130, P810-R1130, P811-R1130, N812-R1130, L813-R1130, T814-R1130, P815-R1130, K816-R1130, P817-R1130, L818-R1130, R819-R1130, R820-R1130, Q821-R1130, V822-R1130, T823-R1130, V824-R1130, A825-R1130, P826-R1130, A827-R1130, S828-R1130, G829-R1130, L830-R1130, P831-R1130, H832-R1130, K833-R1130, E834-R1130, E835-R1130, A836-R1130, W837-R1130, K838-R1130, G839-R1130, S840-R1130, A841-R1130, L842-R1130, G843-R1130, T844-R1130, P845-R1130, A846-R1130, A847-R1130, A848-R1130, E849-R1130, P850-R1130, V851-R1130, T852-R1130, P853-R1130, T854-R1130, S855-R1130, K856-R1130, A857-R1130, G858-R1130, S859-R1130, G860-R1130, A861-R1130, P862-R1130, R863-R1130, G864-R1130, T865-R1130, S866-R1130, K867-R1130, G868-R1130, P869-R1130, A870-R1130, E871-R1130, E872-R1130, S873-R1130, R874-R1130, V875-R1130, R876-R1130, R877-R1130, H878-R1130, K879-R1130, H880-R1130, S881-R1130, S882-R1130, E883-R1130, S884-R1130, P885-R1130, G886-R1130, R887-R1130, D888-R1130, K889-R1130, G890-R1130, K891-R1130, L892-R1130, S893-R1130, K894-R1130, L895-R1130, K896-R1130, P897-R1130, A898-R1130, P899-R1130, P900-R1130, P901-R1130, P902-R1130, P903-R1130, A904-R1130, A905-R1130, S906-R1130, A907-R1130, G908-R1130, K909-R1130, A910-R1130, G911-R1130, G912-R1130, K913-R1130, P914-R1130, S915-R1130, Q916-R1130, R917-R1130, P918-R1130, G919-R1130, Q920-R1130, E921-R1130, A922-R1130, A923-R1130, G924-R1130, E925-R1130, A926-R1130, V927-R1130, L928-R1130, G929-R1130, A930-R1130, K931-R1130, T932-R1130, K933-R1130, A934-R1130, T935-R1130, S936-R1130, L937-R1130, V938-R1130, D939-R1130, A940-R1130, V941-R1130, N942-R1130, S943-R1130, D944-R1130, A945-R1130, A946-R1130, K947-R1130, P948-R1130, S949-R1130, Q950-R1130, P951-R1130, A952-R1130, E953-R1130, G954-R1130, L955-R1130, K956-R1130, K957-R1130, P958-R1130, V959-R1130, L960-R1130, P961-R1130, A962-R1130, T963-R1130, P964-R1130, K965-R1130, P966-R1130, H967-R1130, P968-R1130, A969-R1130, K970-R1130, P971-R1130, S972-R1130, G973-R1130, T974-R1130, P975-R1130, 1976-R1130, S977-R1130, P978-R1130, A979-R1130, P980-R1130, V981-R1130, P982-R1130, L983-R1130, S984-R1130, T985-R1130, L986-R1130, P987-R1130, S988-R1130, A989-R1130, S990-R1130, S991-R1130, A992-R1130, L993-R1130, A994-R1130, G995-R1130, D996-R1130, Q997-R1130, P998-R1130, S999-R1130, S1000-R1130, T1001-R1130, A1002-R1130, F1003-R1130, I1004-R1130, P1005-R1130, L1006-R1130, I1007-R1130, S1008-R1130, T1009-R1130, R1010-R1130, V1011-R1130, S1012-R1130, L1013-R1130, R1014-R1130, K1015-R1130, T1016-R1130, R1017-R1130, Q1018-R1130, P1019-R1130, P1020-R1130, E1021-R1130, R1022-R1130, A1023-R1130, S1024-R1130, G1025-R1130, A1026-R1130, I1027-R1130, T1028-R1130, K1029-R1130, G1030-R1130, V1031-R1130, V1032-R1130, L1033-R1130, D1034-R1130, S1035-R1130, T1036-R1130, E1037-R1130, A1038-R1130, L1039-R1130, C1040-R1130, L1041-R1130, A1042-R1130, I1043-R1130, S1044-R1130, G1045-R1130, N1046-R1130, S1047-R1130, E1048-R1130, Q1049-R1130, M1050-R1130, A1051-R1130, S1052-R1130, H1053-R1130, S1054-R1130, A1055-R1130, V1056-R1130, L1057-R1130, E1058-R1130, A1059-R1130, G1060-R1130, K1061-R1130, N1062-R1130, L1063-R1130, Y1064-R1130, T1065-R1130, F1066-R1130, C1067-R1130, V1068-R1130, S1069-R1130, Y1070-R1130, V1071-R1130, D1072-R1130, S1073-R1130, I1074-R1130, Q1075-R1130, Q1076-R1130, M1077-R1130, R1078-R1130, N1079-R1130, K1080-R1130, F1081-R1130, A1082-R1130, F1083-R1130, R1084-R1130, E1085-R1130, A1086-R1130, I1087-R1130, N1088-R1130, K1089-R1130, L1090-R1130, E1091-R1130, N1092-R1130, N1093-R1130, L1094-R1130, R1095-R1130, E1096-R1130, L1097-R1130, Q1098-R1130, I1099-R1130, C1100-R1130, P1101-R1130, A1102-R1130, S1103-R1130, A1104-R1130, G1105-R1130, S1106-R1130, G1107-R1130, P1108-R1130, A1109-R1130, A1110-R1130, T1111-R1130, Q1112-R1130, D1113-R1130, F1114-R1130, S1115-R1130, K1116-R1130, L1117-R1130, L1118-R1130, S1119-R1130, S1120-R1130, V1121-R1130, K1122-R1130, E1123-R1130, and/or I1124-R1130 of SEQ ID NO:2. Polynucleotide sequences encoding these polypeptides are also provided. The present invention also encompasses the use of these N-terminal BCR/ABL deletion polypeptides as immunogenic and/or antigenic epitopes as described elsewhere herein.

In preferred embodiments, the following C-terminal BCR/ABL deletion polypeptides are encompassed by the present invention: M1-R1130, M1-Q1129, M1-V1128, M1-I1127, M1-D1126, M1-S1125, M1-I1124, M1-E1123, M1-K1122, M1-V1121, M1-S1120, M1-S1119, M1-L1118, M1-L1117, M1-K1116, M1-S1115, M1-F1114, M1-D1113, M1-Q1112, M1-T1111, M1-A1110, M1-A1109, M1-P1108, M1-G1107, M1-S1106, M1-G1105, M1-A1104, M1-S1103, M1-A1102, M1-P1101, M1-C1000, M1-I1099, M1-Q1098, M1-L1097, M1-E1096, M1-R1095, M1-L1094, M1-N1093, M1-N1092, M1-E1091, M1-L1090, M1-K1089, M1-N1088, M1-I1087, M1-A1086, M1-E1085, M1-R1084, M1-F1083, M1-A1082, M1-F1081, M1-K1080, M1-N1079, M1-R1078, M1-M1077, M1-Q1076, M1-Q1075, M1-I1074, M1-S1073, M1-D1072, M1-V1071, M1-Y1070, M1-S1069, M1-V1068, M1-C1067, M1-F1066, M1-T1065, M1-Y1064, M1-L1063, M1-N1062, M1-K1061, M1-G1060, M1-A1059, M1-E1058, M1-L1057, M1-V1056, M1-A1055, M1-S1054, M1-H1053, M1-S1052, M1-A1051, M1-M1050, M1-Q1049, M1-E1048, M1-S1047, M1-N1046, M1-G1045, M1-S1044, M1-I1043, M1-A1042, M1-L1041, M1-C1040, M1-L1039, M1-A1038, M1-E1037, M1-T1036, M1-S1035, M1-D1034, M1-L1033, M1-V1032, M1-V1031, M1-G1030, M1-K1029, M1-T1028, M1-I1027, M1-A1026, M1-G1025, M1-S1024, M1-A1023, M1-R1022, M1-E1021, M1-P1020, M1-P1019, M1-Q1018, M1-R1017, M1-T1016, M1-K1015, M1-R1014, M1-L1013, M1-S1012, M1-V1011, M1-R100, M1-T1009, M1-S1008, M1-I1007, M1-L1006, M1-P1005, M1-I1004, M1-F1003, M1-A1002, M1-T1001, M1-S1000, M1-S999, M1-P998, M1-Q997, M1-D996, M1-G995, M1-A994, M1-L993, M1-A992, M1-S991, M1-S990, M1-A989, M1-S988, M1-P987, M1-L986, M1-T985, M1-S984, M1-L983, M1-P982, M1-V981, M1-P980, M1-A979, M1-P978, M1-S977, M1-I976, M1-P975, M1-T974, M1-G973, M1-S972, M1-P971, M1-K970, M1-A969, M1-P968, M1-H967, M1-P966, M1-K965, M1-P964, M1-T963, M1-A962, M1-P961, M1-L960, M1-V959, M1-P958, M1-K957, M1-K956, M1-L955, M1-G954, M1-E953, M1-A952, M1-P951, M1-Q950, M1-S949, M1-P948, M1-K947, M1-A946, M1-A945, M1-D944, M1-S943, M1-N942, M1-V941, M1-A940, M1-D939, M1-V938, M1-L937, M1-S936, M1-T935, M1-A934, M1-K933, M1-T932, M1-K931, M1-A930, M1-G929, M1-L928, M1-V927, M1-A926, M1-E925, M1-G924, M1-A923, M1-A922, M1-E921, M1-Q920, M1-G919, M1-P918, M1-R917, M1-Q916, M1-S915, M1-P914, M1-K913, M1-G912, M1-G911, M1-A910, M1-K909, M1-G908, M1-A907, M1-S906, M1-A905, M1-A904, M1-P903, M1-P902, M1-P901, M1-P900, M1-P899, M1-A898, M1-P897, M1-K896, M1-L895, M1-K894, M1-S893, M1-L892, M1-K891, M1-G890, M1-K889, M1-D888, M1-R887, M1-G886, M1-P885, M1-S884, M1-E883, M1-S882, M1-S881, M1-H880, M1-K879, M1-H878, M1-R877, M1-R876, M1-V875, M1-R874, M1-S873, M1-E872, M1-E871, M1-A870, M1-P869, M1-G868, M1-K867, M1-S866, M1-T865, M1-G864, M1-R863, M1-P862, M1-A861, M1-G860, M1-S859, M1-G858, M1-A857, M1-K856, M1-S855, M1-T854, M1-P853, M1-T852, M1-V851, M1-P850, M1-E849, M1-A848, M1-A847, M1-A846, M1-P845, M1-T844, M1-G843, M1-L842, M1-A841, M1-S840, M1-G839, M1-K838, M1-W837, M1-A836, M1-E835, M1-E834, M1-K833, M1-H832, M1-P831, M1-L830, M1-G829, M1-S828, M1-A827, M1-P826, M1-A825, M1-V824, M1-T823, M1-V822, M1-Q821, M1-R820, M1-R819, M1-L818, M1-P817, M1-K816, M1-P815, M1-T814, M1-L813, M1-N812, M1-P811, M1-P810, M1-S809, M1-S808, M1-G807, M1-P806, M1-S805, M1-S804, M1-E803, M1-M802, M1-I801, M1-D800, M1-K799, M1-F798, M1-V797, M1-E796, M1-D795, M1-A794, M1-A793, M1-E792, M1-E791, M1-N790, M1-K789, M1-K788, M1-V787, M1-L786, M1-R785, M1-P784, M1-P783, M1-P782, M1-T781, M1-V780, M1-T779, M1-G778, M1-R777, M1-T776, M1-V775, M1-Q774, M1-D773, M1-S772, M1-R771, M1-N770, M1-E769, M1-G768, M1-A767, M1-R766, M1-K765, M1-R764, M1-P763, M1-L762, M1-A761, M1-P760, M1-K759, M1-E758, M1-S757, M1-K756, M1-H755, M1-G754, M1-G753, M1-F752, M1-T751, M1-S750, M1-S749, M1-D748, M1-F747, M1-Q746, M1-R745, M1-G744, M1-T743, M1-S742, M1-Q741, M1-L740, M1-D739, M1-R738, M1-P737, M1-L736, M1-T735, M1-V734, M1-S733, M1-R732, M1-W731, M1-E730, M1-T729, M1-D728, M1-K727, M1-A726, M1-G725, M1-H724, M1-P723, M1-V722, M1-C721, M1-S720, M1-V719, M1-S718, M1-C717, M1-S716, M1-R715, M1-L714, M1-F713, M1-R712, M1-K711, M1-S710, M1-S709, M1-S708, M1-G707, M1-G706, M1-G705, M1-E704, M1-E703, M1-E702, M1-G701, M1-T700, M1-A699, M1-L698, M1-R697, M1-S696, M1-S695, M1-T694, M1-L693, M1-T692, M1-S691, M1-S690, M1-K689, M1-K688, M1-W687, M1-L686, M1-H685, M1-P684, M1-S683, M1-R682, M1-F681, M1-G680, M1-S679, M1-G678, M1-G677, M1-S676, M1-E675, M1-R674, M1-L673, M1-A672, M1-G671, M1-N670, M1-P669, M1-V668, M1-G667, M1-A666, M1-G665, M1-N664, M1-S663, M1-P662, M1-K661, M1-P660, M1-S659, M1-K658, M1-A657, M1-P656, M1-D655, M1-A654, M1-T653, M1-D652, M1-L651, M1-P650, M1-T649, M1-F648, M1-A647, M1-L646, M1-A645, M1-G644, M1-N643, M1-S642, M1-I641, M1-D640, M1-R639, M1-G638, M1-E637, M1-E636, M1-E635, M1-G634, M1-A633, M1-G632, M1-R631, M1-R630, M1-E629, M1-P628, M1-Q627, M1-G626, M1-D625, M1-M624, M1-E623, M1-R622, M1-F621, M1-S620, M1-S619, M1-S618, M1-R617, M1-K616, M1-P615, M1-P614, M1-T613, M1-P612, M1-A611, M1-T610, M1-K609, M1-K608, M1-K607, M1-K606, M1-K605, M1-I604, M1-L603, M1-A602, M1-S601, M1-F600, M1-L599, M1-N598, M1-T597, M1-K596, M1-K595, M1-D594, M1-K593, M1-P592, M1-L591, M1-L590, M1-R589, M1-E588, M1-D587, M1-E586, M1-N585, M1-L584, M1-G583, M1-G582, M1-E581, M1-P580, M1-P579, M1-G578, M1-R577, M1-E576, M1-K575, M1-R574, M1-P573, M1-L572, M1-L571, M1-P570, M1-S569, M1-V568, M1-A567, M1-P566, M1-E565, M1-H564, M1-D563, M1-L562, M1-P561, M1-D560, M1-S559, M1-E558, M1-G557, M1-Q556, M1-G555, M1-K554, M1-S553, M1-H552, M1-P551, M1-M550, M1-E549, M1-P548, M1-V547, M1-D546, M1-T545, M1-T544, M1-D543, M1-R542, M1-H541, M1-E540, M1-A539, M1-A538, M1-R537, M1-R536, M1-S535, M1-T534, M1-R533, M1-T532, M1-K531, M1-T530, M1-P529, M1-L528, M1-E527, M1-P526, M1-A525, M1-Q524, M1-L523, M1-L522, M1-T521, M1-T520, M1-V519, M1-A518, M1-G517, M1-R516, M1-V515, M1-G514, M1-Q513, M1-K512, M1-G511, M1-L510, M1-E509, M1-K508, M1-E507, M1-V506, M1-E505, M1-D504, M1-S503, M1-I502, M1-S501, M1-S500, M1-E499, M1-Q498, M1-F497, M1-M496, M1-T495, M1-E494, M1-F493, M1-A492, M1-Q491, M1-H490, M1-I489, M1-E488, M1-A487, M1-F486, M1-S485, M1-P484, M1-R483, M1-D482, M1-S481, M1-P480, M1-N479, M1-W478, M1-Q477, M1-W476, M1-C475, M1-A474, M1-R473, M1-M472, M1-L471, M1-E470, M1-Y469, M1-V468, M1-K467, M1-E466, M1-P465, M1-C464, M1-G463, M1-E462, M1-P461, M1-R460, M1-K459, M1-M458, M1-R457, M1-Y456, M1-D455, M1-K454, M1-E453, M1-L452, M1-L451, M1-E450, M1-Y449, M1-V448, M1-Q447, M1-S446, M1-R445, M1-D444, M1-I443, M1-G442, M1-P441, M1-Y440, M1-P439, M1-S438, M1-M437, M1-G436, M1-Y435, M1-T434, M1-A433, M1-I432, M1-E431, M1-W430, M1-L429, M1-L428, M1-V427, M1-G426, M1-F425, M1-A424, M1-W423, M1-V422, M1-D421, M1-S420, M1-K419, M1-I418, M1-S417, M1-F416, M1-K415, M1-N414, M1-Y413, M1-A412, M1-L411, M1-S410, M1-E409, M1-P408, M1-A407, M1-T406, M1-W405, M1-K404, M1-I403, M1-P402, M1-F401, M1-K400, M1-A399, M1-G398, M1-A397, M1-H396, M1-A395, M1-T394, M1-Y393, M1-T392, M1-D391, M1-G390, M1-T389, M1-M388, M1-L387, M1-R386, M1-S385, M1-L384, M1-G383, M1-F382, M1-D381, M1-A380, M1-V379, M1-K378, M1-V377, M1-L376, M1-H375, M1-N374, M1-E373, M1-G372, M1-V371, M1-L370, M1-C369, M1-N368, M1-R367, M1-A366, M1-A365, M1-L364, M1-D363, M1-R362, M1-H361, M1-I360, M1-F359, M1-N358, M1-K357, M1-K356, M1-E355, M1-L354, M1-Y353, M1-E352, M1-M351, M1-A350, M1-S349, M1-S348, M1-I347, M1-Q346, M1-T345, M1-A344, M1-M343, M1-Y342, M1-L341, M1-L340, M1-V339, M1-V338, M1-A337, M1-N336, M1-V335, M1-E334, M1-Q333, M1-R332, M1-N331, M1-C330, M1-E329, M1-R328, M1-L327, M1-Y326, M1-D325, M1-L324, M1-L323, M1-N322, M1-G321, M1-Y320, M1-T319, M1-M318, M1-F317, M1-E316, M1-T315, M1-I314, M1-I313, M1-Y312, M1-F311, M1-P310, M1-P309, M1-E308, M1-R307, M1-T306, M1-C305, M1-V304, M1-G303, M1-L302, M1-L301, M1-Q300, M1-V299, M1-L298, M1-N297, M1-P296, M1-H295, M1-K294, M1-I293, M1-E292, M1-K291, M1-M290, M1-V289, M1-A288, M1-A287, M1-E286, M1-K285, M1-L284, M1-F283, M1-E282, M1-E281, M1-V280, M1-E279, M1-M278, M1-T277, M1-D276, M1-E275, M1-K274, M1-L273, M1-T272, M1-K271, M1-V270, M1-A269, M1-V268, M1-T267, M1-L266, M1-S265, M1-Y264, M1-K263, M1-K262, M1-W261, M1-V260, M1-G259, M1-E258, M1-Y257, M1-V256, M1-E255, M1-G254, M1-Y253, M1-Q252, M1-G251, M1-G250, M1-G249, M1-L248, M1-K247, M1-H246, M1-K245, M1-M244, M1-T243, M1-I242, M1-D241, M1-T240, M1-R239, M1-E238, M1-M237, M1-E236, M1-W235, M1-K234, M1-D233, M1-Y232, M1-N231, M1-P230, M1-S229, M1-V228, M1-G227, M1-Y226, M1-V225, M1-T224, M1-P223, M1-K222, M1-N221, M1-R220, M1-K219, M1-P218, M1-A217, M1-P216, M1-Y215, M1-H214, M1-L213, M1-T212, M1-T211, M1-I210, M1-L209, M1-G208, M1-D207, M1-A206, M1-V205, M1-T204, M1-S203, M1-H202, M1-H201, M1-H200, M1-V199, M1-L198, M1-E197, M1-A196, M1-L195, M1-T194, M1-N193, M1-F192, M1-R191, M1-S190, M1-E189, M1-S188, M1-S187, M1-V186, M1-Y185, M1-L184, M1-K183, M1-G182, M1-D181, M1-S180, M1-A179, M1-T178, M1-N177, M1-I176, M1-R175, M1-Y174, M1-H173, M1-Y172, M1-V171, M1-R170, M1-G169, M1-E168, M1-Y167, M1-R166, M1-L165, M1-S164, M1-I163, M1-S162, M1-R161, M1-Q160, M1-S159, M1-P158, M1-S157, M1-S156, M1-E155, M1-S154, M1-E153, M1-R152, M1-V151, M1-L150, M1-F149, M1-S148, M1-G147, M1-N146, M1-I145, M1-G144, M1-S143, M1-S142, M1-L141, M1-P140, M1-Y139, M1-E138, M1-A137, M1-A136, M1-N135, M1-R134, M1-S133, M1-V132, M1-P131, M1-G130, M1-H129, M1-Y128, M1-W127, M1-S126, M1-H125, M1-K124, M1-E123, M1-L122, M1-S121, M1-N120, M1-V119, M1-P118, M1-T117, M1-I116, M1-Y115, M1-N114, M1-S113, M1-P112, M1-V111, M1-W110, M1-G109, M1-Q108, M1-G107, M1-N106, M1-K105, M1-T104, M1-Q103, M1-A102, M1-E110, M1-C100, M1-W99, M1-E98, M1-G97, M1-N96, M1-H95, M1-N94, M1-Y93, M1-G92, M1-L91, M1-V90, M1-R89, M1-L88, M1-K87, M1-E86, M1-G85, M1-K84, M1-T83, M1-I82, M1-S81, M1-L80, M1-T79, M1-N78, M1-D77, M1-G76, M1-S75, M1-A74, M1-V73, M1-F72, M1-D71, M1-Y70, M1-L69, M1-A68, M1-V67, M1-F66, M1-L65, M1-N64, M1-P63, M1-D62, M1-N61, M1-E60, M1-S59, M1-P58, M1-G57, M1-A56, M1-L55, M1-L54, M1-N53, M1-E52, M1-K51, M1-S50, M1-N49, M1-W48, M1-R47, M1-A46, M1-A45, M1-E44, M1-S43, M1-L42, M1-G41, M1-Q40, M1-P39, M1-E38, M1-F37, M1-D36, M1-S35, M1-A34, M1-V33, M1-P32, M1-R31, M1-Q30, M1-L29, M1-A28, M1-E27, M1-E26, M1-L25, M1-Y24, M1-C23, M1-S22, M1-S21, M1-S20, M1-S19, M1-S18, M1-L17, M1-G16, M1-K15, M1-K14, M1-S13, M1-K12, M1-C11, M1-G10, M1-V9, M1-L8, and/or M1-K7 of SEQ ID NO:2. Polynucleotide sequences encoding these polypeptides are also provided. The present invention also encompasses the use of these C-terminal BCR/ABL deletion polypeptides as immunogenic and/or antigenic epitopes as described elsewhere herein.

Representative PCR amplification conditions are provided below, although the skilled artisan would appreciate that other conditions may be required for efficient amplification. A 100 ul PCR reaction mixture may be prepared using 10 ng of the template DNA (cDNA clone of BCR/ABL), 200 uM 4dNTPs, 1 uM primers, 0.25 U Taq DNA polymerase (PE), and standard Taq DNA polymerase buffer. Typical PCR cycling condition are as follows:

20-25 cycles:
45 sec, 93 degrees
2 min, 50 degrees
2 min, 72 degrees

1 cycle: 10 min, 72 degrees

After the final extension step of PCR, 5 U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E. coli cells using methods provided herein and/or otherwise known in the art.

The 5′ primer sequence for amplifying any additional N-terminal deletion mutants may be determined by reference to the following formula: (S+(X*3)) to ((S+(X*3))+25), wherein ‘S’ is equal to the nucleotide position of the initiating start codon of the BCR/ABL gene (SEQ ID NO:1), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula: (S+(X*3)) to ((S+(X*3))-25), wherein ‘S’ is equal to the nucleotide position of the initiating start codon of the BCR/ABL gene (SEQ ID NO:1), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

Each recited range includes all combinations and sub-combinations of ranges, as well as specific numerals contained therein.

One skilled in the art could easily modify the exemplified studies to test the activity of polynucleotides of the invention (e.g., gene therapy), agonists, and/or antagonists of polynucleotides or polypeptides of the invention.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, Genbank Accession numbers, SWISS-PROT Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

1. A method for determining the responsiveness of an individual with a BCR-ABL associated disorder to treatment with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, comprising:
(a) screening a biological sample from said individual for the presence of at least one mutation in a BCR-ABL polypeptide sequence; wherein the at least one mutation is a E507G mutation; and wherein the presence of the at least one mutation is indicative of the individual being at least partially resistant to N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, therapy.

2. (canceled)

3. The method of claim 1 wherein the individual has not previously been treated with a kinase inhibitor.

4. The method of claim 1 wherein the individual has been previously treated with a kinase inhibitor and has developed at least partial resistance to the kinase inhibitor.

5. The method of claim 4 wherein the kinase inhibitor is N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof.

6. The method of claim 5 wherein the kinase inhibitor is imatinib, AMN107, PD180970, CGP76030, AP23464, SKI 606, or AZD0530.

7. The method of claim 1 wherein the BCR-ABL-associated disorder is leukemia, breast cancer, prostate cancer, lung cancer, colon cancer, melanoma, or solid tumors.

8. The method of claim 7 wherein the leukemia is chronic myeloid leukemia (CML), Ph+ALL, AML, imatinib-resistant CML, imatinib-intolerant CML, accelerated CML, or lymphoid blast phase CML.

9. The method of claim 1 wherein the sample is further screened for the presence of a E279K, F359C, F359I, L3641, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, T315A, E316G, F317L, F317I, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, F486S or any combination thereof, mutation.

10. A method of treating an individual suffering from a BCR-ABL-associated disorder comprising:
(a) determining whether a biological sample obtained from the individual comprises a BCR-ABL polypeptide having at least one mutation wherein the at least one mutation is a E507G mutation, wherein the presence of the at least one mutation is indicative of the patient being at least partially resistant to N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, therapy; and
(b) administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, to the individual.

11-19. (canceled)

20. A method of determining whether a test compound modulates the tyrosine kinase activity of a BCR-ABL polypeptide, wherein the BCR-ABL polypeptide comprises at least one of a E507G mutation, comprising:
(a) obtaining mammalian cells transfected with a construct encoding the BCR-ABL polypeptide so that the BCR-ABL polypeptide is expressed by the mammalian cells;
(b) contacting the mammalian cells with the test compound; and
(c) monitoring the mammalian cells for tyrosine kinase activity of the BCR-ABL polypeptide wherein a modulation in tyrosine kinase activity in the presence of the test compound identifies the test compound as a modulator of the BCR-ABL polypeptide.

21. The method of claim 20 wherein the test compound inhibits the tyrosine kinase activity of a BCR-ABL polypeptide.

22. A kit for use in determining treatment strategy for an individual with a BCR-ABL-associated disorder, comprising:
(a) a means for detecting a mutant BCR-ABL in a biological sample from said individual; and optionally
(b) instructions for use and interpretation of the kit results.

23. The kit of claim 22, wherein said kit comprises said instructions and wherein said treatment strategy comprises administration of a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate or solvate thereof.

24-28. (canceled)

29. A method for determining the responsiveness of an individual with a BCR-ABL associated disorder to treatment with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, comprising:
(a) screening a biological sample from said individual for the presence of at least one mutation in a BCR-ABL polypeptide sequence; wherein the presence of the at least one mutation is indicative of the individual being at least partially resistant to N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, therapy.

30. The method according to claim 29 wherein said at least one mutation resides at one or more of the following amino acid positions selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130 of SEQ ID NO:2.


SEQUENCE LISTING (16 sequences)

SEQ ID NO: 1 - DNA, 3393 bp, Homo sapiens
atgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatccgctgagcagcgggatcaatggcagcttcttggtgcgtgagagtgagagcagtcctagccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtag

SEQ ID NO: 2 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 3 - DNA, 22 bp, Artificial
  Synthesized oligonucleotide.
gaagcttctccctgacatccgt

SEQ ID NO: 4 - DNA, 25 bp, Artificial
  Synthesized oligonucleotide.
ccccacggacgccttgtttccccag

SEQ ID NO: 5 - DNA, 24 bp, Artificial
  Synthesized oligonucleotide.
gcgcaacaagcccactgtctatgg

SEQ ID NO: 6 - DNA, 24 bp, Bacteriophage M13
cgccagggttttcccagtcacgac

SEQ ID NO: 7 - DNA, 24 bp, Bacteriophage M13
agcggataacaatttcacacagga

SEQ ID NO: 8 - DNA, 20 bp, Artificial
  Synthesized oligonucleotide.
gcagagtcagaatccttcag

SEQ ID NO: 9 - DNA, 20 bp, Artificial
  Synthesized oligonucleotide.
tttgtaaaaggctgcccggc

SEQ ID NO: 10 - DNA, 30 bp, Artificial
  Synthesized oligonucleotide.
cggcattgcgggacacaggcccatggtacc

SEQ ID NO: 11 - DNA, 22 bp, Artificial
  Synthesized oligonucleotide.
tgaccaactcgtgtgtgaaact

SEQ ID NO: 12 - DNA, 34 bp, Artificial
  Synthesized oligonucleotide.
ggggaattcgccaccatgttggagatctgcctga

SEQ ID NO: 13 - DNA, 20 bp, Artificial
  Synthesized oligonucleotide.
gcagagtcagaatccttcag

SEQ ID NO: 14 - DNA, 20 bp, Artificial
  Synthesized oligonucleotide.
tttgtaaaaggctgcccggc

SEQ ID NO: 15 - DNA, 20 bp, Artificial
  Synthesized oligonucleotide.
ttcaccaccatggagaaggc

SEQ ID NO: 16 - DNA, 20 bp, Artificial
  Synthesized oligonucleotide.
caggaaatgagcttgacaaa